SEQUENCE LISTING (222 sequences)

SEQ ID NO: 1 - DNA, 60 bp, Artificial Sequence
  artificial promoter element
  modified_base (6)..(55): a, c, t, or g
  misc_feature (6)..(55): This region may encompass 0-50 nucleotides
tkacgnnnnnnnnnnnnnnnnnnnnnnnnnnnnnnnnnnnnnnnnnnnnnnnnnntkacg

SEQ ID NO: 2 - DNA, 62 bp, Artificial Sequence
  artificial promoter element
  modified_base (7)..(56): a, c, t, or g
  misc_feature (7)..(56): This region may encompass 0-50 nucleotides
cacgtgnnnnnnnnnnnnnnnnnnnnnnnnnnnnnnnnnnnnnnnnnnnnnnnnnncacgtg

SEQ ID NO: 3 - DNA, 10 bp, Artificial Sequence
  artificial promoter element
cyyyyyyyyc

SEQ ID NO: 4 - DNA, 10 bp, Artificial Sequence
  artificial promoter element
cctcctcctc

SEQ ID NO: 5 - DNA, 20 bp, Zea mays
ctgacgtaagcaatgacgaa

SEQ ID NO: 6 - DNA, 20 bp, Beta vulgaris
atgacgtaagtgaagacgca

SEQ ID NO: 7 - DNA, 20 bp, Beta vulgaris
atgacgaaagcactgacgaa

SEQ ID NO: 8 - DNA, 21 bp, Beta vulgaris
accacgtgcttggcacgtgat

SEQ ID NO: 9 - DNA, 20 bp, Artificial Sequence
  artificial promoter element
  misc_feature (12)..(12): n is a, c, g, or t
tgacgwaagsrntkacgmam

SEQ ID NO: 10 - DNA, 12 bp, Artificial Sequence
  artificial promoter element
  misc_feature (11)..(11): n is a, c, g, or t
vaaaatatctna

SEQ ID NO: 11 - DNA, 10 bp, Artificial Sequence
  artificial promoter element
  misc_feature (1)..(1): n is a, c, g, or t
  misc_feature (5)..(5): n is a, c, g, or t
  misc_feature (10)..(10): n is a, c, g, or t
nbvgnatctn

SEQ ID NO: 12 - DNA, 20 bp, Artificial Sequence
  artificial promoter element
  misc_feature (12)..(19): n is a, c, g, or t
scacacgtgyvnnnnnnnnb

SEQ ID NO: 13 - DNA, 18 bp, Artificial Sequence
  artificial promoter element
  misc_feature (4)..(4): n is a, c, g, or t
  misc_feature (17)..(17): n is a, c, g, or t
aaanvaaaaagtaaaanv

SEQ ID NO: 14 - DNA, 19 bp, Artificial Sequence
  artificial promoter element
gghcacgtggcvcvvvvgv

SEQ ID NO: 15 - DNA, 14 bp, Artificial Sequence
  artificial promoter element
  misc_feature (3)..(4): n is a, c, g, or t
dwnnggataagvwt

SEQ ID NO: 16 - DNA, 13 bp, Artificial Sequence
  artificial promoter element
  misc_feature (10)..(13): n is a, c, g, or t
ccaccgacannnn

SEQ ID NO: 17 - DNA, 13 bp, Artificial Sequence
  artificial promoter element
  misc_feature (1)..(1): n is a, c, g, or t
nggwcacgtggca

SEQ ID NO: 18 - DNA, 14 bp, Artificial Sequence
  artificial promoter element
cgtgagtcacgtga

SEQ ID NO: 19 - DNA, 11 bp, Artificial Sequence
  artificial promoter element
  misc_feature (1)..(1): n is a, c, g, or t
nvawaaagtaa

SEQ ID NO: 20 - DNA, 12 bp, Artificial Sequence
  artificial promoter element
bggggracabgt

SEQ ID NO: 21 - DNA, 21 bp, Artificial Sequence
  artificial promoter element
  misc_feature (16)..(16): n is a, c, g, or t
  misc_feature (20)..(20): n is a, c, g, or t
bbgcacavwtcccvangtvna

SEQ ID NO: 22 - DNA, 11 bp, Artificial Sequence
  artificial promoter element
  misc_feature (1)..(1): n is a, c, g, or t
nrcacgtgbsv

SEQ ID NO: 23 - DNA, 12 bp, Artificial Sequence
  artificial promoter element
  misc_feature (11)..(12): n is a, c, g, or t
vaaaatatctnn

SEQ ID NO: 24 - DNA, 15 bp, Artificial Sequence
  artificial promoter element
  misc_feature (2)..(2): n is a, c, g, or t
  misc_feature (5)..(5): n is a, c, g, or t
cnavngggagacavb

SEQ ID NO: 25 - DNA, 11 bp, Artificial Sequence
  artificial promoter element
  misc_feature (1)..(1): n is a, c, g, or t
ncaataattva

SEQ ID NO: 26 - DNA, 12 bp, Artificial Sequence
  artificial promoter element
  misc_feature (1)..(1): n is a, c, g, or t
  misc_feature (7)..(7): n is a, c, g, or t
ngtgggncccac

SEQ ID NO: 27 - DNA, 14 bp, Artificial Sequence
  artificial promoter element
  misc_feature (3)..(3): n is a, c, g, or t
wtntggataagvtt

SEQ ID NO: 28 - DNA, 24 bp, Artificial Sequence
  artificial promoter element
gagagagagagagagagagagaga

SEQ ID NO: 29 - DNA, 20 bp, Artificial Sequence
  artificial promoter element
  misc_feature (7)..(7): n is a, c, g, or t
  misc_feature (13)..(13): n is a, c, g, or t
aaaaagnaaaaanaaaaaaa

SEQ ID NO: 30 - DNA, 12 bp, Artificial Sequence
  artificial promoter element
gshsacgtggca

SEQ ID NO: 31 - DNA, 11 bp, Artificial Sequence
  artificial promoter element
  misc_feature (10)..(11): n is a, c, g, or t
agaatattcnn

SEQ ID NO: 32 - DNA, 15 bp, Artificial Sequence
  artificial promoter element
  misc_feature (3)..(3): n is a, c, g, or t
  misc_feature (6)..(6): n is a, c, g, or t
ccnccnccaccgcca

SEQ ID NO: 33 - DNA, 16 bp, Artificial Sequence
  artificial promoter element
  misc_feature (12)..(15): n is a, c, g, or t
gatgacgtcaynnnnb

SEQ ID NO: 34 - DNA, 11 bp, Artificial Sequence
  artificial promoter element
  misc_feature (1)..(1): n is a, c, g, or t
  misc_feature (11)..(11): n is a, c, g, or t
nbcaacttgcn

SEQ ID NO: 35 - DNA, 11 bp, Artificial Sequence
  artificial promoter element
  misc_feature (11)..(11): n is a, c, g, or t
ccaccgacavn

SEQ ID NO: 36 - DNA, 14 bp, Artificial Sequence
  artificial promoter element
  misc_feature (2)..(3): n is a, c, g, or t
  misc_feature (14)..(14): n is a, c, g, or t
vnngccgacaagvn

SEQ ID NO: 37 - DNA, 11 bp, Artificial Sequence
  artificial promoter element
  misc_feature (1)..(1): n is a, c, g, or t
  misc_feature (10)..(10): n is a, c, g, or t
naaaaccgsna

SEQ ID NO: 38 - DNA, 10 bp, Artificial Sequence
  artificial promoter element
  misc_feature (1)..(1): n is a, c, g, or t
  misc_feature (9)..(10): n is a, c, g, or t
nggtaaaann

SEQ ID NO: 39 - DNA, 21 bp, Artificial Sequence
  artificial promoter element
  misc_feature (6)..(6): n is a, c, g, or t
  misc_feature (8)..(9): n is a, c, g, or t
  misc_feature (21)..(21): n is a, c, g, or t
vcvbtnbnngtgggtcccacn

SEQ ID NO: 40 - DNA, 14 bp, Artificial Sequence
  artificial promoter element
  misc_feature (3)..(3): n is a, c, g, or t
ggnggaggcggtgg

SEQ ID NO: 41 - DNA, 11 bp, Artificial Sequence
  artificial promoter element
  misc_feature (1)..(1): n is a, c, g, or t
  misc_feature (11)..(11): n is a, c, g, or t
nbgccgacatn

SEQ ID NO: 42 - DNA, 10 bp, Artificial Sequence
  artificial promoter element
  misc_feature (1)..(1): n is a, c, g, or t
  misc_feature (10)..(10): n is a, c, g, or t
nggttaaaan

SEQ ID NO: 43 - DNA, 12 bp, Artificial Sequence
  artificial promoter element
tgctgacgtggc

SEQ ID NO: 44 - DNA, 11 bp, Artificial Sequence
  artificial promoter element
  misc_feature (1)..(1): n is a, c, g, or t
  misc_feature (11)..(11): n is a, c, g, or t
ntgtcggtgvn

SEQ ID NO: 45 - DNA, 11 bp, Artificial Sequence
  artificial promoter element
  misc_feature (1)..(1): n is a, c, g, or t
  misc_feature (11)..(11): n is a, c, g, or t
nbcaccgacan

SEQ ID NO: 46 - DNA, 12 bp, Artificial Sequence
  artificial promoter element
  misc_feature (10)..(12): n is a, c, g, or t
agatattttnnn

SEQ ID NO: 47 - DNA, 11 bp, Artificial Sequence
  artificial promoter element
  misc_feature (3)..(3): n is a, c, g, or t
ccnccaccgcc

SEQ ID NO: 48 - DNA, 19 bp, Artificial Sequence
  artificial promoter element
  misc_feature (2)..(3): n is a, c, g, or t
  misc_feature (6)..(6): n is a, c, g, or t
  misc_feature (9)..(9): n is a, c, g, or t
cnnccnbcnccgccgccgc

SEQ ID NO: 49 - DNA, 10 bp, Artificial Sequence
  artificial promoter element
  misc_feature (9)..(9): n is a, c, g, or t
sawaaagtna

SEQ ID NO: 50 - DNA, 19 bp, Artificial Sequence
  artificial promoter element
  misc_feature (3)..(3): n is a, c, g, or t
  misc_feature (6)..(8): n is a, c, g, or t
gvngvnnntggcggcggvg

SEQ ID NO: 51 - DNA, 19 bp, Artificial Sequence
  artificial promoter element
  misc_feature (6)..(7): n is a, c, g, or t
aaaaannaaaaagtvaaaa

SEQ ID NO: 52 - DNA, 11 bp, Artificial Sequence
  artificial promoter element
  misc_feature (1)..(1): n is a, c, g, or t
nvccgacaaav

SEQ ID NO: 53 - DNA, 12 bp, Artificial Sequence
  artificial promoter element
  misc_feature (11)..(12): n is a, c, g, or t
vvacgacaagnn

SEQ ID NO: 54 - DNA, 18 bp, Artificial Sequence
  artificial promoter element
ttaccaaaaatggaaaaw

SEQ ID NO: 55 - DNA, 12 bp, Artificial Sequence
  artificial promoter element
tgccacgtcabc

SEQ ID NO: 56 - DNA, 11 bp, Artificial Sequence
  artificial promoter element
  misc_feature (11)..(11): n is a, c, g, or t
tgcaagttgvn

SEQ ID NO: 57 - DNA, 15 bp, Artificial Sequence
  artificial promoter element
  misc_feature (2)..(3): n is a, c, g, or t
annaaaaaggbaaaa

SEQ ID NO: 58 - DNA, 15 bp, Artificial Sequence
  artificial promoter element
  misc_feature (2)..(2): n is a, c, g, or t
  misc_feature (11)..(14): n is a, c, g, or t
anggtaaaaannnna

SEQ ID NO: 59 - DNA, 12 bp, Artificial Sequence
  artificial promoter element
  misc_feature (1)..(1): n is a, c, g, or t
ntgacagctbgb

SEQ ID NO: 60 - DNA, 15 bp, Artificial Sequence
  artificial promoter element
  misc_feature (3)..(3): n is a, c, g, or t
ccnccgccgccgcca

SEQ ID NO: 61 - DNA, 10 bp, Artificial Sequence
  artificial promoter element
  misc_feature (10)..(10): n is a, c, g, or t
bcaatcattn

SEQ ID NO: 62 - DNA, 11 bp, Artificial Sequence
  artificial promoter element
  misc_feature (11)..(11): n is a, c, g, or t
btgtcggtggn

SEQ ID NO: 63 - DNA, 16 bp, Artificial Sequence
  artificial promoter element
  misc_feature (13)..(13): n is a, c, g, or t
bgtaatgattacncbc

SEQ ID NO: 64 - DNA, 11 bp, Artificial Sequence
  artificial promoter element
  misc_feature (1)..(1): n is a, c, g, or t
nccgccgccgb

SEQ ID NO: 65 - DNA, 11 bp, Artificial Sequence
  artificial promoter element
cgcacgtgtgv

SEQ ID NO: 66 - DNA, 11 bp, Artificial Sequence
  artificial promoter element
  misc_feature (1)..(2): n is a, c, g, or t
  misc_feature (11)..(11): n is a, c, g, or t
nncaatcattn

SEQ ID NO: 67 - DNA, 11 bp, Artificial Sequence
  artificial promoter element
  misc_feature (11)..(11): n is a, c, g, or t
ccaccgacavn

SEQ ID NO: 68 - DNA, 10 bp, Artificial Sequence
  artificial promoter element
  misc_feature (1)..(2): n is a, c, g, or t
nnaaaaagtg

SEQ ID NO: 69 - DNA, 10 bp, Artificial Sequence
  artificial promoter element
  misc_feature (3)..(3): n is a, c, g, or t
  misc_feature (7)..(8): n is a, c, g, or t
ccnaatnngg

SEQ ID NO: 70 - DNA, 21 bp, Artificial Sequence
  artificial promoter element
  misc_feature (6)..(7): n is a, c, g, or t
  misc_feature (18)..(20): n is a, c, g, or t
cgtgannvbcacgtgvbnnnw

SEQ ID NO: 71 - DNA, 11 bp, Artificial Sequence
  artificial promoter element
  misc_feature (1)..(1): n is a, c, g, or t
nbtgtcggtgg

SEQ ID NO: 72 - DNA, 12 bp, Artificial Sequence
  artificial promoter element
grtgacgtggcc

SEQ ID NO: 73 - DNA, 18 bp, Artificial Sequence
  artificial promoter element
  misc_feature (6)..(6): n is a, c, g, or t
  misc_feature (11)..(11): n is a, c, g, or t
  misc_feature (17)..(17): n is a, c, g, or t
tttccnttttnggaaant

SEQ ID NO: 74 - DNA, 10 bp, Artificial Sequence
  artificial promoter element
acacgtggca

SEQ ID NO: 75 - DNA, 10 bp, Artificial Sequence
  artificial promoter element
  misc_feature (1)..(2): n is a, c, g, or t
nnttaacaga

SEQ ID NO: 76 - DNA, 12 bp, Artificial Sequence
  artificial promoter element
  misc_feature (1)..(1): n is a, c, g, or t
  misc_feature (10)..(10): n is a, c, g, or t
ntgcggcggngv

SEQ ID NO: 77 - DNA, 13 bp, Artificial Sequence
  artificial promoter element
vcaaaaawrgaaa

SEQ ID NO: 78 - DNA, 10 bp, Artificial Sequence
  artificial promoter element
  misc_feature (1)..(1): n is a, c, g, or t
nawttaatgc

SEQ ID NO: 79 - DNA, 15 bp, Artificial Sequence
  artificial promoter element
  misc_feature (2)..(2): n is a, c, g, or t
tnccaaaaatggaaa

SEQ ID NO: 80 - DNA, 10 bp, Artificial Sequence
  artificial promoter element
  misc_feature (10)..(10): n is a, c, g, or t
tgtcggtggn

SEQ ID NO: 81 - DNA, 14 bp, Artificial Sequence
  artificial promoter element
  misc_feature (3)..(3): n is a, c, g, or t
  misc_feature (6)..(6): n is a, c, g, or t
ccnccnccaccgcc

SEQ ID NO: 82 - DNA, 11 bp, Artificial Sequence
  artificial promoter element
  misc_feature (11)..(11): n is a, c, g, or t
vcccgacaagn

SEQ ID NO: 83 - DNA, 14 bp, Artificial Sequence
  artificial promoter element
arcgggggacacgt

SEQ ID NO: 84 - DNA, 11 bp, Artificial Sequence
  artificial promoter element
  misc_feature (1)..(1): n is a, c, g, or t
  misc_feature (11)..(11): n is a, c, g, or t
ncagacagcvn

SEQ ID NO: 85 - DNA, 10 bp, Artificial Sequence
  artificial promoter element
  misc_feature (1)..(2): n is a, c, g, or t
  misc_feature (10)..(10): n is a, c, g, or t
nnacacgtgn

SEQ ID NO: 86 - DNA, 16 bp, Artificial Sequence
  artificial promoter element
  misc_feature (7)..(8): n is a, c, g, or t
cttgbvnnvcaagtha

SEQ ID NO: 87 - DNA, 18 bp, Artificial Sequence
  artificial promoter element
  misc_feature (2)..(2): n is a, c, g, or t
  misc_feature (5)..(5): n is a, c, g, or t
  misc_feature (8)..(8): n is a, c, g, or t
cnccnccnccgccgccgb

SEQ ID NO: 88 - DNA, 30 bp, Artificial Sequence
  artificial promoter element
agagagagagagagagagagagagagagag

SEQ ID NO: 89 - DNA, 10 bp, Artificial Sequence
  artificial promoter element
  misc_feature (10)..(10): n is a, c, g, or t
bcaatcattn

SEQ ID NO: 90 - DNA, 21 bp, Artificial Sequence
  artificial promoter element
ctctctctctctctctctctc

SEQ ID NO: 91 - DNA, 10 bp, Artificial Sequence
  artificial promoter element
  misc_feature (10)..(10): n is a, c, g, or t
caataattgn

SEQ ID NO: 92 - DNA, 10 bp, Artificial Sequence
  artificial promoter element
cgsygccccc

SEQ ID NO: 93 - DNA, 10 bp, Artificial Sequence
  artificial promoter element
ccawawatag

SEQ ID NO: 94 - DNA, 10 bp, Artificial Sequence
  artificial promoter element
atgtcggyvv

SEQ ID NO: 95 - DNA, 15 bp, Artificial Sequence
  artificial promoter element
  misc_feature (7)..(7): n is a, c, g, or t
cgtgbbnbgcacgtg

SEQ ID NO: 96 - DNA, 11 bp, Artificial Sequence
  artificial promoter element
vtgacgtggca

SEQ ID NO: 97 - DNA, 15 bp, Artificial Sequence
  artificial promoter element
  misc_feature (6)..(6): n is a, c, g, or t
  misc_feature (14)..(14): n is a, c, g, or t
tttccntttttggna

SEQ ID NO: 98 - DNA, 11 bp, Artificial Sequence
  artificial promoter element
bbrccgacatv

SEQ ID NO: 99 - DNA, 17 bp, Artificial Sequence
  artificial promoter element
  misc_feature (4)..(4): n is a, c, g, or t
  misc_feature (15)..(15): n is a, c, g, or t
vbtnccawatatagnav

SEQ ID NO: 100 - DNA, 25 bp, Artificial Sequence
  artificial promoter element
  misc_feature (6)..(6): n is a, c, g, or t
  misc_feature (13)..(13): n is a, c, g, or t
  misc_feature (20)..(21): n is a, c, g, or t
  misc_feature (23)..(23): n is a, c, g, or t
tttttnbtttttnctttttnntntt

SEQ ID NO: 101 - DNA, 14 bp, Artificial Sequence
  artificial promoter element
  misc_feature (12)..(12): n is a, c, g, or t
awccttatcttnwt

SEQ ID NO: 102 - DNA, 10 bp, Artificial Sequence
  artificial promoter element
  misc_feature (2)..(2): n is a, c, g, or t
cnccaccgac

SEQ ID NO: 103 - DNA, 15 bp, Artificial Sequence
  artificial promoter element
  misc_feature (13)..(13): n is a, c, g, or t
ttttaaatttttnaa

SEQ ID NO: 104 - DNA, 14 bp, Artificial Sequence
  artificial promoter element
  misc_feature (11)..(11): n is a, c, g, or t
vcgggggacangtc

SEQ ID NO: 105 - DNA, 15 bp, Artificial Sequence
  artificial promoter element
  misc_feature (6)..(6): n is a, c, g, or t
  misc_feature (11)..(11): n is a, c, g, or t
tttccnttttnggaa

SEQ ID NO: 106 - DNA, 17 bp, Artificial Sequence
  artificial promoter element
  misc_feature (2)..(3): n is a, c, g, or t
  misc_feature (5)..(8): n is a, c, g, or t
vnngnnnntgtcggtgg

SEQ ID NO: 107 - DNA, 15 bp, Artificial Sequence
  artificial promoter element
  misc_feature (3)..(3): n is a, c, g, or t
  misc_feature (15)..(15): n is a, c, g, or t
ggnggtggcggtggn

SEQ ID NO: 108 - DNA, 21 bp, Artificial Sequence
  artificial promoter element
  misc_feature (3)..(3): n is a, c, g, or t
  misc_feature (5)..(5): n is a, c, g, or t
  misc_feature (7)..(7): n is a, c, g, or t
  misc_feature (16)..(16): n is a, c, g, or t
vananwnwggataagnatvvd

SEQ ID NO: 109 - DNA, 12 bp, Artificial Sequence
  artificial promoter element
  misc_feature (1)..(1): n is a, c, g, or t
nbatgtcggyvv

SEQ ID NO: 110 - DNA, 14 bp, Artificial Sequence
  artificial promoter element
  misc_feature (10)..(10): n is a, c, g, or t
  misc_feature (12)..(12): n is a, c, g, or t
awccttatcntnat

SEQ ID NO: 111 - DNA, 15 bp, Artificial Sequence
  artificial promoter element
vavvgggggacavbb

SEQ ID NO: 112 - DNA, 11 bp, Artificial Sequence
  artificial promoter element
  misc_feature (2)..(3): n is a, c, g, or t
wnnggataagg

SEQ ID NO: 113 - DNA, 11 bp, Artificial Sequence
  artificial promoter element
cgvwthccgag

SEQ ID NO: 114 - DNA, 10 bp, Artificial Sequence
  artificial promoter element
  misc_feature (1)..(1): n is a, c, g, or t
ngacacgtgb

SEQ ID NO: 115 - DNA, 10 bp, Artificial Sequence
  artificial promoter element
caccgacatw

SEQ ID NO: 116 - DNA, 13 bp, Artificial Sequence
  artificial promoter element
bgbbcaattattg

SEQ ID NO: 117 - DNA, 11 bp, Artificial Sequence
  artificial promoter element
bcacacgtgcg

SEQ ID NO: 118 - DNA, 29 bp, Artificial Sequence
  artificial promoter element
ctaaaccctaaaccctaaaccctaaaccc

SEQ ID NO: 119 - DNA, 16 bp, Artificial Sequence
  artificial promoter element
  misc_feature (7)..(9): n is a, c, g, or t
cacgtgnnncacgtgb

SEQ ID NO: 120 - DNA, 11 bp, Artificial Sequence
  artificial promoter element
  misc_feature (2)..(2): n is a, c, g, or t
  misc_feature (10)..(10): n is a, c, g, or t
wntggataanv

SEQ ID NO: 121 - DNA, 11 bp, Artificial Sequence
  artificial promoter element
  misc_feature (2)..(2): n is a, c, g, or t
  misc_feature (11)..(11): n is a, c, g, or t
vnagatacgvn

SEQ ID NO: 122 - DNA, 19 bp, Artificial Sequence
  artificial promoter element
  misc_feature (3)..(3): n is a, c, g, or t
  misc_feature (11)..(12): n is a, c, g, or t
  misc_feature (18)..(18): n is a, c, g, or t
btnactttttnnbttttnb

SEQ ID NO: 123 - DNA, 14 bp, Artificial Sequence
  artificial promoter element
  misc_feature (11)..(14): n is a, c, g, or t
ccaccgacavnnnn

SEQ ID NO: 124 - DNA, 16 bp, Artificial Sequence
  artificial promoter element
  misc_feature (2)..(2): n is a, c, g, or t
  misc_feature (9)..(9): n is a, c, g, or t
  misc_feature (12)..(13): n is a, c, g, or t
  misc_feature (15)..(15): n is a, c, g, or t
gncggtggngvnnvng

SEQ ID NO: 125 - DNA, 14 bp, Artificial Sequence
  artificial promoter element
  misc_feature (3)..(3): n is a, c, g, or t
  misc_feature (13)..(13): n is a, c, g, or t
vvnvcacgtgdvna

SEQ ID NO: 126 - DNA, 12 bp, Artificial Sequence
  artificial promoter element
tgccacgtcabc

SEQ ID NO: 127 - DNA, 10 bp, Artificial Sequence
  artificial promoter element
bcaccgacah

SEQ ID NO: 128 - DNA, 21 bp, Artificial Sequence
  artificial promoter element
  misc_feature (2)..(2): n is a, c, g, or t
  misc_feature (4)..(5): n is a, c, g, or t
  misc_feature (15)..(18): n is a, c, g, or t
  misc_feature (20)..(20): n is a, c, g, or t
vnbnnvvcacgtgbnnnnsnb

SEQ ID NO: 129 - DNA, 12 bp, Artificial Sequence
  artificial promoter element
  misc_feature (12)..(12): n is a, c, g, or t
ccgccgccaccn

SEQ ID NO: 130 - DNA, 11 bp, Artificial Sequence
  artificial promoter element
  misc_feature (1)..(1): n is a, c, g, or t
  misc_feature (9)..(9): n is a, c, g, or t
naaaaagtnaa

SEQ ID NO: 131 - DNA, 15 bp, Artificial Sequence
  artificial promoter element
  misc_feature (2)..(5): n is a, c, g, or t
vnnnngbcacgtgrb

SEQ ID NO: 132 - DNA, 13 bp, Artificial Sequence
  artificial promoter element
tgctgacgtggca

SEQ ID NO: 133 - DNA, 16 bp, Artificial Sequence
  artificial promoter element
  misc_feature (3)..(3): n is a, c, g, or t
  misc_feature (6)..(6): n is a, c, g, or t
  misc_feature (10)..(11): n is a, c, g, or t
  misc_feature (14)..(14): n is a, c, g, or t
ttnccnaatnnggnaa

SEQ ID NO: 134 - DNA, 15 bp, Artificial Sequence
  artificial promoter element
tbccaaaaatggvaa

SEQ ID NO: 135 - DNA, 15 bp, Artificial Sequence
  artificial promoter element
  misc_feature (3)..(3): n is a, c, g, or t
  misc_feature (6)..(6): n is a, c, g, or t
ccnbcnccaccghca

SEQ ID NO: 136 - DNA, 13 bp, Artificial Sequence
  artificial promoter element
  misc_feature (4)..(4): n is a, c, g, or t
  misc_feature (11)..(12): n is a, c, g, or t
tggncggtggnnv

SEQ ID NO: 137 - DNA, 10 bp, Artificial Sequence
  artificial promoter element
  misc_feature (9)..(10): n is a, c, g, or t
bccacgtcnn

SEQ ID NO: 138 - DNA, 15 bp, Artificial Sequence
  artificial promoter element
  misc_feature (12)..(12): n is a, c, g, or t
  misc_feature (15)..(15): n is a, c, g, or t
aaccttatccanwwn

SEQ ID NO: 139 - DNA, 18 bp, Artificial Sequence
  artificial promoter element
ttwccaaaaawggaaaaa

SEQ ID NO: 140 - DNA, 16 bp, Artificial Sequence
  artificial promoter element
cgggggacatgtcggg

SEQ ID NO: 141 - DNA, 16 bp, Artificial Sequence
  artificial promoter element
tttttgtcgttttstb

SEQ ID NO: 142 - DNA, 10 bp, Artificial Sequence
  artificial promoter element
bkgggaccac

SEQ ID NO: 143 - DNA, 17 bp, Artificial Sequence
  artificial promoter element
  misc_feature (11)..(11): n is a, c, g, or t
  misc_feature (14)..(14): n is a, c, g, or t
  misc_feature (17)..(17): n is a, c, g, or t
vcggcggcggnggnggn

SEQ ID NO: 144 - DNA, 12 bp, Artificial Sequence
  artificial promoter element
  misc_feature (2)..(3): n is a, c, g, or t
  misc_feature (5)..(5): n is a, c, g, or t
bnnbntcactct

SEQ ID NO: 145 - DNA, 11 bp, Artificial Sequence
  artificial promoter element
vgctcagcgcs

SEQ ID NO: 146 - DNA, 12 bp, Artificial Sequence
  artificial promoter element
gccacgtcagca

SEQ ID NO: 147 - DNA, 20 bp, Artificial Sequence
  artificial promoter element
  misc_feature (12)..(12): n is a, c, g, or t
tttacttgttbnvcaagtaa

SEQ ID NO: 148 - DNA, 10 bp, Artificial Sequence
  artificial promoter element
  misc_feature (9)..(10): n is a, c, g, or t
haataattnn

SEQ ID NO: 149 - DNA, 14 bp, Artificial Sequence
  artificial promoter element
  misc_feature (2)..(3): n is a, c, g, or t
anngtaatgattab

SEQ ID NO: 150 - DNA, 10 bp, Artificial Sequence
  artificial promoter element
aaavcgcgtg

SEQ ID NO: 151 - DNA, 11 bp, Artificial Sequence
  artificial promoter element
bbrccgacatv

SEQ ID NO: 152 - DNA, 18 bp, Artificial Sequence
  artificial promoter element
  misc_feature (13)..(13): n is a, c, g, or t
actttccwttttnggaaa

SEQ ID NO: 153 - DNA, 12 bp, Artificial Sequence
  artificial promoter element
atcvaagattcy

SEQ ID NO: 154 - DNA, 16 bp, Artificial Sequence
  artificial promoter element
  misc_feature (3)..(3): n is a, c, g, or t
  misc_feature (6)..(7): n is a, c, g, or t
  misc_feature (10)..(11): n is a, c, g, or t
  misc_feature (14)..(14): n is a, c, g, or t
ttnccnnatnnggnaa

SEQ ID NO: 155 - DNA, 20 bp, Artificial Sequence
  artificial promoter element
  misc_feature (2)..(2): n is a, c, g, or t
  misc_feature (8)..(8): n is a, c, g, or t
  misc_feature (11)..(11): n is a, c, g, or t
cnccdccnccnccrccgccv

SEQ ID NO: 156 - DNA, 15 bp, Artificial Sequence
  artificial promoter element
  misc_feature (5)..(7): n is a, c, g, or t
wttannnvttaatta

SEQ ID NO: 157 - DNA, 12 bp, Artificial Sequence
  artificial promoter element
  misc_feature (2)..(2): n is a, c, g, or t
  misc_feature (12)..(12): n is a, c, g, or t
cnccaccgacan

SEQ ID NO: 158 - DNA, 18 bp, Artificial Sequence
  artificial promoter element
  misc_feature (2)..(2): n is a, c, g, or t
  misc_feature (13)..(14): n is a, c, g, or t
  misc_feature (16)..(17): n is a, c, g, or t
bnatgtcggtgvnnvnnv

SEQ ID NO: 159 - DNA, 14 bp, Artificial Sequence
  artificial promoter element
  misc_feature (12)..(13): n is a, c, g, or t
ggcggcggcggnng

SEQ ID NO: 160 - DNA, 10 bp, Artificial Sequence
  artificial promoter element
  misc_feature (10)..(10): n is a, c, g, or t
scgdatcttn

SEQ ID NO: 161 - DNA, 15 bp, Artificial Sequence
  artificial promoter element
  misc_feature (1)..(3): n is a, c, g, or t
  misc_feature (12)..(15): n is a, c, g, or t
nnntgccgacannnn

SEQ ID NO: 162 - DNA, 15 bp, Artificial Sequence
  artificial promoter element
  misc_feature (2)..(4): n is a, c, g, or t
  misc_feature (7)..(7): n is a, c, g, or t
  misc_feature (13)..(13): n is a, c, g, or t
tnnnaanattctntt

SEQ ID NO: 163 - DNA, 14 bp, Artificial Sequence
  artificial promoter element
ctttctattttbgg

SEQ ID NO: 164 - DNA, 15 bp, Artificial Sequence
  artificial promoter element
  misc_feature (14)..(14): n is a, c, g, or t
vvvwttagggtttnb

SEQ ID NO: 165 - DNA, 13 bp, Artificial Sequence
  artificial promoter element
  misc_feature (12)..(13): n is a, c, g, or t
vcaagacaagann

SEQ ID NO: 166 - DNA, 13 bp, Artificial Sequence
  artificial promoter element
  misc_feature (12)..(13): n is a, c, g, or t
tcaatgattgann

SEQ ID NO: 167 - DNA, 13 bp, Artificial Sequence
  artificial promoter element
  misc_feature (2)..(2): n is a, c, g, or t
  misc_feature (13)..(13): n is a, c, g, or t
wntggataaggtn

SEQ ID NO: 168 - DNA, 12 bp, Artificial Sequence
  artificial promoter element
  misc_feature (12)..(12): n is a, c, g, or t
accaataattgn

SEQ ID NO: 169 - DNA, 17 bp, Artificial Sequence
  artificial promoter element
  misc_feature (2)..(2): n is a, c, g, or t
  misc_feature (4)..(7): n is a, c, g, or t
bnbnnnnbbyrccgaca

SEQ ID NO: 170 - DNA, 13 bp, Artificial Sequence
  artificial promoter element
  misc_feature (8)..(8): n is a, c, g, or t
aaaaagtnvcttt

SEQ ID NO: 171 - DNA, 10 bp, Artificial Sequence
  artificial promoter element
ggcacgtgcc

SEQ ID NO: 172 - DNA, 11 bp, Artificial Sequence
  artificial promoter element
aaccttatchw

SEQ ID NO: 173 - DNA, 10 bp, Artificial Sequence
  artificial promoter element
yaatsattgb

SEQ ID NO: 174 - DNA, 13 bp, Artificial Sequence
  artificial promoter element
  misc_feature (2)..(3): n is a, c, g, or t
wnnggataagvtt

SEQ ID NO: 175 - DNA, 21 bp, Artificial Sequence
  artificial promoter element
  misc_feature (3)..(3): n is a, c, g, or t
  misc_feature (7)..(7): n is a, c, g, or t
  misc_feature (13)..(13): n is a, c, g, or t
ttntttntttttnvctttttb

SEQ ID NO: 176 - DNA, 11 bp, Artificial Sequence
  artificial promoter element
  misc_feature (10)..(11): n is a, c, g, or t
bcrccgacann

SEQ ID NO: 177 - DNA, 13 bp, Artificial Sequence
  artificial promoter element
  misc_feature (11)..(11): n is a, c, g, or t
tbgtcggtggngv

SEQ ID NO: 178 - DNA, 14 bp, Artificial Sequence
  artificial promoter element
  misc_feature (13)..(14): n is a, c, g, or t
gccacgtcagcann

SEQ ID NO: 179 - DNA, 25 bp, Artificial Sequence
  artificial promoter element
tttyactttttbttttttttttttt

SEQ ID NO: 180 - DNA, 21 bp, Artificial Sequence
  artificial promoter element
  misc_feature (3)..(5): n is a, c, g, or t
  misc_feature (14)..(14): n is a, c, g, or t
  misc_feature (16)..(16): n is a, c, g, or t
  misc_feature (20)..(20): n is a, c, g, or t
aannntaattaatnantwant

SEQ ID NO: 181 - DNA, 11 bp, Artificial Sequence
  artificial promoter element
  misc_feature (11)..(11): n is a, c, g, or t
gcattaattan

SEQ ID NO: 182 - DNA, 11 bp, Artificial Sequence
  artificial promoter element
ygcacgtgtgv

SEQ ID NO: 183 - DNA, 10 bp, Artificial Sequence
  artificial promoter element
dtgtcggtgv

SEQ ID NO: 184 - DNA, 986 bp, Zea mays
ggttggcatacagatcacctctaaaagtgttttctatgctagtggttcaaataaaatttcaaagaccaaggaaagggggtcattatatgtttataatacaacctgccagtaatagctatcactgtcatcaggttgtattaccaaccgacaatgataactatatatgtattattggatcgaacatgttatattatcaattgacaacgacaactatctattaatgggttatattgccaactgacagtgataactatcttattattgcatgttggatataaaatccgataacgataaagccgttgtttatgaaccgatagttatatttttatcattcttgatatctaaaattgtctgggagtgatgggctgatatttattgtctatgttgtagtagtgtatatttgtgttggtgactaagcaatgcttatatataaactgttgaccagagaaggatctattatatactccctcagtctcaaaatatatagtttttttgtagtctacttgttttctgtccacattcatttaaatgaaaatgaatatagacgtgcgtgcaaactacttaatgaatgtatgattaatctaaaacgaattatatttttagaacggagtgagtactttgcatcttctcccatcactctcctgcatattcatcctaatatatcccacgtggatggagcgcatgcatgcatatctcacattcatagtttttgacttggttaagatcccatcctatgggctatggctttgtttgcaccggccggaccaattctctttctcttatccggtttgttcaatggcgattggtttacaccgttgtgtggactatgtcgcagactcgcagtgaggtttaatttgtttgcatgcatgcccaactaataaactgtaaaacccgcgtccggcgtccctacaaataacaccgatcacttgttctcgtactagtgagatagggcactcttgcttggcgtagcattagggacgacgacg

SEQ ID NO: 185 - DNA, 986 bp, Artificial Sequence
  artificial promoter sequence
ggttggcatacagatcacctctaaaagtgttttctatgctagtggttcaaataaaatttcaaagaccaaggaaagggggtcattatatgtttataatacaacctgccagtaatagctatcactgtcatcaggttgtattaccaaccgacaatgataactatatatgtattattggatcgaacatgttatattatcaattgacaacgacaactatctattaatgggttatattgccaactgacagtgataactatcttattattgcatgttggatataaaatccgataacgataaagccgttgtttatgaaccgatagttatatttttatcattcttgatatctaaaattgtctgggagtgatgggctgatatttattgtctatgttgtagtagtgtatatttgtgttggtgactaagcaatgcttatatataaactgttgaccagagaaggatctattatatactccctcagtctcaaaatatatagtttttttgtagtctacttgttttctgtccacattcatttaaatgaaaatgaatatagacgtgcgtgcaaactacttaatgaatgtatgattaatctaaaacgaattatatttttagaacggagtgagtactttgcatcttctcccatcactctcctgcatattcatcctaatatatcccacgtggatggagcgcatgcatgcatatctcacattcatagtttttgacttggttaagatcccatcctatgggctatggctttgtttgcaccggccggaccaattctctttctcttatccggtttgttcaatggcgattggtttacaccgttgtgtggactatgtcgcagactcgcagtgagctataaatagtttgcatgcatgcccaactaataaactgtaaaacccgcgtccggcgtccctacaaataacaccgatcacttgttctcgtactagtgagatagggcactcttgcttggcgtagcattagggacgacgacg

SEQ ID NO: 186 - DNA, 986 bp, Artificial Sequence
  artificial promoter sequence
ggttggcatacagatcacctctaaaagtgttttctatgctagtggttcaaataaaatttcaaagaccaaggaaagggggtcattatatgtttataatacaacctgccagtaatagctatcactgtcatcaggttgtattaccaaccgacaatgataactatatatgtattattggatcgaacatgttatattatcaattgacaacgacaactatctattaatgggttatattgccaactgacagtgataactatcttattattgcatgttggatataaaatccgataacgataaagccgttgtttatgaaccgatagttatatttttatcattcttgatatctaaaattgtctgggagtgatgggctgatatttattgtctatgttgtagtagtgtatatttgtgttggtgactaagcaatgcttatatataaactgttgaccagagaaggatctattatatactccctcagtctcaaaatatatagtttttttgtagtctacttgttttctgtccacattcatttaaatgaaaatgaatatagacgtgcgtgcaaactacttaatgaatgtatgattaatctaaaacgaattatatttttagaacggagtgagtactttgcatcttctcccatcactctcctgcatattcatcctaatatatcccacgtggatggagcgcatgcatgcatatctcacattcatagtttttgacttggttaagatcccatcctatgggctatggctttgtttgcaccggccggaccaattctctttctcttatccggtttgttcaatggcgattggtttacaccgttgtgtggactatgtcgcagactcgcagtgaggtttaatttgtttgcatgcatgcccaactaataaactgtaaaacccgcgtccggcgtccctataaataacaccgatcacttgttctcgtactagtgagatagggcactcttgcttggcgtagcattagggacgacgacg

SEQ ID NO: 187 - DNA, 1007 bp, Artificial Sequence
  artificial promoter sequence
ggttggcatacagatcacctctaaaagtgttttctatgctagtggttcaaataaaatttcaaagaccaaggaaagggggtcattatatgtttataatacaacctgccagtaatagctatcactgtcatcaggttgtattaccaaccgacaatgataactatatatgtattattggatcgaacatgttatattatcaattgacaacgacaactatctattaatgggttatattgccaactgacagtgataactatcttattattgcatgttggatataaaatccgataacgataaagccgttgtttatgaaccgatagttatatttttatcattcttgatatctaaaattgtctgggagtgatgggctgatatttattgtctatgttgtagtagtgtatatttgtgttggtgactaagcaatgcttatatataaactgttgaccagagaaggatctattatatactccctcagtctcaaaatatatagtttttttgtagtctacttgttttctgtccacattcatttaaatgaaaatgaatatagacgtgcgtgcaaactacttaatgaatgtatgattaatctaaaacgaattatatttttagaacggagtgagtactttgcatcttctcccatcactctcctgcatattcatcctaatatatcccacgtggatggagcgcatgcatgcatatctcacattcatagtttttgacttggttaagatcccatcctatgggctatggctttgtttgcaccggccggaccaattctctttctcttatccggtttgttcaatggcgattggtttacaccgttgtgtggactatgtcgcagactcgcagtgaggtttaatttgtttgcatgcatagctgacgtaagcaatgacgaacccaactaataaactgtaaaacccgcgtccggcgtccctacaaataacaccgatcacttgttctcgtactagtgagatagggcactcttgcttggcgtagcattagggacgacgacg

SEQ ID NO: 188 - DNA, 1007 bp, Artificial Sequence
  artificial promoter sequence
ggttggcatacagatcacctctaaaagtgttttctatgctagtggttcaaataaaatttcaaagaccaaggaaagggggtcattatatgtttataatacaacctgccagtaatagctatcactgtcatcaggttgtattaccaaccgacaatgataactatatatgtattattggatcgaacatgttatattatcaattgacaacgacaactatctattaatgggttatattgccaactgacagtgataactatcttattattgcatgttggatataaaatccgataacgataaagccgttgtttatgaaccgatagttatatttttatcattcttgatatctaaaattgtctgggagtgatgggctgatatttattgtctatgttgtagtagtgtatatttgtgttggtgactaagcaatgcttatatataaactgttgaccagagaaggatctattatatactccctcagtctcaaaatatatagtttttttgtagtctacttgttttctgtccacattcatttaaatgaaaatgaatatagacgtgcgtgcaaactacttaatgaatgtatgattaatctaaaacgaattatatttttagaacggagtgagtactttgcatcttctcccatcactctcctgcatattcatcctaatatatcccacgtggatggagcgcatgcatgcatatctcacattcatagtttttgacttggttaagatcccatcctatgggctatggctttgtttgcaccggccggaccaattctctttctcttatccggtttgttcaatggcgattggtttacaccgttgtgtggactatgtcgcagactcgcagtgagctataaatagtttgcatgcatagctgacgtaagcaatgacgaacccaactaataaactgtaaaacccgcgtccggcgtccctacaaataacaccgatcacttgttctcgtactagtgagatagggcactcttgcttggcgtagcattagggacgacgacg

SEQ ID NO: 189 - DNA, 1007 bp, Artificial Sequence
  artificial promoter sequence
ggttggcatacagatcacctctaaaagtgttttctatgctagtggttcaaataaaatttcaaagaccaaggaaagggggtcattatatgtttataatacaacctgccagtaatagctatcactgtcatcaggttgtattaccaaccgacaatgataactatatatgtattattggatcgaacatgttatattatcaattgacaacgacaactatctattaatgggttatattgccaactgacagtgataactatcttattattgcatgttggatataaaatccgataacgataaagccgttgtttatgaaccgatagttatatttttatcattcttgatatctaaaattgtctgggagtgatgggctgatatttattgtctatgttgtagtagtgtatatttgtgttggtgactaagcaatgcttatatataaactgttgaccagagaaggatctattatatactccctcagtctcaaaatatatagtttttttgtagtctacttgttttctgtccacattcatttaaatgaaaatgaatatagacgtgcgtgcaaactacttaatgaatgtatgattaatctaaaacgaattatatttttagaacggagtgagtactttgcatcttctcccatcactctcctgcatattcatcctaatatatcccacgtggatggagcgcatgcatgcatatctcacattcatagtttttgacttggttaagatcccatcctatgggctatggctttgtttgcaccggccggaccaattctctttctcttatccggtttgttcaatggcgattggtttacaccgttgtgtggactatgtcgcagactcgcagtgaggtttaatttgtttgcatgcatagctgacgtaagcaatgacgaacccaactaataaactgtaaaacccgcgtccggcgtccctataaataacaccgatcacttgttctcgtactagtgagatagggcactcttgcttggcgtagcattagggacgacgacg

SEQ ID NO: 190 - DNA, 1330 bp, Beta vulgaris
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagttaacgttgaaaattgaaatccatacatttgtagactttgtagtcatacttgtcaatcttaccccttcacagattgatcttccacctcatacttctcacttctcctccaaaaaaaacattaaattacttcctcactcaaaactcaacccctctcctcattccctccctcaaatttgacaatttctatgtgctccacccttcttcagtaatctttcttcaccacgccatttccattttctctctcctccaaatcc

SEQ ID NO: 191 - DNA, 1330 bp, Artificial Sequence
  artificial promoter sequence
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagttaacgttgaaaattgaaatccatacatttgtagactttgtagtcatacttgtcaatcttacccctataaatattgatcttccacctcatacttctcacttctcctccaaaaaaaacattaaattacttcctcactcaaaactcaacccctctcctcattccctccctcaaatttgacaatttctatgtgctccacccttcttcagtaatctttcttcaccacgccatttccattttctctctcctccaaatcc

SEQ ID NO: 192 - DNA, 1330 bp, Artificial Sequence
  artificial promoter sequence
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagttaacgttgaaaattgaaatccatacatttgtagactttgtagtcatacttgtcaatcttaccccttcacagattgatcttccacctcatacttctcacttctcctataaataaaacattaaattacttcctcactcaaaactcaacccctctcctcattccctccctcaaatttgacaatttctatgtgctccacccttcttcagtaatctttcttcaccacgccatttccattttctctctcctccaaatcc

SEQ ID NO: 193 - DNA, 1330 bp, Artificial Sequence
  artificial promoter sequence
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagttaacgttgaaaattgaaatccatacatttgtagactttgtagtcatacttgtcaatcttaccccttcacagattgatcttccacctcatacttctcacttctcctccaaaaaaaacattaaattacttcctcactcaaaactcaacccctataaatattccctccctcaaatttgacaatttctatgtgctccacccttcttcagtaatctttcttcaccacgccatttccattttctctctcctccaaatcc

SEQ ID NO: 194 - DNA, 1352 bp, Artificial Sequence
  artificial promoter sequence
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagatgacgtaagtgaagacgcaccttaacgttgaaaattgaaatccatacatttgtagactttgtagtcatacttgtcaatcttaccccttcacagattgatcttccacctcatacttctcacttctcctccaaaaaaaacattaaattacttcctcactcaaaactcaacccctctcctcattccctccctcaaatttgacaatttctatgtgctccacccttcttcagtaatctttcttcaccacgccatttccattttctctctcctccaaatcc

SEQ ID NO: 195 - DNA, 1352 bp, Artificial Sequence
  artificial promoter sequence
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagatgacgtaagtgaagacgcaccttaacgttgaaaattgaaatccatacatttgtagactttgtagtcatacttgtcaatcttacccctataaatattgatcttccacctcatacttctcacttctcctccaaaaaaaacattaaattacttcctcactcaaaactcaacccctctcctcattccctccctcaaatttgacaatttctatgtgctccacccttcttcagtaatctttcttcaccacgccatttccattttctctctcctccaaatcc

SEQ ID NO: 196 - DNA, 1352 bp, Artificial Sequence
  artificial promoter sequence
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagatgacgtaagtgaagacgcaccttaacgttgaaaattgaaatccatacatttgtagactttgtagtcatacttgtcaatcttaccccttcacagattgatcttccacctcatacttctcacttctcctataaataaaacattaaattacttcctcactcaaaactcaacccctctcctcattccctccctcaaatttgacaatttctatgtgctccacccttcttcagtaatctttcttcaccacgccatttccattttctctctcctccaaatcc

SEQ ID NO: 197 - DNA, 1352 bp, Artificial Sequence
  artificial promoter sequence
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagatgacgtaagtgaagacgcaccttaacgttgaaaattgaaatccatacatttgtagactttgtagtcatacttgtcaatcttaccccttcacagattgatcttccacctcatacttctcacttctcctccaaaaaaaacattaaattacttcctcactcaaaactcaacccctataaatattccctccctcaaatttgacaatttctatgtgctccacccttcttcagtaatctttcttcaccacgccatttccattttctctctcctccaaatcc

SEQ ID NO: 198 - DNA, 20 bp, Zea mays
ctgacgcaagcaatgacgaa

SEQ ID NO: 199 - DNA, 21 bp, Beta vulgaris
tacacgtgtaaatcacgtgat

SEQ ID NO: 200 - DNA, 32 bp, Zea mays
aggggcataatggtaaaggggcataatggtaa

SEQ ID NO: 201 - DNA, 19 bp, Zea mays
ctggacacaggtcacacag

SEQ ID NO: 202 - DNA, 22 bp, Zea mays
acgtcattttcattagtatgat

SEQ ID NO: 203 - DNA, 15 bp, Artificial Sequence
  Y-patch E085
tctctctctctctct

SEQ ID NO: 204 - DNA, 30 bp, Artificial Sequence
  Y-patch E086
tctctctctctctctctctctctctctctc

SEQ ID NO: 205 - DNA, 1353 bp, Artificial Sequence
  BvHPPD1-50-E133
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagtacacgtgtaaatcacgtgatccttaacgttgaaaattgaaatccatacatttgtagactttgtagtcatacttgtcaatcttaccccttcacagattgatcttccacctcatacttctcacttctcctccaaaaaaaacattaaattacttcctcactcaaaactcaacccctctcctcattccctccctcaaatttgacaatttctatgtgctccacccttcttcagtaatctttcttcaccacgccatttccattttctctctcctccaaatcc

SEQ ID NO: 206 - DNA, 1353 bp, Artificial Sequence
  BvHPPD1v5-50-E133
ggggtcaaattcaaggcaggccaacctatgtttatctaacttgtattagaccaaactcgtttcttgaatattctgaatttgattaagttggcatttgaattaaagactcactcaatatgttgaattattgcaatgattaaatttaaaatcaagtcattttgggttggtttcgggtcaacctgaaatcgaaaagatcgggttcaaattataattttcgacctgaaaggctgaaatcagaatcaagtcaagctttgatcagaggtaagtgacccgaagtagaaatcaggtgaacttgaaattaatccaactcgaacttattgctacccctacattattaatatagacaaacgacatgtaaagataattattgtatactacaataatatctaataataatgtactctactggagtaataattaagcaatataaaaagttgtgaaaattatcaatctcgcaactccaaaataaattgtctacattataaatcaaaatctacgactctacgggtaagttacataaatgataatgtaaaatggtaagttatatcaatattaacattgtttataattagttagcaactgtgcccgaattatacacgaactaatatgattgtaaagtacttccatttgcttagtgaaacttaccaaatttataaaattgtaattgatccattttacacgtggcactaaaaaaatgagaataagtgtaatgatgacatgtggcgaaatgagtaatagcttgacacataggcaaatttttctagctttatatatatatatatatatagatgattattatgacctgacccatccttaaaaaaatcgagttagaccgacccttgacatgccccaaccggccaacccactaacaattacaatcaacccgtagtaccaacccaacccaaatccaaccctgcccatcagacacgctactcacaagtcaccaacaaagaagcaaaaagaccaaaccaaagtcacataaatcccaaagttaaagctgtaacgttcacgtaattcaattctccaactttatcttcttcatctttctatgttcaatagtcaataccacggtaaagtacacgtgtaaatcacgtgatccttaacgttgaaaattgaaatccatacatttgtagactttgtagtcatacttgtcaatcttaccccttcacagattgatcttccacctcatacttctcacttctcctccaaaaaaaacattaaattacttcctcactcaaaactcaacccctataaatattccctccctcaaatttgacaatttctatgtgctccacccttcttcagtaatctttcttcaccacgccatttccattttctctctcctccaaatcc

SEQ ID NO: 207 - DNA, 1409 bp, Beta vulgaris
cgcaacataagcccggtttagttcacctcaaattttttgatctaaacttatctgatctgatttgatttgaacttattttttctgatctggtctgatttgattacagatcttatcttttagatttttttcaatatataagaaagaacatagtcatgtggagtcttgtttgatttgtctcaatgagtactttattaatattcaattattataatttttactaatatgtaatcaaagatatttaatagtataatgatttttaaatataagcaagatctgatatgatcttattttttctgatctgatctgaacttattttttctgatctgatctgattagatctgaaataagcaaaaataaggtgaactcaacctactacctagccataatttctctcttctgttttacgtgaaatataggatgaaatgatactatttattgattaattttgatttcaatttatcaccattatataaagaaaaatatagtcaagtgggatattgctaaattcgtcttgatgcatatatattatcaaattataaattttttaatattttttacgcgtgcacaattaaagatattaatggttaaataagcgcgttggattgcgtgaaaaaacaaatgtttcatttaaaatagaacggaggaattatactattttaggtagttaaacattagttgtcttttctaaatttaactataactctcttcaagtatttttttttatgaaaatgagcgaccaaacgctatgatctagacatgattagattccagatctcgcgtaccactcccgcaagactttaccaactaagctagatgataaatctattagaattcccatttattatccacgggtacttttatccagccctatcagccttccttaaaaactagtgcttgtttggttgacattgtttatattaatatgggaagttagaattcttaggaattgaaatttttgatattgtttggttgacaaattcataggaaattccacttcctacaaaatgaaggaaactaggtaagtgaaaattcccatgaaaattttcattttccattaaaacaccaaacaacatttagcaaatcctaaaaagtacaattacatgagaatataaacttccaaaaaattcatacttccttccttcaaccaaacaaacactttttttttttttttttgaggggaaccaaacaaacacttaaaaataagccgaacccatatgttacaattaataaaaaactcatattccaaacctcaatcaatcattctcaatcccctcctccatccctagggtataataattgtatataatgactgtaatccaccatgtccataccatccattatttccaatcatcatcttctctctacaaatcacaaaaccttctttagtttgcactctccaagtctccacc

SEQ ID NO: 208 - DNA, 1409 bp, Artificial Sequence
  BvHPPD2v5
cgcaacataagcccggtttagttcacctcaaattttttgatctaaacttatctgatctgatttgatttgaacttattttttctgatctggtctgatttgattacagatcttatcttttagatttttttcaatatataagaaagaacatagtcatgtggagtcttgtttgatttgtctcaatgagtactttattaatattcaattattataatttttactaatatgtaatcaaagatatttaatagtataatgatttttaaatataagcaagatctgatatgatcttattttttctgatctgatctgaacttattttttctgatctgatctgattagatctgaaataagcaaaaataaggtgaactcaacctactacctagccataatttctctcttctgttttacgtgaaatataggatgaaatgatactatttattgattaattttgatttcaatttatcaccattatataaagaaaaatatagtcaagtgggatattgctaaattcgtcttgatgcatatatattatcaaattataaattttttaatattttttacgcgtgcacaattaaagatattaatggttaaataagcgcgttggattgcgtgaaaaaacaaatgtttcatttaaaatagaacggaggaattatactattttaggtagttaaacattagttgtcttttctaaatttaactataactctcttcaagtatttttttttatgaaaatgagcgaccaaacgctatgatctagacatgattagattccagatctcgcgtaccactcccgcaagactttaccaactaagctagatgataaatctattagaattcccatttattatccacgggtacttttatccagccctatcagccttccttaaaaactagtgcttgtttggttgacattgtttatattaatatgggaagttagaattcttaggaattgaaatttttgatattgtttggttgacaaattcataggaaattccacttcctacaaaatgaaggaaactaggtaagtgaaaattcccatgaaaattttcattttccattaaaacaccaaacaacatttagcaaatcctaaaaagtacaattacatgagaatataaacttccaaaaaattcatacttccttccttcaaccaaacaaacactttttttttttttttttgaggggaaccaaacaaacacttaaaaataagccgaacccatatgttacaattaataaaaaactcatattccaaacctcaatcaatcattctcaatcccctcctccatccctagggtataataattgtatactataaatataatccaccatgtccataccatccattatttccaatcatcatcttctctctacaaatcacaaaaccttctttagtttgcactctccaagtctccacc

SEQ ID NO: 209 - DNA, 1431 bp, Artificial Sequence
  BvHPPD2-50-E038h
cgcaacataagcccggtttagttcacctcaaattttttgatctaaacttatctgatctgatttgatttgaacttattttttctgatctggtctgatttgattacagatcttatcttttagatttttttcaatatataagaaagaacatagtcatgtggagtcttgtttgatttgtctcaatgagtactttattaatattcaattattataatttttactaatatgtaatcaaagatatttaatagtataatgatttttaaatataagcaagatctgatatgatcttattttttctgatctgatctgaacttattttttctgatctgatctgattagatctgaaataagcaaaaataaggtgaactcaacctactacctagccataatttctctcttctgttttacgtgaaatataggatgaaatgatactatttattgattaattttgatttcaatttatcaccattatataaagaaaaatatagtcaagtgggatattgctaaattcgtcttgatgcatatatattatcaaattataaattttttaatattttttacgcgtgcacaattaaagatattaatggttaaataagcgcgttggattgcgtgaaaaaacaaatgtttcatttaaaatagaacggaggaattatactattttaggtagttaaacattagttgtcttttctaaatttaactataactctcttcaagtatttttttttatgaaaatgagcgaccaaacgctatgatctagacatgattagattccagatctcgcgtaccactcccgcaagactttaccaactaagctagatgataaatctattagaattcccatttattatccacgggtacttttatccagccctatcagccttccttaaaaactagtgcttgtttggttgacattgtttatattaatatgggaagttagaattcttaggaattgaaatttttgatattgtttggttgacaaattcataggaaattccacttcctacaaaatgaaggaaactaggtaagtgaaaattcccatgaaaattttcattttccattaaaacaccaaacaacatttagcaaatcctaaaaagtacaattacatgagaatataaacttccaaaaaattcatacttccttccttcaaccaaacaaacactttttttttttttttttgaggggaaccaaacaaacacttaaaaataagccgaacccatatgttacaattaataaaaaactcatattccaaacctcaatcaatcattctcaatcccctcctagatgacgaaagcactgacgaaccatccctagggtataataattgtatataatgactgtaatccaccatgtccataccatccattatttccaatcatcatcttctctctacaaatcacaaaaccttctttagtttgcactctccaagtctccacc

SEQ ID NO: 210 - DNA, 1432 bp, Artificial Sequence
  BvHPPD2-50-E128
cgcaacataagcccggtttagttcacctcaaattttttgatctaaacttatctgatctgatttgatttgaacttattttttctgatctggtctgatttgattacagatcttatcttttagatttttttcaatatataagaaagaacatagtcatgtggagtcttgtttgatttgtctcaatgagtactttattaatattcaattattataatttttactaatatgtaatcaaagatatttaatagtataatgatttttaaatataagcaagatctgatatgatcttattttttctgatctgatctgaacttattttttctgatctgatctgattagatctgaaataagcaaaaataaggtgaactcaacctactacctagccataatttctctcttctgttttacgtgaaatataggatgaaatgatactatttattgattaattttgatttcaatttatcaccattatataaagaaaaatatagtcaagtgggatattgctaaattcgtcttgatgcatatatattatcaaattataaattttttaatattttttacgcgtgcacaattaaagatattaatggttaaataagcgcgttggattgcgtgaaaaaacaaatgtttcatttaaaatagaacggaggaattatactattttaggtagttaaacattagttgtcttttctaaatttaactataactctcttcaagtatttttttttatgaaaatgagcgaccaaacgctatgatctagacatgattagattccagatctcgcgtaccactcccgcaagactttaccaactaagctagatgataaatctattagaattcccatttattatccacgggtacttttatccagccctatcagccttccttaaaaactagtgcttgtttggttgacattgtttatattaatatgggaagttagaattcttaggaattgaaatttttgatattgtttggttgacaaattcataggaaattccacttcctacaaaatgaaggaaactaggtaagtgaaaattcccatgaaaattttcattttccattaaaacaccaaacaacatttagcaaatcctaaaaagtacaattacatgagaatataaacttccaaaaaattcatacttccttccttcaaccaaacaaacactttttttttttttttttgaggggaaccaaacaaacacttaaaaataagccgaacccatatgttacaattaataaaaaactcatattccaaacctcaatcaatcattctcaatcccctcctagaccacgtgcttggcacgtgatccatccctagggtataataattgtatataatgactgtaatccaccatgtccataccatccattatttccaatcatcatcttctctctacaaatcacaaaaccttctttagtttgcactctccaagtctccacc

SEQ ID NO: 211 - DNA, 1431 bp, Artificial Sequence
  BvHPPD2v5-50-E038h
cgcaacataagcccggtttagttcacctcaaattttttgatctaaacttatctgatctgatttgatttgaacttattttttctgatctggtctgatttgattacagatcttatcttttagatttttttcaatatataagaaagaacatagtcatgtggagtcttgtttgatttgtctcaatgagtactttattaatattcaattattataatttttactaatatgtaatcaaagatatttaatagtataatgatttttaaatataagcaagatctgatatgatcttattttttctgatctgatctgaacttattttttctgatctgatctgattagatctgaaataagcaaaaataaggtgaactcaacctactacctagccataatttctctcttctgttttacgtgaaatataggatgaaatgatactatttattgattaattttgatttcaatttatcaccattatataaagaaaaatatagtcaagtgggatattgctaaattcgtcttgatgcatatatattatcaaattataaattttttaatattttttacgcgtgcacaattaaagatattaatggttaaataagcgcgttggattgcgtgaaaaaacaaatgtttcatttaaaatagaacggaggaattatactattttaggtagttaaacattagttgtcttttctaaatttaactataactctcttcaagtatttttttttatgaaaatgagcgaccaaacgctatgatctagacatgattagattccagatctcgcgtaccactcccgcaagactttaccaactaagctagatgataaatctattagaattcccatttattatccacgggtacttttatccagccctatcagccttccttaaaaactagtgcttgtttggttgacattgtttatattaatatgggaagttagaattcttaggaattgaaatttttgatattgtttggttgacaaattcataggaaattccacttcctacaaaatgaaggaaactaggtaagtgaaaattcccatgaaaattttcattttccattaaaacaccaaacaacatttagcaaatcctaaaaagtacaattacatgagaatataaacttccaaaaaattcatacttccttccttcaaccaaacaaacactttttttttttttttttgaggggaaccaaacaaacacttaaaaataagccgaacccatatgttacaattaataaaaaactcatattccaaacctcaatcaatcattctcaatcccctcctagatgacgaaagcactgacgaaccatccctagggtataataattgtatactataaatataatccaccatgtccataccatccattatttccaatcatcatcttctctctacaaatcacaaaaccttctttagtttgcactctccaagtctccacc

SEQ ID NO: 212 - DNA, 1432 bp, Artificial Sequence
  BvHPPD2v5-50-E128
cgcaacataagcccggtttagttcacctcaaattttttgatctaaacttatctgatctgatttgatttgaacttattttttctgatctggtctgatttgattacagatcttatcttttagatttttttcaatatataagaaagaacatagtcatgtggagtcttgtttgatttgtctcaatgagtactttattaatattcaattattataatttttactaatatgtaatcaaagatatttaatagtataatgatttttaaatataagcaagatctgatatgatcttattttttctgatctgatctgaacttattttttctgatctgatctgattagatctgaaataagcaaaaataaggtgaactcaacctactacctagccataatttctctcttctgttttacgtgaaatataggatgaaatgatactatttattgattaattttgatttcaatttatcaccattatataaagaaaaatatagtcaagtgggatattgctaaattcgtcttgatgcatatatattatcaaattataaattttttaatattttttacgcgtgcacaattaaagatattaatggttaaataagcgcgttggattgcgtgaaaaaacaaatgtttcatttaaaatagaacggaggaattatactattttaggtagttaaacattagttgtcttttctaaatttaactataactctcttcaagtatttttttttatgaaaatgagcgaccaaacgctatgatctagacatgattagattccagatctcgcgtaccactcccgcaagactttaccaactaagctagatgataaatctattagaattcccatttattatccacgggtacttttatccagccctatcagccttccttaaaaactagtgcttgtttggttgacattgtttatattaatatgggaagttagaattcttaggaattgaaatttttgatattgtttggttgacaaattcataggaaattccacttcctacaaaatgaaggaaactaggtaagtgaaaattcccatgaaaattttcattttccattaaaacaccaaacaacatttagcaaatcctaaaaagtacaattacatgagaatataaacttccaaaaaattcatacttccttccttcaaccaaacaaacactttttttttttttttttgaggggaaccaaacaaacacttaaaaataagccgaacccatatgttacaattaataaaaaactcatattccaaacctcaatcaatcattctcaatcccctcctagaccacgtgcttggcacgtgatccatccctagggtataataattgtatactataaatataatccaccatgtccataccatccattatttccaatcatcatcttctctctacaaatcacaaaaccttctttagtttgcactctccaagtctccacc

SEQ ID NO: 213 - DNA, 991 bp, Beta vulgaris
aaagaaggaaaggaaggaatttgaacatgtgacctatcgttcacagcacctcaatcttaatcactagaccaaaacatccttggttcttgcgcaagaaggttggctagaaattttttgtaaaaacactagccccgctcagttcataatgagaatgtcgatgtcaccaaagggatattaaatgaatggaattgggatatggatggaatataatgaaatagagccactttgaggttccctatgaaatgaggcatggaagggagccactacgaaaaagttccgggagttacgaaggaagcttcgagctcatattggtcatgaacccgattactgagtctaataagttcaattgaaaagaaaaagtcttatgttctaaaagaacttttcgtgcggtttgcatgagttcatagtccatataatataatgcaggaatgaagttctcagttgattcttccacacccgtccctcaccccctaggccccaccttcaccccgccgaaaaaaataaagaaaatccaacgttatttttcttagaaatgacagtttgatatagaaaggaaaaataataataaaaaaaaaaagtgttggcgttttcattttcaacctcagtatgttggtttgccccaacaagttctgaaccaattggcgatgtaatcttataagaagaatctaacgttggtccattttgcttctacagttttgaaagttaggtgggccccattattatgttgatcctagaataattaattttggtaggctgagaagaggaaaaataaagaacaatgctaaaaacaagtgaaaaatatagttgcaactcatgatgcaacatgagatgcgatgaaatatgatagtaacttgagctcacaactctgtatataagtgctcatttggacacttattttctacaatttcctagtaactcagcttagcttcattcccgacttttttataaaagtcaggacgatcaatatctatctatttatctgtctgtctgt

SEQ ID NO: 214 - DNA, 1013 bp, Artificial Sequence
  BvFT2-50-E038h
aaagaaggaaaggaaggaatttgaacatgtgacctatcgttcacagcacctcaatcttaatcactagaccaaaacatccttggttcttgcgcaagaaggttggctagaaattttttgtaaaaacactagccccgctcagttcataatgagaatgtcgatgtcaccaaagggatattaaatgaatggaattgggatatggatggaatataatgaaatagagccactttgaggttccctatgaaatgaggcatggaagggagccactacgaaaaagttccgggagttacgaaggaagcttcgagctcatattggtcatgaacccgattactgagtctaataagttcaattgaaaagaaaaagtcttatgttctaaaagaacttttcgtgcggtttgcatgagttcatagtccatataatataatgcaggaatgaagttctcagttgattcttccacacccgtccctcaccccctaggccccaccttcaccccgccgaaaaaaataaagaaaatccaacgttatttttcttagaaatgacagtttgatatagaaaggaaaaataataataaaaaaaaaaagtgttggcgttttcattttcaacctcagtatgttggtttgccccaacaagttctgaaccaattggcgatgtaatcttataagaagaatctaacgttggtccattttgcttctacagttttgaaagttaggtgggccccattattatgttgatcctagaataattaattttggtaggctgagaagaggaaaaataaagaacaatgctaaaaacaagtgaaaaatatagttgcaactcatgatgcaacatgagatgcgatgaaatatgatagatgacgaaagcactgacgaacctaacttgagctcacaactctgtatataagtgctcatttggacacttattttctacaatttcctagtaactcagcttagcttcattcccgacttttttataaaagtcaggacgatcaatatctatctatttatctgtctgtctgt

SEQ ID NO: 215 - DNA, 1006 bp, Artificial Sequence
  BvFT2+40 -E085
aaagaaggaaaggaaggaatttgaacatgtgacctatcgttcacagcacctcaatcttaatcactagaccaaaacatccttggttcttgcgcaagaaggttggctagaaattttttgtaaaaacactagccccgctcagttcataatgagaatgtcgatgtcaccaaagggatattaaatgaatggaattgggatatggatggaatataatgaaatagagccactttgaggttccctatgaaatgaggcatggaagggagccactacgaaaaagttccgggagttacgaaggaagcttcgagctcatattggtcatgaacccgattactgagtctaataagttcaattgaaaagaaaaagtcttatgttctaaaagaacttttcgtgcggtttgcatgagttcatagtccatataatataatgcaggaatgaagttctcagttgattcttccacacccgtccctcaccccctaggccccaccttcaccccgccgaaaaaaataaagaaaatccaacgttatttttcttagaaatgacagtttgatatagaaaggaaaaataataataaaaaaaaaaagtgttggcgttttcattttcaacctcagtatgttggtttgccccaacaagttctgaaccaattggcgatgtaatcttataagaagaatctaacgttggtccattttgcttctacagttttgaaagttaggtgggccccattattatgttgatcctagaataattaattttggtaggctgagaagaggaaaaataaagaacaatgctaaaaacaagtgaaaaatatagttgcaactcatgatgcaacatgagatgcgatgaaatatgatagtaacttgagctcacaactctgtatataagtgctcatttggacacttattttctacaatttcctagtaactcagcttagcttcattcccgacttttttataaaagtctctctctctctctccaggacgatcaatatctatctatttatctgtctgtctgt

SEQ ID NO: 216 - DNA, 1021 bp, Artificial Sequence
  BvFT2+40 -E086
aaagaaggaaaggaaggaatttgaacatgtgacctatcgttcacagcacctcaatcttaatcactagaccaaaacatccttggttcttgcgcaagaaggttggctagaaattttttgtaaaaacactagccccgctcagttcataatgagaatgtcgatgtcaccaaagggatattaaatgaatggaattgggatatggatggaatataatgaaatagagccactttgaggttccctatgaaatgaggcatggaagggagccactacgaaaaagttccgggagttacgaaggaagcttcgagctcatattggtcatgaacccgattactgagtctaataagttcaattgaaaagaaaaagtcttatgttctaaaagaacttttcgtgcggtttgcatgagttcatagtccatataatataatgcaggaatgaagttctcagttgattcttccacacccgtccctcaccccctaggccccaccttcaccccgccgaaaaaaataaagaaaatccaacgttatttttcttagaaatgacagtttgatatagaaaggaaaaataataataaaaaaaaaaagtgttggcgttttcattttcaacctcagtatgttggtttgccccaacaagttctgaaccaattggcgatgtaatcttataagaagaatctaacgttggtccattttgcttctacagttttgaaagttaggtgggccccattattatgttgatcctagaataattaattttggtaggctgagaagaggaaaaataaagaacaatgctaaaaacaagtgaaaaatatagttgcaactcatgatgcaacatgagatgcgatgaaatatgatagtaacttgagctcacaactctgtatataagtgctcatttggacacttattttctacaatttcctagtaactcagcttagcttcattcccgacttttttataaaagtctctctctctctctctctctctctctctcccaggacgatcaatatctatctatttatctgtctgtctgt

SEQ ID NO: 217 - DNA, 1028 bp, Artificial Sequence
  BvFT2-50-E038h+40 -E085
aaagaaggaaaggaaggaatttgaacatgtgacctatcgttcacagcacctcaatcttaatcactagaccaaaacatccttggttcttgcgcaagaaggttggctagaaattttttgtaaaaacactagccccgctcagttcataatgagaatgtcgatgtcaccaaagggatattaaatgaatggaattgggatatggatggaatataatgaaatagagccactttgaggttccctatgaaatgaggcatggaagggagccactacgaaaaagttccgggagttacgaaggaagcttcgagctcatattggtcatgaacccgattactgagtctaataagttcaattgaaaagaaaaagtcttatgttctaaaagaacttttcgtgcggtttgcatgagttcatagtccatataatataatgcaggaatgaagttctcagttgattcttccacacccgtccctcaccccctaggccccaccttcaccccgccgaaaaaaataaagaaaatccaacgttatttttcttagaaatgacagtttgatatagaaaggaaaaataataataaaaaaaaaaagtgttggcgttttcattttcaacctcagtatgttggtttgccccaacaagttctgaaccaattggcgatgtaatcttataagaagaatctaacgttggtccattttgcttctacagttttgaaagttaggtgggccccattattatgttgatcctagaataattaattttggtaggctgagaagaggaaaaataaagaacaatgctaaaaacaagtgaaaaatatagttgcaactcatgatgcaacatgagatgcgatgaaatatgatagatgacgaaagcactgacgaacctaacttgagctcacaactctgtatataagtgctcatttggacacttattttctacaatttcctagtaactcagcttagcttcattcccgacttttttataaaagtctctctctctctctccaggacgatcaatatctatctatttatctgtctgtctgt

SEQ ID NO: 218 - DNA, 1043 bp, Artificial Sequence
  BvFT2-50-E038h+40 -E086
aaagaaggaaaggaaggaatttgaacatgtgacctatcgttcacagcacctcaatcttaatcactagaccaaaacatccttggttcttgcgcaagaaggttggctagaaattttttgtaaaaacactagccccgctcagttcataatgagaatgtcgatgtcaccaaagggatattaaatgaatggaattgggatatggatggaatataatgaaatagagccactttgaggttccctatgaaatgaggcatggaagggagccactacgaaaaagttccgggagttacgaaggaagcttcgagctcatattggtcatgaacccgattactgagtctaataagttcaattgaaaagaaaaagtcttatgttctaaaagaacttttcgtgcggtttgcatgagttcatagtccatataatataatgcaggaatgaagttctcagttgattcttccacacccgtccctcaccccctaggccccaccttcaccccgccgaaaaaaataaagaaaatccaacgttatttttcttagaaatgacagtttgatatagaaaggaaaaataataataaaaaaaaaaagtgttggcgttttcattttcaacctcagtatgttggtttgccccaacaagttctgaaccaattggcgatgtaatcttataagaagaatctaacgttggtccattttgcttctacagttttgaaagttaggtgggccccattattatgttgatcctagaataattaattttggtaggctgagaagaggaaaaataaagaacaatgctaaaaacaagtgaaaaatatagttgcaactcatgatgcaacatgagatgcgatgaaatatgatagatgacgaaagcactgacgaacctaacttgagctcacaactctgtatataagtgctcatttggacacttattttctacaatttcctagtaactcagcttagcttcattcccgacttttttataaaagtctctctctctctctctctctctctctctcccaggacgatcaatatctatctatttatctgtctgtctgt

SEQ ID NO: 219 - DNA, 986 bp, Artificial Sequence
  ZmCWI3v3-2-51-E039g
ggttggcatacagatcacctctaaaagtgttttctatgctagtggttcaaataaaatttcaaagaccaaggaaagggggtcattatatgtttataatacaacctgccagtaatagctatcactgtcatcaggttgtattaccaaccgacaatgataactatatatgtattattggatcgaacatgttatattatcaattgacaacgacaactatctattaatgggttatattgccaactgacagtgataactatcttattattgcatgttggatataaaatccgataacgataaagccgttgtttatgaaccgatagttatatttttatcattcttgatatctaaaattgtctgggagtgatgggctgatatttattgtctatgttgtagtagtgtatatttgtgttggtgactaagcaatgcttatatataaactgttgaccagagaaggatctattatatactccctcagtctcaaaatatatagtttttttgtagtctacttgttttctgtccacattcatttaaatgaaaatgaatatagacgtgcgtgcaaactacttaatgaatgtatgattaatctaaaacgaattatatttttagaacggagtgagtactttgcatcttctcccatcactctcctgcatattcatcctaatatatcccacgtggatggagcgcatgcatgcatatctcacattcatagtttttgacttggttaagatcccatcctatgggctatggctttgtttgcaccggccggaccaattctctttctcttatccggtttgttcaatggcgattggtttacaccgttgtgtggactatgtcgcagactcgcagtgaggtttaatttgtttgcatgcatctgacgtaagcaatgacgaaaacccgcgtccggcgtccctataaataacaccgatcacttgttctcgtactagtgagatagggcactcttgcttggcgtagcattagggacgacgacg

SEQ ID NO: 220 - DNA, 986 bp, Artificial Sequence
  ZmCWI3v3-2-59-E039g
ggttggcatacagatcacctctaaaagtgttttctatgctagtggttcaaataaaatttcaaagaccaaggaaagggggtcattatatgtttataatacaacctgccagtaatagctatcactgtcatcaggttgtattaccaaccgacaatgataactatatatgtattattggatcgaacatgttatattatcaattgacaacgacaactatctattaatgggttatattgccaactgacagtgataactatcttattattgcatgttggatataaaatccgataacgataaagccgttgtttatgaaccgatagttatatttttatcattcttgatatctaaaattgtctgggagtgatgggctgatatttattgtctatgttgtagtagtgtatatttgtgttggtgactaagcaatgcttatatataaactgttgaccagagaaggatctattatatactccctcagtctcaaaatatatagtttttttgtagtctacttgttttctgtccacattcatttaaatgaaaatgaatatagacgtgcgtgcaaactacttaatgaatgtatgattaatctaaaacgaattatatttttagaacggagtgagtactttgcatcttctcccatcactctcctgcatattcatcctaatatatcccacgtggatggagcgcatgcatgcatatctcacattcatagtttttgacttggttaagatcccatcctatgggctatggctttgtttgcaccggccggaccaattctctttctcttatccggtttgttcaatggcgattggtttacaccgttgtgtggactatgtcgcagactcgcagtgaggtttaatttgtttctgacgtaagcaatgacgaaaactgtaaaacccgcgtccggcgtccctataaataacaccgatcacttgttctcgtactagtgagatagggcactcttgcttggcgtagcattagggacgacgacg

SEQ ID NO: 221 - DNA, 14 bp, Artificial Sequence
  Description of Artificial Sequence Synthetic oligonucleotide
  misc_feature (8)..(12): n is a, c, g, or t
arggryannnnngt

SEQ ID NO: 222 - DNA, 14 bp, Artificial Sequence
  Description of Artificial Sequence Synthetic oligonucleotide
aggggcataatggt

(19) United States
(12) Patent Application Publication
(10) Pub. No.: US 2025/0243498 A1
(43) Pub. Date: Jul. 31, 2025

(54) SYNERGISTIC PROMOTER ACTIVATION BY COMBINING CPE AND CRE MODIFICATIONS

(71) Applicant: KWS SAAT SE & Co. KGaA, Einbeck (DE)

(72) Inventors: Fridtjof WELTMEIER, Einbeck (DE); Corinna STREITNER, Melle (DE)

(73) Assignee: KWS SAAT SE & Co. KGaA, Einbeck (DE)

(21) Appl. No.: 18/276,199

(22) PCT Filed: Feb. 11, 2022
(86) PCT No.: PCT/EP2022/053369

§ 371 Date: Aug. 7, 2023

(30) Foreign Application Priority Data
Feb. 11, 2021 (EP) 21156693.0

(51) Int. Cl. C12N 15/82

(52) U.S. Cl. CPC C12N 15/8222; C12N 15/8207; C12N 2830/15

(57) ABSTRACT

The present invention relates to plant promoter sequences comprising a combination of a cis-regulatory element (CRE) and a core promoter element (CPE), which is able to provide synergistically increased expression levels of a nucleic acid molecule of interest expressed under the control of the promoter sequences. Furthermore, the present invention relates to a method for increasing the expression level of a nucleic acid molecule of interest in a plant cell. Provided is also a plant cell or a plant obtained or obtainable by the method according to the invention and the use of a nucleic acid molecule comprising or consisting of a promoter according to the invention for increasing the expression level of a nucleic acid molecule of interest in a plant.

### TECHNICAL FIELD

The present invention provides a new technology to significantly increase the expression of a nucleic acid molecule of interest such as a trait gene, in a plant. In particular, the invention relates to plant promoter sequences comprising a combination of a cis-regulatory element (CRE) and a core promoter element (CPE), which is able to provide synergistically increased expression levels of a nucleic acid molecule of interest expressed under the control of the promoter sequences. Furthermore, the present invention relates to a method for increasing the expression level of a nucleic acid molecule of interest in a plant cell comprising introducing a modification at a first location in the original promoter of the nucleic acid molecule of interest to form a CRE and introducing another modification in a second location of the native promoter to form a CPE or, alternatively, replacing the original promoter of the nucleic acid molecule of interest with a promoter sequence according to the invention. The method optionally includes culturing at least one plant cell carrying the modifications or the substituted promoter sequence to obtain a plant showing an increased expression level of the nucleic acid molecule of interest. Further provided is a plant cell or a plant obtained or obtainable by the method according to the invention and the use of a nucleic acid molecule comprising or consisting of a promoter according to the invention for increasing the expression level of a nucleic acid molecule of interest in a plant.

### BACKGROUND

The expression levels of many genes in an organism depend on different factors such as developmental stages or physiologic and environmental conditions. The expression of one gene can be induced under certain circumstances and completely shut down if the circumstances change. The starting point for gene expression, the transcription of a gene, is regulated by a range of different mechanisms, which usually involve the promoter region harboring the transcription start site (TSS). While some promoters are active in all circumstances (constitutive promoters), others are tightly regulated and only respond to certain stimuli. Transcription factors bind to specific DNA sequences and activate or repress transcription (trans-acting factors). Promoter sequences therefore carry a number of binding sites for trans-acting factors also known as cis-regulatory elements. The core promoter region comprises the transcription start site as well as certain core promoter elements, including the TATA box motif, Y-patch motif, the initiator element and the downstream promoter element.

Being able to modulate the expression of certain genes in an organism opens up a range of opportunities to improve biotechnological processes or agricultural yields. Therefore, new technologies are continuously sought, which allow to specifically control expression levels of a target gene.

It has been shown that relatively short DNA sequences may provide enhancer activity on gene expression when present within a certain range of the promoter. For example, a 16 base pair palindromic sequence in the ocs element, was found to be essential for activity of the octopine synthase enhancer (Ellis et al., The EMBO Journal, 1987, Vol. 6, No. 11, pp. 3203-3208; Ellis et al., The Plant Journal, 1993, 4(3), 433-443).

Crop traits can be improved by increased expression of a trait gene (e.g., of the HPPD gene for herbicide resistance, or cell wall invertase genes for increased yield and drought tolerance). Usually, increased expression is achieved by transgenic approaches where these genes are ectopically expressed under control of strong constitutive promoters. However, transgenic approaches have the limitation that they result in high costs for deregulation and have low consumer acceptance.

A non-transgenic alternative to achieve increased expression of trait genes is the insertion of small expression modulating elements (WO2018183878A1) or promoter activating elements (WO2019185609A1) into the trait gene promoters. These small changes are usually in the range of 1 to 20 base pairs and can be achieved by targeted insertion (e.g., using site-directed nucleases and a small repair template) or by base editing/prime editing. A limitation of these current approaches is that the effect which can be achieved with an individual element always depends on the target gene.

It was an object of the present invention to provide means and methods for significantly increasing the expression of a nucleic acid sequence of interest in a plant. The method should be broadly applicable for different target sequences and in different plants. Preferably, the method to increase the expression of a target sequence should only require minimal modifications, i.e., of less than 30 nucleotides, preferably less than 20 nucleotides, of a given endogenous or heterologous sequence.

The present invention presents a significant improvement to the strategies mentioned above. It was found out that creating a combination of a cis-regulatory element (CRE) and a core promoter element (CPE) in optimal positions in the promoter results in synergistic effects, leading to a much stronger activation compared to what can be achieved with cis-regulatory or core promoter elements alone. Therefore, the new approach presented herein is more generic and more effective. Moreover, it is possible to introduce both elements by only minimal modification of a native promoter of a gene of interest and thus avoid the transgenic approaches. On the other hand, also the expression of transgenes can be enhanced with the technology presented herein. The presence of the CRE also allows a specific modulation of expression, e.g., stress-induced or tissue specific.

### SUMMARY OF INVENTION

In one aspect, the present invention relates to a method for increasing the expression level of a nucleic acid molecule of interest in a plant cell, the method comprising
(i) introducing a modification in the nucleic acid sequence of an endogenous promoter controlling the expression of the nucleic acid molecule of interest in a first location so that a cis-regulatory element selected from an as1-like element, a G-box element, a double G-box element, a TEF-box promoter motif, a corn CYP promoter fragment and a corn adh1 promoter element is formed and in a second location so that a core promoter element selected from a TATA box motif, a Y-patch motif, an initiator element and a downstream promoter element is formed, and
(ii) obtaining at least one plant cell showing an increased expression level of the nucleic acid molecule of interest compared to the expression level of the nucleic acid molecule of interest under the control of the unmodified endogenous promoter,
(iii) optionally, culturing the at least one plant cell obtained in step (ii) to obtain a plant showing an increased expression level of the nucleic acid molecule of interest compared to the expression level of the nucleic acid molecule of interest under the control of the unmodified endogenous promoter,
   wherein the first location is located upstream of the second location and the first and the second location are located at a distance of 5 to 225 nucleotides from each other, preferably at a distance of 10 to 160 nucleotides.

In one embodiment of the method described above, a second location is identified at a position −300 to −60 nucleotides relative to the start codon of the nucleic acid molecule of interest.

In another embodiment of the method described above, at least one of the first and the second location is located downstream of the transcription start site.

In one embodiment of the method described above, in step (i) less than 30 nucleotides are inserted, deleted and/or substituted at the first and/or the second location, preferably less than 25 nucleotides, preferably less than 20 nucleotides, preferably less than 15 nucleotides.

In another embodiment of the method described above, the modification in the first and/or second location is introduced by mutagenesis or by site-specific modification techniques using a site-specific nuclease or an active fragment thereof and/or a base editor and/or a prime editor.

In one embodiment of the method described above, step (i) comprises introducing into the cell a site-specific nuclease or an active fragment thereof, or providing the sequence encoding the same, the site-specific nuclease inducing a single- or double-strand break at a predetermined location, preferably wherein the site-specific nuclease or the active fragment thereof comprises a zinc-finger nuclease, a transcription activator-like effector nuclease, a CRISPR/Cas system, including a CRISPR/Cas9 system, a CRISPR/Cpf1 system, a CRISPR/C2C2 system a CRISPR/CasX system, a CRISPR/CasY system, a CRISPR/Cmr system, a CRISPR/MAD7 system, a CRISPR/CasZ system, an engineered homing endonuclease, a recombinase, a transposase and a meganuclease, and/or any combination, variant, or catalytically active fragment thereof; and optionally when the site-specific nuclease or the active fragment thereof is a CRISPR nuclease: providing at least one guide RNA or at least one guide RNA system, or a nucleic acid encoding the same; and optionally providing at least one repair template nucleic acid sequence.

In another embodiment of the method described above, the core promoter element is a TATA box motif having the sequence of CTATAAATA.

In one embodiment of the method described above, the cis-regulatory element is selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or has a sequence being 95%, 96%, 97%, 98% or 99% identical to any of the sequences of SEQ ID NOs: 5 to 8 or 198 to 202.

In another embodiment of the method described above, the first and the second location are located at a distance of 15 to 60 nucleotides from each other.

In one embodiment of the method described above, the expression level of the nucleic acid of interest controlled by the modified endogenous promoter is increased at least 20-fold, increased at least 50-fold, increased at least 100-fold, increased at least 150-fold, increased at least 200-fold, increased at least 250-fold, increased at least 300-fold, increased at least 350-fold, increased at least 400-fold in comparison to the expression level of the nucleic acid molecule of interest under the control of the unmodified endogenous promoter.

In one aspect, the present invention relates to a promoter, which is endogenous to a plant cell and which has been modified to provide an increased expression level of a nucleic acid molecule of interest in a plant cell, wherein the promoter has been modified to comprise
(a) a cis-regulatory element, which is heterologous to the promoter, selected from an as1-like element, a G-box element, a double G-box element, a TEF-box promoter motif, a corn CYP promoter fragment and a corn adh1 promoter element, and
(b) a TATA box motif having the sequence of CTATAAATA and being heterologous to the promoter,
wherein the cis-regulatory element is located upstream of the TATA box motif and the cis-regulatory element and the TATA box motif are positioned at a distance of 5 to 225 nucleotides from each other, preferably positioned at a distance of 10 to 160 nucleotides from each other, and wherein the expression level provided by the endogenous modified promoter is increased synergistically with respect to the endogenous promoter comprising only said cis-regulatory element or said TATA box motif sequence.

In one embodiment of the promoter described above, at least one of the cis-regulatory element and the TATA box motif are located downstream of the transcription start site.

In another embodiment of the promoter described above, the modified promoter provides an increased expression level of a nucleic acid molecule of interest compared to the expression level of a nucleic acid molecule of interest under the control of the unmodified endogenous promoter.

In one embodiment of the promoter described above, the cis-regulatory element and the TATA box motif are located at a distance of 15 to 60 nucleotides from each other.

In another embodiment of the promoter described above, the expression level of an nucleic acid of interest controlled by the modified endogenous promoter is increased at least 20-fold, increased at least 50-fold, increased at least 100-fold, increased at least 150-fold, increased at least 200-fold, increased at least 250-fold, increased at least 300-fold, increased at least 350-fold, increased at least 400-fold in comparison to the expression level of the nucleic acid molecule of interest under the control of the unmodified endogenous promoter.

In one embodiment of the promoter described above, the cis-regulatory element is selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or has a sequence being 95%, 96%, 97%, 98% or 99% identical to any of the sequences of SEQ ID NOs:5 to 8 or 198 to 202.

In a further aspect, the present invention relates to a nucleic acid molecule comprising or consisting of a promoter sequence, which is endogenous to a plant cell and which has been modified to comprise
(a) a cis-regulatory element selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or having a sequence being 95%, 96%, 97%, 98% or 99% identical to any of the sequences of SEQ ID NOs: 5 to 8 or 198 to 202, and
(b) a TATA box motif having the sequence of CTATAAATA, located at a position −300 to −60 nucleotides relative to the start codon,
wherein (a) and (b) are located at a distance of 15 to 60 nucleotides to each other,
and wherein the expression level provided by the modified endogenous promoter is increased at least 20-fold with respect to a promoter comprising no modification and wherein the expression level provided by the promoter is increased synergistically with respect to an endogenous promoter comprising only said cis-regulatory element or said TATA box motif.

In one embodiment of the nucleic acid molecule described above, at least one of the cis-regulatory element and the core promoter element are located downstream of the transcription start site.

In another aspect, the present invention relates to a plant cell or a plant obtained or obtainable by a method according to any of the embodiments described above.

In yet another aspect, the present invention relates to the use of a nucleic acid molecule according to any of the embodiments described above, or the use of a modified promoter according to any of the embodiments described above for increasing the expression level of a nucleic acid molecule of interest in a plant cell, preferably in a method according to any of the embodiments described above.

In one aspect, the present invention relates to a nucleic acid molecule comprising or consisting of a promoter active in a plant cell, comprising
(a) a cis-regulatory element, and
(b) a core promoter element,
wherein the cis-regulatory element is located upstream of the core promoter element and the cis-regulatory element, and the core promoter element are located at a distance of 5 to 225 nucleotides from each other, preferably 10 to 160 nucleotides, particularly preferably 15 to 60 nucleotides, and wherein the expression level provided by the promoter is increased synergistically with respect to a promoter comprising only one of the cis-regulatory element and the core promoter element.

In one embodiment of the nucleic acid molecule described above, the core promoter element is located at a position −300 to −60 nucleotides relative to the start codon.

In an embodiment of the nucleic acid molecule according to any of the embodiments described above, at least one of the cis-regulatory element and the core promoter element is located downstream of the transcription start site.

In one embodiment of the nucleic acid molecule according to any of the embodiments described above, the cis-regulatory element is selected from an as1-like element, a G-box element, a double G-box element, a TEF-box promoter motif, a corn CYP promoter fragment and a corn adh1 promoter element.

In another embodiment of the nucleic acid molecule according to any of the embodiments described above, the core promoter element is selected from a TATA box motif, a Y-patch motif, an initiator element and a downstream promoter element.

In another aspect, the present invention relates to a method for increasing the expression level of a nucleic acid molecule of interest in a plant cell, the method comprising
(i) introducing a modification in the nucleic acid sequence of the original promoter controlling the expression of the nucleic acid molecule of interest in a first location so that a cis-regulatory element is formed and in a second location so that a core promoter element is formed, or introducing a nucleic acid molecule as defined in any of the embodiments described above replacing the original promoter, and
(ii) obtaining at least one plant cell showing an increased expression level of the nucleic acid molecule of interest compared to the expression level of the nucleic acid molecule of interest under the control of the unmodified original promoter, and
(iii) optionally, culturing the at least one plant cell obtained in step (ii) to obtain a plant showing an increased expression level of the nucleic acid molecule of interest compared to the expression level of the nucleic acid molecule of interest under the control of the unmodified original promoter,
   wherein the first location is located upstream of the second location and the first and the second location are located at a distance of 5 to 225 nucleotides from each other, preferably 10 to 160 nucleotides, particularly preferably 15 to 60 nucleotides.

In one embodiment of the method described above, a second location is identified at a position −300 to −60 nucleotides relative to the start codon of the nucleic acid molecule of interest and, subsequently the first location is determined at a suitable distance upstream of the second location.

In another embodiment of the method according to any of the embodiments described above, at least one of the first and the second location is located downstream of the transcription start site.

In another embodiment of the method according to any of the embodiments described above, in step (i) less than 30 nucleotides are inserted, deleted and/or substituted at the first and/or the second location, preferably less than 25 nucleotides, preferably less than 20 nucleotides, preferably less than 15 nucleotides.

In one embodiment of the method according to any of the embodiments described above, the cis-regulatory element is selected from an as1-like element, a G-box element, a double G-box element, a TEF-box promoter motif, a corn CYP promoter fragment and a corn adh1 promoter element.

In another embodiment of the method according to any of the embodiments described above, the core promoter element is selected from a TATA box motif, a Y-patch motif, an initiator element and downstream promoter element.

In one embodiment of the method according to any of the embodiments described above, the modification in the first and/or second location is introduced by mutagenesis or by site-specific modification techniques using a site-specific nuclease or an active fragment thereof and/or a base editor and/or a prime editor.

In another embodiment of the method according to any of the embodiments described above, step (i) comprises introducing into the cell a site-specific nuclease or an active fragment thereof, or providing the sequence encoding the same, the site-specific nuclease inducing a single- or double-strand break at a predetermined location, preferably wherein the site-specific nuclease or the active fragment thereof comprises a zinc-finger nuclease, a transcription activator-like effector nuclease, a CRISPR/Cas system, including a CRISPR/Cas9 system, a CRISPR/Cpf1 system, a CRISPR/C2C2 system a CRISPR/CasX system, a CRISPR/CasY system, a CRISPR/Cmr system, a CRISPR/MAD7 system, a CRISPR/CasZ system, an engineered homing endonuclease, a recombinase, a transposase and a meganuclease, and/or any combination, variant, or catalytically active fragment thereof; and optionally when the site-specific nuclease or the active fragment thereof is a CRISPR nuclease: providing at least one guide RNA or at least one guide RNA system, or a nucleic acid encoding the same; and optionally providing at least one repair template nucleic acid sequence.

In yet another embodiment of the method according to any of the embodiments described above, the expression level of the nucleic acid molecule of interest is increased synergistically with respect to the modification introduced only at the first or the second location.

In another aspect, the present invention relates to a plant cell, or a plant obtained or obtainable by a method according to any of the embodiments described above.

In yet another aspect, the present invention relates to the use of a nucleic acid molecule according to any of the embodiments described above for increasing the expression level of a nucleic acid molecule of interest in a plant cell, preferably in a method according to any of the embodiments described above.

### Definitions

A “promoter” or a “promoter sequence” refers to a DNA sequence capable of controlling and/or regulating expression of a coding sequence, i.e., a gene or part thereof, or of a functional RNA, i.e., an RNA which is active without being translated, for example, a miRNA, a siRNA, an inverted repeat RNA or a hairpin forming RNA. A promoter is located at the 5′ part of the coding sequence. Promoters can have a broad spectrum of activity, but they can also have tissue or developmental stage specific activity. For example, they can be active in cells of roots, seeds and meristematic cells, etc. A promoter can be active in a constitutive way, or it can be inducible. The induction can be stimulated by a variety of environmental conditions and stimuli. Often, promoters are highly regulated. A promoter of the present disclosure may include an endogenous promoter natively present in a cell, or an artificial or transgenic promoter, either from another species, or an artificial or chimeric promoter, i.e., a promoter that does not occur in nature in this composition and is composed of different promoter elements. In the context of the present disclosure, a promoter or promoter sequence comprises the transcription start site (TSS) as well as regulatory elements such as cis-regulatory elements (CREs) and core promoter elements (CPEs) upstream and downstream of the TSS. The promoter or promoter sequence extends downstream of the TSS including the 5′-untranslated region (5′-UTR) and ending at the position −1 relative to the start codon of the coding sequence, which is expressed under the control of the promoter.

The “start codon” represents the first codon of a sequence, which is translated into an amino acid during expression of a coding sequence. The most common start codon is ATG, which is translated into methionine. In the context of the present disclosure, the start codon refers to the first three nucleotides of the nucleic acid molecule, which is expressed under the control of the promoter of the invention, or of the nucleic acid molecule of interest. If the nucleic acid molecule to be expressed under the control of the promoter is a functional RNA, the start codon is not translated into protein but merely transcribed into RNA.

The term “gene expression” or “expression” as used herein refers to the conversion of the information, contained in a gene or nucleic acid molecule, into a “gene product” or “expression product”. A “gene product” or “expression product” can be the direct transcriptional product of a gene or nucleic acid molecule (e.g., mRNA, tRNA, rRNA, antisense RNA, ribozyme, structural RNA or any other type of RNA) or a protein produced by translation of an mRNA. Gene products or expression products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

An “expression level provided by a promoter” is “increased” when the expression level of the nucleic acid molecule under the control of the promoter is higher when the promoter comprises a cis-regulatory element and a core promoter element as defined herein compared to the expression level of the nucleic acid molecule under the control of the promoter not comprising the cis-regulatory element and the core promoter element. The expression level is “synergistically increased” when the expression level of the nucleic acid molecule under the control of the promoter comprising a cis-regulatory element and a core promoter element as defined herein is higher than the sum of the expression levels observed when the promoter only comprises either the cis-regulatory element or the core promoter element.

A “cis-regulatory element” or “CRE” is a non-coding DNA sequence located in the promoter, which regulates the transcription of the gene under the control of the promoter. Cis-regulatory elements represent binding sites for trans-acting factors such as transcription factors. In the context of the present disclosure, a cis-regulatory element is a sequence, which functions as an enhancer of expression when it is present within a certain range of the start codon of a gene of interest and a cis-regulatory element is not a core promoter element as defined below. In particular, a cis-regulatory element is an as1-like element or a (double) G-box element.

An “as1 element” or “activation sequence 1 (as1)” is a binding site for the activation sequence factor 1 (ASF1) found in the 35S promoter of cauliflower mosaic virus (Lam et a., Site-specific mutations alter in vitro factor binding and change promoter expression pattern in transgenic plants, Proc. Natl. Acad. Sci. USA, 1989, Vol. 86, pp. 7890-7894). As1-like elements also cover similar sequences from other organisms. In the context of the present disclosure, an as1-like element comprises at least one TKACG motif, wherein K stands for G or T, preferably K stands for G. Further preferably, it comprises two TKACG motifs, which may be in tandem or separated by one or more nucleotides. An as1-like element is therefore characterized by the consensus sequence TKACG or by the consensus sequence TKACGNTKACG (SEQ ID NO: 1), wherein wherein N stands for 0, 1, 2, 3, 4, 5, 6, 7, 8, 9, 10 or up to 15, up to 20, up to 25, up to 30, up to 35, up to 40, up to 45 or up to 50 arbitrary nucleotide(s).

The G-box represents a binding site for the G-box binding factor (GBF) (Donald et al., The plant G box promoter sequence activates transcription in Saccharomyces cerevisiae and is bound in vitro by a yeast activity similar to GBF, the plant G box binding factor, The EMBO Journal, 1990, Vol. 9, No. 6, 1727-1735). A “G-box element” is characterized by a CACGTG motif and a “double G-box element” is characterized by two CACGTG motifs, which may be in tandem or separated by one or more nucleotides. A G-box element is characterized by the consensus sequence CACGTG, while a double G-box element is characterized by the consensus sequence CACGTGNCACGTG (SEQ ID NO: 2), wherein N stands for 0, 1, 2, 3, 4, 5, 6, 7, 8, 9, 10 or up to 15, up to 20, up to 25, up to 30, up to 35, up to 40, up to 45 or up to 50 arbitrary nucleotide(s).

A “TEF-box promoter motif” is characterized by the consensus sequence ARGGRYANNNNNGT (SEQ ID NO: 221), wherein R stands for A or G, Y stands for C or T and N stands for A, C, G or T. A preferred consensus sequence is AGGGGCATAATGGT (SEQ ID NO: 222) (Trémousaygue et al., Internal telomeric repeats and ‘TCP domain’ protein-binding sites co-operate to regulate gene expression in Arabidopsis thaliana cycling cells, Plant J., 2003 March; 33(6): 957-66. doi: 10.1046/j.1365-313x.2003.01682.x.)

A “corn CYP promoter fragment” is characterized by the consensus sequence ACACNNG, wherein N stands for A, C, G or T (DPBFCOREDCDC3). A preferred consensus sequence is ACACAGG (Kim et al., Isolation of a novel class of bZIP transcription factors that interact with ABA-responsive and embryo-specification elements in the Dc3 promoter using a modified yeast one-hybrid system, Plant J., 1997 June; 11(6): 1237-51. doi: 10.1046/j.1365-313x.1997.11061237.x.).

A “corn adh1 promoter element” is characterized by the hexamer motif ACGTCA found in promoter of wheat histone genes (Mikami et al., Wheat nuclear protein HBP-1 binds to the hexameric sequence in the promoter of various plant genes, Nucleic Acids Res. 1989 Dec. 11; 17(23): 9707-17. doi: 10.1093/nar/17.23.9707.).

A “core promoter” or “core promoter sequence” refers to a part of a promoter, which is necessary to initiate the transcription and comprises the transcription start site (TSS). A “core promoter element” or “CPE” is a sequence present in the core promoter such as a TATA box motif, a Y-patch motif, an initiator element and a downstream promoter element. A core promoter element can be identified by a consensus sequence, which is defined by one or more conserved motifs.

A “TATA box motif” refers to a sequence found in many core promoter regions of eukaryotes. The native TATA box motif is usually found within 100 nucleotides upstream of the transcription start site. In plant promoters, the native TATA-box motif is found about 25 to 40 nt, preferably 31 to 32 nt, upstream of the transcription start site. The TATA box motif also represents the binding site for TBP (TATA box binding protein). The “TATA box consensus sequences” is CTATAWAWA, wherein W stand for A or T. An ideal TATA box motif is represented by CTATAAATA.

A “Y-patch motif” or “Y-patch promoter element” or “pyrimidine patch promoter element” or “Y-patch” or “pyrimidine patch” refers to a sequence found in many promoters of higher plants. A typical Y-patch is composed of C and T (pyrimidine) (Yamamoto et al., Nucleic Acids Research, 2007, Differentiation of core promoter architecture between plants and mammals revealed by LDSS analysis, 35(18): 6219-26). A Y-patch can be detected by LDSS (local distribution of short sequences) analysis as well as by a search for consensus sequence from plant promotors, preferably core promoters, by MEME and AlignACE (Molina & Grotewold. Genome wide analysis of Arabidopsis core promoters. BMC Genomics. 2005; 6:25). Y-patches are often found downstream of the transcription start site. The consensus sequence for the Y-patch is given in CYYYYYYYYC (SEQ ID NO: 3), wherein Y stands for C or T. An exemplary sequence is given in CCTCCTCCTC (SEQ ID NO: 4), SEQ ID NO: 203 and SEQ ID NO: 204.

An “initiator element (Inr)” is a core promoter sequence, which has a similar function as the TATA box and can also enable transcription initiation in the absence of a TATA box. It facilitates the binding of transcription factor II D, which is part of the RNA polymerase II preinitiation complex. The Inr encompasses the TSS and may contain a dimer motif (C/T A/G).

A “downstream promoter element (DPE)” is a core promoter sequence, which also plays a role in transcription initiation and is located downstream of the TSS. It is recognized by the transcription factor II D together with the Inr.

A “nucleic acid molecule of interest” refers to any coding sequence, which is transcribed and/or translated into a gene product or an expression product in a plant. It can either refer to a functional RNA or a protein. In particular, the nucleic acid molecule of interest may be a trait gene, which is desired to be expressed at a high level at any time or under certain conditions. Preferably, the nucleic acid molecule of interest provides or contributes to agricultural traits such as biotic or abiotic stress tolerance or yield related traits.

In all aspects and embodiments of the present invention, an optimal distance between the cis-regulatory element and the core promoter element is a distance of 5 to 225 nucleotides, preferably 10 to 160 nucleotides, particularly preferably 15 to 60 nucleotides. This means that a maximum of 225, 160 or 60 nucleotides and a minimum of 5, 10 or 15 nucleotides is present between the cis-regulatory element and the core promoter element once they are formed/introduced in the promoter sequence.

The “original promoter controlling the expression of the nucleic acid molecule of interest” is the promoter, which is controlling the expression of the nucleic acid molecule of interest before the modifications or the replacement according to the invention are implemented. The original promoter may be a native promoter naturally controlling the expression of the nucleic acid molecule of interest in the plant or it may be a non-native promoter, which has been introduced into the plant by genome engineering or introgression, optionally together with the nucleic acid molecule of interest. The original promoter may be endogenous to the plant it is active in, or it may be exogenous, i.e., derived from a different organism. It may be a synthetic, recombinant or artificial promoter, which does not occur in nature. Moreover, it can be heterologous in respect to the gene, the expression of which it controls. It may also be a transgenic, inserted, modified or mutagenized promoter. In the method for increasing the expression level of a nucleic acid molecule of interest in a plant cell according to the invention, the unmodified original promoter present before the introduction of the modification(s) represents the control for determining an increase of expression level. To this end, the nucleic acid molecule of interest is expressed under the same conditions (environmental conditions, developmental stage etc.) under the control of the unmodified original promoter and under the control of the modified promoter and the expression levels are compared in a suitable manner.

“Endogenous” in the context of the present disclosure means that a certain sequence or sequence motif is native to a cell or an organism, i.e. it naturally occurs in this cell or organism. A sequence or sequence motif can also be endogenous to another sequence meaning that it naturally forms a part of this sequence. “Heterologous”, on the other hand, means that a certain sequence or sequence motif does not naturally occur in a certain context, e.g. in a certain cell or an organism or within (as part of) a certain sequence. A heterologous sequence or sequence motif is introduced by sequence modification.

“Modifying a (nucleic acid) sequence” or “introducing a modification into a nucleic acid sequence” in the context of the present invention refers to any change of a (nucleic acid) sequence that results in at least one difference in the (nucleic acid) sequence distinguishing it from the original sequence. In particular, a modification can be achieved by insertion or addition of one or more nucleotide(s), or substitution or deletion of one or more nucleotide(s) of the original sequence or any combination of these. “Addition” refers to one or more nucleotides being added to a nucleic acid sequence, which may be contiguous or single nucleotides added at one or more positions within the nucleic acid sequence. “Substitution” refers to the exchange of one or more nucleotide(s) of a nucleic acid sequence by one or more different nucleotide(s). A substitution may be a replacement of one or more nucleotide(s) or a modification of one or more nucleotide(s) that results in (a) different nucleotide(s) e.g., by conversion of a nucleobase to a different nucleobase. A nucleic acid sequence of a certain length may also be replaced as a whole with another sequence of the same or a different length. “Deletion” refers to the removal of one or more nucleotide(s) from a nucleic acid sequence.

“Mutagenesis” refers to a technique, by which modifications or mutations are introduced into a nucleic acid sequence in a random or non-site-specific way. For example, mutations can be induced by certain chemicals such as EMS (ethyl methanesulfonate) or ENU (Nethyl-N-nitrosourea) or physically, e.g., by irradiation with UV or gamma rays. “Site-specific modifications”, on the other hand, rely on the action of site-specific effectors such as nucleases, nickases, recombinases, transposases, base editors. These tools recognize a certain target sequence and allow to introduce a modification at a specific location within the target sequence.

A “site-specific nuclease” refers to a nuclease or an active fragment thereof, which is capable to specifically recognize and cleave DNA at a certain location. This location is herein also referred to as a “predetermined location”. Such nucleases typically produce a double strand break (DSB), which is then repaired by nonhomologous end-joining (NHEJ) or homologous recombination (HR). The nucleases include zinc-finger nucleases, transcription activator-like effector nucleases, CRISPR/Cas systems, including CRISPR/Cas9 systems, CRISPR/Cpf1 systems, CRISPR/C2C2 systems, CRISPR/CasX systems, CRISPR/CasY systems, CRISPR/Cmr systems, CRISPR/MAD7 systems, CRISPR/CasZ systems, engineered homing endonucleases, recombinases, transposases and meganucleases, and/or any combination, variant, or catalytically active fragment thereof.

A “CRISPR nuclease”, as used herein, is any nuclease which has been identified in a naturally occurring CRISPR system, which has subsequently been isolated from its natural context, and which preferably has been modified or combined into a recombinant construct of interest to be suitable as tool for targeted genome engineering. Any CRISPR nuclease can be used and optionally reprogrammed or additionally mutated to be suitable for the various embodiments according to the present invention as long as the original wild-type CRISPR nuclease provides for DNA recognition, i.e., binding properties. Said DNA recognition can be PAM (protospacer adjacent motif) dependent. CRISPR nucleases having optimized, and engineered PAM recognition patterns can be used and created for a specific application. The expansion of the PAM recognition code can be suitable to target site-specific effector complexes to a target site of interest, independent of the original PAM specificity of the wild-type CRISPR-based nuclease. Cpf1 variants can comprise at least one of a S542R, K548V, N552R, or K607R mutation, preferably mutation S542R/K607R or S542R/K548V/N552R in AsCpf1 from Acidaminococcus. Furthermore, modified Cas or Cpf1 variants or any other modified CRISPR effector variants, e.g., Cas9 variants, can be used according to the methods of the present invention as part of a base editing complex, e.g., BE3, VQR-BE3, EQR-BE3, VRER-BE3, SaBE3, SaKKH-BE3 (see Kim et al., Nat. Biotech., 2017, doi:10.1038/nbt.3803). Therefore, according to the present invention, artificially modified CRISPR nucleases are envisaged, which might indeed not be any “nucleases” in the sense of double-strand cleaving enzymes, but which are nickases or nuclease-dead variants, which still have inherent DNA recognition and thus binding ability. Suitable Cpf1-based effectors for use in the methods of the present invention are derived from Lachnospiraceae bacterium (LbCpf1, e.g., NCBI Reference Sequence: WP_051666128.1), or from Francisella tularensis (FnCpf1, e.g., UniProtKB/Swiss-Prot: AOQ7Q2.1). Variants of Cpf1 are known (cf. Gao et al., BioRxiv, dx.doi.org/10.1101/091611). Variants of AsCpf1 with the mutations S542R/K607R and S542R/K548V/N552R that can cleave target sites with TYCV/CCCC and TATV PAMs, respectively, with enhanced activities in vitro and in vivo are thus envisaged as site-specific effectors according to the present invention. Genome-wide assessment of off-target activity indicated that these variants retain a high level of DNA targeting specificity, which can be further improved by introducing mutations in non-PAM-interacting domains. Together, these variants increase the targeting range of AsCpf1 to one cleavage site for every ˜8.7 base pairs (bp) in non-repetitive regions of the human genome, providing a useful addition to the CRISPR/Cas genome engineering toolbox (see Gao et al., supra).

A “base editor” as used herein refers to a protein or a fragment thereof having the same catalytic activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest. Preferably, the at least one base editor in the context of the present invention is temporarily or permanently linked to at least one site-specific effector, or optionally to a component of at least one site-specific effector complex. The linkage can be covalent and/or non-covalent. Base editors, as understood herein including BEs (base editors mediating C to T conversion) and ABEs (adenine base editors mediating A to G conversion), are powerful tools to introduce direct and programmable mutations of all four transitions to the DNA without the need for double-stranded cleavage (Komor et al., 2016, Programmable editing of a target base in genomic NDA without double-stranded DNA cleavage, Nature, 533, 420-424; Gaudelli et al. 2017. Programmable base editing of A·T to G·C in genomic DNA without DNA cleavage. Nature, 551(7681), 464). In general, base editors are composed of at least a DNA targeting module and a catalytic domain that deaminates cytidine or adenine. Both BEs and ABEs are originally developed by David Liu's lab. There are three BE versions described in Komor et al., 2016, namely BE1, BE2 and BE3, with BE3 showing the highest efficiency of targeted C to T conversion, resulting in up to 37% of desired C to T conversion in human cells. BE3 is composed of APOBEC-XTEN-dCas9 (A840H)-UGI, where APOBEC1 is a cytidine deaminase, XTEN is 16-residue linker, dCas9 (A840H) is a nickase version of Cas9 that nicks the non-edited strand and UGI is an Uracil DNA glycosylase inhibitor. In this system, the BE complex is guided to the target DNA by the sgRNA, where the cytosine is then converted to uracil by cytosine deamination. The UGI inhibits the function of cellular uracil DNA glycosylase, which catalyses removal of uracil from DNA and initiates base-excision repair (BER). And the nicking of the unedited DNA strand helps to resolve the U:G mismatch into desired U:A and T:A products. As mentioned above, BEs are efficient in converting C to T (G to A) but are not capable for A to G (T to C) conversion. ABEs were first developed by Gaudelli et al., for converting A-T to G-C. A transfer RNA adenosine deaminase was evolved to operate on DNA, which catalyzes the deamination of adenosine to yield inosine, which is read and replicated as G by polymerases. By fusion of the evolved adenine deaminase and a Cas9 module, ABEs described in Gaudelli et al., 2017 showed about 50% efficiency in targeted A to G conversion. All four transitions of DNA (A-T to G-C and C-G to T-A) are possible as long as the base editors can be guided to the target place. Base editors convert C or A at the non-targeted strand of the sgRNA. Originally developed for working in mammalian cell systems, both BE and ABEs have been optimized and applied in plant cell systems. Efficient base editing has been shown in multiple plant species (Zong et al., 2017, Precise base editing in rice, wheat and maize with a Cas9-cytidine deaminase fusion, Nat. Biotechnology, 35(5), 438-440; Yan et al., 2018, Highly efficient A-T to G-C Base Editing by Cas9n-Guided tRNA Adenosine Deaminase in Rice, Molecular Plant, Vol. 11, Issue 4, 631-634; Hua et al., 2018, Precise A-Tto G-C Base Editing in the Rice Genome, Mol. Plant, 11(4), 627-630). Zong et al., adopted the BE2 and BE3 (Komor et al., 2016), which are composed of ratAPOBEC1-Cas9 (catalytically dead for BE2 and nickase for BE3)-UGI, codon optimized the sequence for cereal plants, cloned them under the maize Ubiquitin-1 gene promoter and then applied them in rice, wheat and maize. They reported that using CRISPR-Cas9 nickase-cytidine deaminase fusion, the targeted conversion of C to T in both protoplasts and regenerated rice, wheat and maize plants showed frequencies up to 43.48%. Yan et al., 2018 and Hua et al., 2018 both reported the adoption of ABE described in Gaudelli et al., 2017 to generate targeted A-T to G-C mutations in rice plants. Codon optimization for expression in rice was performed in Yan et al., 2018; whereas Hua et al., used the mammalian codon-optimized sequences described in Gaudelli et al., 2017 in addition with a strong VirD2 nuclear localization signal fusion to the C terminus of the Cas9 (D10A) nickase from both S. pyogenes and S. aureus. Both studies demonstrated successful application of ABEs that introduce A to G conversion in rice plants.

A “prime editor” as used herein refers to a system as disclosed in Anzalone et al. (Searchand-replace genome editing without double-strand breaks or donor DNA, Nature, 576, 149-157 (2019). Base editing as detailed above, does not cut the double-stranded DNA, but instead uses the CRISPR targeting machinery to shuttle an additional enzyme to a desired sequence, where it converts a single nucleotide into another. Many genetic traits in plants and certain susceptibility to diseases caused by plant pathogens are caused by a single nucleotide change, so base editing offers a powerful alternative for GE. But the method has intrinsic limitations and is said to introduce off-target mutations which are generally not desired for high precision GE. In contrast, Prime Editing (PE) systems steer around the shortcomings of earlier CRISPR based GE techniques by heavily modifying the Cas9 protein and the guide RNA. The altered Cas9 only “nicks” a single strand of the double helix, instead of cutting both. The new guide RNA, called a pegRNA (prime editing extended guide RNA), contains an RNA template for a new DNA sequence, to be added to the genome at the target location. That requires a second protein, attached to Cas9 or a different CRISPR effector nuclease: a reverse transcriptase enzyme, which can make a new DNA strand from the RNA template and insert it at the nicked site. To this end, an additional level of specificity is introduced into the GE system in view of the fact that a further step of target specific nucleic acid::nucleic acid hybridization is required. This may significantly reduce off-target effects. Further, the PE system may significantly increase the targeting range of a respective GE system in view of the fact that BEs cannot cover all intended nucleotide transitions/mutations (C→A, C→G, G→C, G→T, A→C, A→T, T→A, and T→G) due to the very nature of the respective systems, and the transitions as supported by BEs may require DSBs in many cell types and organisms.

Whenever nucleic acid sequences are given in the context of the present disclosure, N stands for any of A, C, T or G; W stands for A or T; S stands for C or G; M stands for A or C; K stands for G or T; R stands for A or G; Y stands for C or T; B stands for C, G or T; D stands for A, G or T; H stands for A, C or T and V stand for A, C or G.

Whenever the present disclosure relates to the percentage of identity of nucleic acid or amino acid sequences to each other these values define those values as obtained by using the EMBOSS Water Pairwise Sequence Alignments (nucleotide) program or the EMBOSS Water Pairwise Sequence Alignments (protein) program (www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see www.ebi.ac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5. The skilled person is well aware of the fact that, for example, a sequence encoding a protein can be “codon-optimized” if the respective sequence is to be used in another organism in comparison to the original organism a molecule originates from.

### BRIEF DESCRIPTION OF THE FIGURES

FIG. 1A: The upper part of the figure displays a sketch of the ZmCWI3 promoter with positions indicated. B: The graph shows the results from transient testing of the promoter modifications as promoter activity deduced from the respective luciferase measurement relative to the unmodified promoter (see Example 1). CWI3-control represents the unmodified promoter (SEQ ID NO: 184). In CWI3v2, an additional TATA box (CTATAAATA) was created by 4 point mutations at position v2 (SEQ ID NO: 185). In CWI3v3-2, the endogenous TATA box (CTACAAATA) was optimized by a one point mutation to CTATAAATA (SEQ ID NO: 186). In CWI3-50-E039g, an as1-like CRE (E039g, SEQ ID NO: 5) was inserted at the −50 position, which is at a 37 bp distance to position v3-2 (SEQ ID NO: 187). The combination of the TATA box at position v2 and the CRE (E039g, SEQ ID NO: 5) at the −50 position (CWI3v2-50-E39g, SEQ ID NO: 188) did not result in an enhancement of expression because in this case the CRE is located downstream of the TATA box. With the combination CWI3v3-2-50-E039g (SEQ ID NO: 189), however, where the TATA box is at the v3-2 position, a synergistic enhancement is observed.

FIG. 2A: The upper part of the figure displays a sketch of the BvHPPD1 promoter with positions indicated. B: The graph shows the results from transient testing of the promoter modifications as promoter activity deduced from the respective luciferase measurement relative to the unmodified promoter (see Example 2). HPPD1-control represents the unmodified promoter (SEQ ID NO: 190). In HPPD1v3, an additional TATA box (CTATAAATA) was created by 4 point mutations at position v3, which is at a 66 bp distance from the −50 position (SEQ ID NO: 191). In HPPD1v4, an additional TATA box (CTATAAATA) was created by 3 point mutations at position v4, which is at a 106 bp distance from the −50 position (SEQ ID NO: 192). In HPPD1v5, an additional TATA box (CTATAAATA) was created by 5 point mutations at position v5, which is at a 151 bp distance from the −50 position (SEQ ID NO: 193). In HPPD1-50-E38f, an as1-like CRE (E038f, SEQ ID NO: 6) was inserted at the −50 position (SEQ ID NO: 194). The combination of the TATA box at position v3 and the CRE (E038f, SEQ ID NO: 6) at the −50 position (HPPD1v3-50-E38f, SEQ ID NO: 195) shows a synergistic enhancement of expression. The same is true for the combination of the TATA box at positions v4 and v5 with the CRE (E038f, SEQ ID NO: 6) at the −50 position (HPPD1v4-50-E38f and HPPD1v5-50-E38f, SEQ ID NOs: 196 and 197).

FIG. 3A: The upper part of the figure displays a sketch of the Bv-prom3 promoter with positions indicated. B: The graph shows the results from transient testing of the promoter modifications as promoter activity deduced from the respective luciferase measurement relative to the unmodified promoter (see Example 3). Bv-prom3-control represent the unmodified promoter. In Bv-prom3-50-E38h, an as1-like CRE (E038h, SEQ ID NO: 7) is inserted via element ligation at the −50 position, which is −362 bp upstream of the start codon. In Bv-prom3-50-E128, a double G-box CRE (E128, SEQ ID NO: 8) is inserted via element ligation at the −50 position, which is −362 bp upstream of the start codon. In Bv-prom3v3, an additional TATA-box (CTATAAATA) is generated by exchange of 4 bases. This additional TATA-box is positioned at −197 bp upstream of the start codon (position v3). In Bvprom3v4, an additional TATA-box (CTATAAATA) is generated by exchange of 5 bases. This additional TATA-box is positioned at −153 bp upstream of the start codon (position v4). A combination of E038h or E128 at the −50 position and an additional TATA box at position v3 (Bv-prom3v3-50-E038h and Bv-prom3v3-50-E128) results in a synergistic enhancement of expression. In these two promoters, the CRE and CPE are at a distance of 145 bp from each other. A combination of E038h and E128 at the −50 position and an additional TATA box at position v4 (Bv-prom3v4-50-E038h and Bv-prom3v4-50-E128), on the other hand, does not result in an enhancement of expression. In these two promoters, the CRE and CPR are at a distance of 189 bp from each other, indicating that there is a maximum distance for a synergism to occur.

FIG. 4A: The upper part of the figure displays a sketch of the BvHPPD1 promoter with positions indicated (same as FIG. 2A). B: The graph shows the results from transient testing of the promoter modifications. The promoter activity is deduced from the respective luciferase measurement relative to the unmodified promoter (see Example 4). HPPD1-control represents the unmodified promoter (SEQ ID NO: 190). The as1-like CRE E038f (SEQ ID NO: 6) and the double G-box CRE E133 (SEQ ID NO: 199) are inserted at the −50 position (SEQ ID NO: 194 and SEQ ID NO: 205). The combination of the TATA box at position v5 with the different types of CRE (E038f, SEQ ID NO: 6 or E133, SEQ ID NO: 199) at the −50 position leads to synergistic enhancement of expression (HPPD1v5-50-E38f, SEQ ID NO: 197 and HPPD1v5-50-E133, SEQ ID NO: 206).

FIG. 5A: The upper part of the figure displays a sketch of the BvHPPD2 promoter with positions indicated. B: The graph shows the results from transient testing of the promoter modifications. The promoter activity is deduced from the respective luciferase measurement relative to the unmodified promoter (see Example 5). HPPD2-control represents the unmodified promoter (SEQ ID NO: 207). The as1-like CRE E038h (SEQ ID NO: 7) and the double G-box CRE E128 (SEQ ID NO: 8) are inserted at the −50 position (SEQ ID NO: 209 and SEQ ID NO: 210). The combination of the TATA box at position v5 with the different types of CRE (E038h, SEQ ID NO: 7 or E133, SEQ ID NO: 8) at the −50 position leads to synergistic enhancement of expression (HPPD2v5-50-E38h, SEQ ID NO: 211 and HPPD2v5-50-E128, SEQ ID NO: 212).

FIG. 6A: The upper part of the Figure displays a sketch of the Zm-prom6 promoter with positions indicated. B: The graph shows the results from transient testing of the promoter modifications. The promoter activity is deduced from the respective luciferase measurement relative to the unmodified promoter (see Example 6). Zm-prom6 control represents the unmodified promoter. Different cis-regulatory elements (CRE), like the as1-like elements E039g (SEQ ID NO: 5) and E039i (SEQ ID NO: 198), the TEF-box promoter motif E016 (SEQ ID NO: 200), a corn CYP promoter fragment E101c (SEQ ID NO: 201) and the corn adh1 promoter element E115d (SEQ ID NO: 202) are inserted at the −125 position. The combination of an inserted TATA box at position v6a with the different types of CRE at the −125 position leads to synergistic enhancement of expression.

FIG. 7A: The upper part of the figure displays a sketch of the BvFT2 promoter with positions indicated. B: The graph shows the results from transient testing of the promoter modifications. The promoter activity is deduced from the respective luciferase measurement relative to the unmodified promoter (see also Example 7). BvFT2-control represents the unmodified promoter (SEQ ID NO: 213). In variant BvFT2-50-E038h (SEQ ID NO: 214) the as1-like cis-regulatory element E038h (SEQ ID NO: 7) is inserted at the −50 position. In the variants BvFT2+40-E085 (SEQ ID NO: 215) and BvFT2+40-E086 (SEQ ID NO: 216) the Y-patch core promoter elements E085 (SEQ ID NO: 203) or E086 (SEQ ID NO: 204) are inserted at the +40 position. In the variants BvFT2-50-E038h+40-E085 (SEQ ID NO: 217) and BvFT2-50-E038h+40-E086 (SEQ ID NO: 218) the additional cis-regulatory element and the Y-patch are both present. All modifications displayed result in promoter activation. The combined addition of a CRE and a Y-patch CPE leads to synergistic promoter activation.

FIG. 8A: The upper part of the figure displays a sketch of the Zm-prom2 promoter with positions indicated. B: The graph shows the results from transient testing of the promoter modifications. The promoter activity is deduced from the respective luciferase measurement relative to the unmodified promoter (see Example 8). Zm-prom2 control represents the unmodified promoter. The as1-like CRE E039g (SEQ ID NO: 5) is inserted at different positions (−108, −81, −60 and +86) in relation to an additional TATA-box in position v8-2. The distance between CRE and CPE ranges between 27 bp and 220 bp. In all cases a synergistic enhancement of expression is observed.

FIG. 9A: The upper part of the figure displays a sketch of the ZmCWI3 promoter with positions indicated. B: The graph shows the results from transient testing of the promoter modifications. The promoter activity is deduced from the respective luciferase measurement relative to the unmodified promoter (see Example 9). ZmCWI3 control represents the unmodified promoter (SEQ ID NO: 184). The as1-like CRE E039g (SEQ ID NO: 5) is inserted at different positions (−59 and −51) in relation to the optimized TATA-box in position v3-2 (SEQ ID NOs: 219 und 220). The distance between CRE and CPE ranges between 26 bp and 18 bp. The 18 bp distance between CRE and CPE works optimal to achieve maximal synergistic promoter activation.

FIG. 10A: The upper part of the figure displays a sketch of the Zm-prom7 promoter with positions indicated. B: The graph shows the results from transient testing of the promoter modifications. The promoter activity is deduced from the respective luciferase measurement relative to the unmodified promoter (see Example 10). Zm-prom7 control represents the unmodified promoter. The as1-like CRE E039g (SEQ ID NO: 5) is inserted at different positions (−50, −1 and +8) in relation to an additional TATA-box in position v7. The distance between CRE and CPE ranges between 18 bp and 118 bp. The 18 bp distance between CRE and CPE works optimal to achieve maximal synergistic promoter activation.

FIG. 11A: The upper part of the figure displays a sketch of the Zm-prom8 promoter with positions indicated. B: The graph shows the results from transient testing of the promoter modifications. The promoter activity is deduced from the respective luciferase measurement relative to the unmodified promoter (see Example 11). Zm-prom8 control represents the unmodified promoter. The as1-like CRE E039g (SEQ ID NO: 5) is inserted at different positions (−31 and +9) with respect to an additional TATA-box either generated in position v2 or in position v3-2. The distance between CRE and CPE is 26 bp in both modified promoters possessing the combination Zm-prom8_v2-31-E39g or Zm-prom8_v3-2+9-E39g. Both CRE-CPE combinations lead to synergistic promoter activation. An optimal position for the inserted TATA-box is more important than the position of the CRE.

| Sequence List: | |
|---|---|
| SEQ ID NO: 1 | as1-like element double consensus |
| SEQ ID NO: 2 | double G-box element consensus |
| SEQ ID NO: 3 | Y-patch motif consensus |
| SEQ ID NO: 4 | Y-patch motif example |
| SEQ ID NO: 5 | as1-like E039g |
| SEQ ID NO: 6 | as1-like E038f |
| SEQ ID NO: 7 | as1-like E038h |
| SEQ ID NO: 8 | double G-box E128 |
| SEQ ID NOs: 9 | exemplary cis-regulatory elements |
| to 183 | |
| SEQ ID NO: 184 | ZmCWI3 promoter |
| SEQ ID NO: 185 | ZmCWI3v2 promoter with additional TATA box at |
| | position v2 |
| SEQ ID NO: 186 | ZmCWI3v3-2 promoter with optimized endogenous |
| | TATA-box at position v3-2 |
| SEQ ID NO: 187 | ZmCWI3-50-E039g promoter with CRE (E039g) |
| | inserted at −50 position relative to TSS |
| SEQ ID NO: 188 | ZmCWI3v2-50-E039g promoter with TATA-box at |
| | position v2 and CRE (E039g) inserted at −50 |
| | position relative to TSS |
| SEQ ID NO: 189 | ZmCWI3v3-2-50-E039g promoter with TATA-box |
| | at position v3-2 and CRE (E039g) inserted at −50 |
| | position relative to TSS |
| SEQ ID NO: 190 | BvHPPD1 promoter |
| SEQ ID NO: 191 | BvHPPD1v3 promoter with additional TATA-box at |
| | position v3 |
| SEQ ID NO: 192 | BvHPPD1v4 promoter with additional TATA-box at |
| | position v4 |
| SEQ ID NO: 193 | BvHPPD1v5 promoter with additional TATA-box at |
| | position v5 |
| SEQ ID NO: 194 | BvHPPD1-50-E038f promoter with CRE (E038f) |
| | inserted at −50 position relative to TSS |
| SEQ ID NO: 195 | BvHPPD1v3-50-E038f promoter with additional |
| | TATA-box at position v3 and CRE (E038f) inserted |
| | at −50 position relative to TSS |
| SEQ ID NO: 196 | BvHPPD1v4-50-E038f promoter with additional |
| | TATA-box at position v4 and CRE (E038f) inserted |
| | at −50 position relative to TSS |
| SEQ ID NO: 197 | BvHPPD1v5-50-E038f promoter with additional |
| | TATA-box at position v5 and CRE (E038f) inserted |
| | at −50 position relative to TSS |
| SEQ ID NO: 198 | as1-like E039i |
| SEQ ID NO: 199 | double G-box E133 |
| SEQ ID NO: 200 | TEF-box promoter motif E016 |
| SEQ ID NO: 201 | corn CYP promoter fragment E101c |
| SEQ ID NO: 202 | corn adh1 promoter element E115d |
| SEQ ID NO: 203 | Y-patch E085 |
| SEQ ID NO: 204 | Y-patch E086 |
| SEQ ID NO: 205 | BvHPPD1-50-E133 promoter with CRE (E133) |
| | inserted at −50 position relative to TSS |
| SEQ ID NO: 206 | BvHPPD1v5-50-E133 promoter with additional |
| | TATA-at pobox sition v5 and CRE (E133) inserted |
| | at −50 position relative to TSS |
| SEQ ID NO: 207 | BvHPPD2 promoter |
| SEQ ID NO: 208 | BvHPPD2v5 promoter with additional TATA-box |
| | at position v5 |
| SEQ ID NO: 209 | BvHPPD2-50-E038h promoter with CRE (E038h) |
| | inserted at −50 position relative to TSS |
| SEQ ID NO: 210 | BvHPPD2-50-E128 promoter with CRE (E128) |
| | inserted at −50 position relative to TSS |
| SEQ ID NO: 211 | BvHPPD2v5-50-E038h promoter with additional |
| | TATA-box at position v5 and CRE (E038h) inserted |
| | at −50 position relative to TSS |
| SEQ ID NO: 212 | BvHPPD2v5-50-E128 promoter with additional |
| | TATA-box at position v5 and CRE (E128) |
| | inserted at −50 position relative to TSS |
| SEQ ID NO: 213 | BvFT2 promoter |
| SEQ ID NO: 214 | BvFT2-50-E038h promoter with CRE (E038h) |
| | inserted at −50 position relative to TSS |
| SEQ ID NO: 215 | BvFT2+40-E085 promoter with Y-patch E085 |
| | inserted at +40 position relative to TSS |
| SEQ ID NO: 216 | BvFT2+40-E086 promoter with Y-patch E086 |
| | inserted at +40 position relative to TSS |
| SEQ ID NO: 217 | BvFT2-50-E038h+40-E085 promoter with Y-patch |
| | E085 inserted at +40 position relative to TSS and |
| | CRE (E038h) inserted at −50 position relative to |
| | TSS |
| SEQ ID NO: 218 | BvFT2-50-E038h+40-E086 promoter with Y-patch |
| | E086 inserted at +40 position relative to TSS and |
| | CRE (E038h) inserted at −50 position relative to |
| | TSS |
| SEQ ID NO: 219 | ZmCWI3v3-2-51-E039g promoter with TATA-box |
| | at position v3-2 and CRE (E039g) inserted at −51 |
| | position relative to TSS |
| SEQ ID NO: 220 | ZmCWI3v3-2-59-E039g promoter with TATA-box |
| | at position v3-2 and CRE (E039g) inserted at −59 |
| | position relative to TSS |
| SEQ ID NO: 221 | TEF-box promoter motif consensus sequence |
| SEQ ID NO: 222 | TEF-box promoter motif preferred consensus |
| | sequence |

### DETAILED DESCRIPTION

The present invention relates to several aspects to establish a new technology, which allows to activate the expression of genes by introducing only minor modifications into the promoter sequence. Surprisingly, it was discovered in the context of the present invention, that the combined introduction of a cis-regulating element (CRE) and a core promoter element (CPE) within a certain range of a promoter sequence provides a synergistic enhancement of gene expression. The technology is applicable to a range of target genes and plants.

In one aspect, the present invention relates to a method for increasing the expression level of a nucleic acid molecule of interest in a plant cell, the method comprising
(i) introducing a modification in the nucleic acid sequence of an endogenous promoter controlling the expression of the nucleic acid molecule of interest in a first location so that a cis-regulatory element selected from an as1-like element, a G-box element, a double G-box element, a TEF-box promoter motif, a corn CYP promoter fragment and a corn adh1 promoter element is formed and in a second location so that a core promoter element selected from a TATA box motif, a Y-patch motif, an initiator element and a downstream promoter element is formed, and
(ii) obtaining at least one plant cell showing an increased expression level of the nucleic acid molecule of interest compared to the expression level of the nucleic acid molecule of interest under the control of the unmodified endogenous promoter,
(iii) optionally, culturing the at least one plant cell obtained in step (ii) to obtain a plant showing an increased expression level of the nucleic acid molecule of interest compared to the expression level of the nucleic acid molecule of interest under the control of the unmodified endogenous promoter,
   wherein the first location is located upstream of the second location and the first and the second location are located at a distance of 5 to 225 nucleotides from each other, preferably at a distance of 10 to 160 nucleotides.

It was established in the context of the present invention that by introducing a cis-regulatory element and a core promoter element into a promoter, which is endogenous to a plant cell, the expression the nucleic acid molecule under the control of the endogenous promoter can be significantly increased if the cis-regulatory element and the core promoter elements are introduced within a certain distance from each other.

The first and the second location are located at a distance of a certain number of nucleotides from each other if the specified number of nucleotides is present between the end of the sequence of one of the cis-regulatory element and the core promoter element and the beginning of the sequence of the respective other element once they are introduced.

In a preferred embodiment of the method described above, a second location is identified at a position −300 to −60 nucleotides relative to the start codon of the nucleic acid molecule of interest.

In a preferred embodiment of the method described above, the method comprises the identification of a second location within the endogenous promoter sequence and the first location is positioned upstream thereto, at a distance of 5 to 225 nucleotides, preferably at a distance of 10 to 160 nucleotides, more preferably at a distance of 15 to 60 nucleotides.

In another embodiment of the method described above, the method comprises the identification of a first location within the endogenous promoter sequence and second location is positioned downstream thereto, at a distance of 5 to 225 nucleotides, preferably at a distance of 10 to 160 nucleotides, more preferably at a distance of 15 to 60 nucleotides.

The first or second location can be identified by searching the sequence of the endogenous promotor for a sequence portion, which can be modified to a core promoter element or cis-regulatory element with minimal nucleotide exchanges or which enables the integration of synthetic sequence parts.

In one embodiment of the method according to any of the embodiments described above, at least one of the first and the second location is located downstream of the transcription state site.

An enhancement of expression is also achieved when one or both, the cis-regulatory element and the core promoter element is/are located downstream of the transcription start site for the nucleic acid molecule of interest.

In another embodiment of the method according to any of the embodiments described above, in step (i) less than 30 nucleotides are inserted, deleted and/or substituted at the first and/or the second location, preferably less than 25 nucleotides, preferably less than 20 nucleotides, preferably less than 15 nucleotides.

Advantageously, the modification at the first and second location may only involve the insertion, deletion and/or substitution of a few nucleotides. Thus, it is not necessary to produce transgenic plants in order to provide the desired increased expression levels. The modifications can be introduced by random mutagenesis or by site-specific techniques using tools, which are known to the skilled person as described in further detail below.

In one embodiment of the method according to any of the embodiments described above, the modification in the first and/or second location is introduced by mutagenesis or by site-specific modification techniques using a site-specific nuclease or an active fragment thereof and/or a base editor and/or a prime editor.

In a another embodiment of the method according to any of the embodiments described above, step (i) comprises introducing into the cell a site-specific nuclease or an active fragment thereof, or providing the sequence encoding the same, the site-specific nuclease inducing a single- or double-strand break at a predetermined location, preferably wherein the site-specific nuclease or the active fragment thereof comprises a zinc-finger nuclease, a transcription activator-like effector nuclease, a CRISPR/Cas system, including a CRISPR/Cas9 system, a CRISPR/Cpf1 system, a CRISPR/C2C2 system a CRISPR/CasX system, a CRISPR/CasY system, a CRISPR/Cmr system, a CRISPR/MAD7 system, a CRISPR/CasZ system, an engineered homing endonuclease, a recombinase, a transposase and a meganuclease, and/or any combination, variant, or catalytically active fragment thereof; and optionally when the site-specific nuclease or the active fragment thereof is a CRISPR nuclease: providing at least one guide RNA or at least one guide RNA system, or a nucleic acid encoding the same; and optionally providing at least one repair template nucleic acid sequence.

In a preferred embodiment of the method described in any of the embodiments above, the core promoter element is a TATA box motif having the sequence of CTATAAATA.

The sequence CTATAAATA represents an optimized TATA box motif, which is able to provide a significant increase in expression levels in combination with a cis-regulatory element.

In another preferred embodiment of the method described in any of the embodiments above, the core promoter element is a Y-patch motif having a sequence according to the sequence of SEQ ID NO: 203 or 204.

In one embodiment of the method according to any of the embodiments described above, the cis-regulatory element is selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or has a sequence being 95%, 96%, 97%, 98% or 99% identical to any of the sequences of SEQ ID NOs: 5 to 8 or 198 to 202.

In a preferred embodiment of the method according to any of the embodiments described above, the first and the second location are located at a distance of 15 to 60 nucleotides from each other.

As demonstrated in the examples, if the cis-regulatory element and the core promoter element are located at a distance of 15 to 60 nucleotides from each other, an increase in expression in a range of 30 to 417 fold is observed.

In another embodiment of the method according to any of the embodiments described above, the expression level of the nucleic acid of interest controlled by the modified endogenous promoter is increased at least 20-fold, increased at least 50-fold, increased at least 100-fold, increased at least 150-fold, increased at least 200-fold, increased at least 250-fold, increased at least 300-fold, increased at least 350-fold, increased at least 400-fold in comparison to the expression level of the nucleic acid molecule of interest under the control of the unmodified endogenous promoter.

In one embodiment of the method described above, an increased expression in a range from 2 fold to 500 fold is obtained when the cis-regulatory element and the core promoter element are located at a distance of 5 to 225 nucleotides, preferably 10 to 160 nucleotides, more preferably 15 to 60 nucleotides from each other.

In another aspect, the present invention relates to a promoter, which is endogenous to a plant cell and which has been modified to provide an increased expression level of a nucleic acid molecule of interest in a plant cell, wherein the promoter has been modified to comprise
(a) a cis-regulatory element, which is heterologous to the promoter, selected from an as1-like element, a G-box element, a double G-box element, a TEF-box promoter motif, a corn CYP promoter fragment and a corn adh1 promoter element, and
(b) a TATA box motif having the sequence of CTATAAATA and being heterologous to the promoter,
wherein the cis-regulatory element is located upstream of the TATA box motif and the cis-regulatory element and the TATA box motif are positioned at a distance of 5 to 225 nucleotides from each other, preferably positioned at a distance of 10 to 160 nucleotides from each other, and preferably wherein the expression level provided by the endogenous modified promoter is increased synergistically with respect to the endogenous promoter comprising only said cis-regulatory element or said TATA box motif sequence.

The two elements (i.e. the cis-regulatory element and the TATA box motif are located at a distance of a certain number of nucleotides from each other when the number of nucleotides is present between the end of the sequence of one element and the beginning of the sequence of the other element.

Preferably, the TATA box motif is located at a position −300 to −60 nucleotides relative to the start codon of a nucleic acid sequence expressed under the control of the promoter, i.e. 300 to 60 nucleotides upstream of the end of the promoter sequence.

A promoter, which is endogenous to a plant cell can be modified to increase the expression level of the nucleic acid molecule, which is expressed under the control of the promoter. Thus, certain positive traits of a plant can be enhanced.

In one embodiment of the modified promoter described above, at least one of the cis-regulatory element and the TATA box motif are located downstream of the transcription start site.

In another embodiment of the modified promoter according to any of the embodiments described above, the modified promoter provides an increased expression level of a nucleic acid molecule of interest compared to the expression level of a nucleic acid molecule of interest under the control of the unmodified endogenous promoter.

In a preferred embodiment of the modified promoter according to any of the embodiments described above, the cis-regulatory element and the TATA box motif are located at a distance of 15 to 60 nucleotides from each other.

In one embodiment of the modified promoter according to any of the embodiments described above, the expression level of an nucleic acid of interest controlled by the modified endogenous promoter is increased at least 20-fold, increased at least 50-fold, increased at least 100-fold, increased at least 150-fold, increased at least 200-fold, increased at least 250-fold, increased at least 300-fold, increased at least 350-fold, increased at least 400-fold in comparison to the expression level of the nucleic acid molecule of interest under the control of the unmodified endogenous promoter.

In another embodiment of the modified promoter according to any of the embodiments described above, the cis-regulatory element is selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or has a sequence being 95%, 96%, 97%, 98% or 99% identical to any of the sequences of SEQ ID NOs: 5 to 8 or 198 to 202.

In another aspect, the present invention also relates to a nucleic acid molecule comprising or consisting of a promoter sequence, which is endogenous to a plant cell and which has been modified to comprise
(a) a cis-regulatory element selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or having a sequence being 95%, 96%, 97%, 98% or 99% identical to any of the sequences of SEQ ID NOs: 5 to 8 or 198 to 202, and
(b) a TATA box motif having the sequence of CTATAAATA, located at a position −300 to −60 nucleotides relative to the start codon,
wherein (a) and (b) are located at a distance of 15 to 60 nucleotides to each other,
and preferably wherein the expression level provided by the modified endogenous promoter is increased at least 20-fold with respect to a promoter comprising no modification and wherein the expression level provided by the promoter is increased synergistically with respect to an endogenous promoter comprising only said cis-regulatory element or said TATA box motif.

Preferably, the cis-regulatory element and the TATA box motif are heterologous to the promoter sequence.

The TATA box motif is located at a position −300 to −60 nucleotides relative to the start codon of a nucleic acid sequence expressed under the control of the promoter meaning that it is located 60 to 300 nucleotides upstream of the end of the promoter sequence.

The two elements (i.e. the cis-regulatory element and the TATA box motif are located at a distance of a certain number of nucleotides from each other when the number of nucleotides is present between the end of the sequence of one element and the beginning of the sequence of the other element.

In one embodiment of the nucleic acid molecule described above, at least one of the cis-regulatory element and the core promoter element are located downstream of the transcription start site.

In a further aspect, the present invention also relates to a plant cell or a plant obtained or obtainable by a method according to any of the embodiments described above.

In another aspect, the present invention relates to the use of a nucleic acid molecule according to any of the embodiments described above, or the use of a modified promoter according to any of the embodiments described above for increasing the expression level of a nucleic acid molecule of interest in a plant cell, preferably in a method according to any of the embodiments described above.

In one embodiment according to the various aspects and embodiments of the present invention described herein, the cis-regulatory element may also originate from a virus or phage, the virus or phage being selected from the group consisting of Sugarcane bacilliform virus (NCBI accession number: MK632870.1), Sugarcane bacilliform virus (KY031904.1), Sugarcane bacilliform virus (JN377537.1), Sugarcane bacilliform IM virus (AJ277091.1), Banana streak Peru virus (MN187554.1), Grapevine vein clearing virus (MH319694.1), Grapevine vein clearing virus (MH319693.1), Sugarcane bacilliform virus (KT186240.1), Grapevine vein clearing virus (KX610317.1), Grapevine vein clearing virus (KX610316.1), Sugarcane bacilliform virus (KJ624754.1), Grapevine vein clearing virus (KT907478.1), Grapevine vein clearing virus (KJ725346.1), Sugarcane bacilliform virus (JN377535.1), Grapevine vein clearing virus (MN716781.1), Grapevine vein clearing virus (MN716771.1), Grapevine vein clearing virus (JF301669.2), Sugarcane bacilliform virus (FJ824814.1), Sugarcane bacilliform Guadeloupe A virus (NC_038382.1), Banana streak virus (DQ674317.1), Canna yellow mottle virus (KX255726.1), Canna yellow mottle virus (KX255725.1), Canna yellow mottle associated virus (KX066020.1), Banana streak virus (KT895259.1), Sugarcane bacilliform virus (KM214357.1), Sugarcane bacilliform virus (JN377534.1), Banana streak UA virus (HQ593107.1), Sugarcane bacilliform MO virus (M89923.1), Banana streak virus (KT895258.1), Banana streak IM virus (KJ013508.1), Myoviridae sp. (BK023605.1), My-oviridae sp. (BK044012.1), Bacteriophage sp. (BK021256.1), Lactobacillus phage ATCC 8014-B2 (NC_047739.1), Banana streak IM virus (HQ593112.1), Banana streak UI virus (HQ593108.1), Banana streak IM virus (HQ659760.1), Banana streak IM virus (HQ677570.1), Banana streak virus (FJ527425.1), Banana streak virus (FJ527424.1), Banana streak virus (FJ527423.1), Banana streak IM virus (AB252638.1), Erwinia phage pEa_SNUABM_36 (MZ443789.1), Erwinia phage pEa_SNUABM_35 (MZ443788.1), Cau-dovirales sp. (BK054845.1), Marine virus AFVG_25M72 (MN693380.1), Marine virus AFVG_25M71 (MN693300.1), Pelagibacter phage HTVC106P (MN698244.1), Echovirus E6 (KC962392.1), Pseudomonas phage PaBG (NC_022096.1), Caudovirales sp. (BK026925.1), Myoviridae sp. (BK029906.1), Siphoviridae sp. (BK048414.1), Myoviridae sp. (BK056877.1), Myoviridae sp. (BK045003.1), Myoviridae sp. (BK050010.1), Barley yellow dwarf virus GPV (EF174415.1), Wenzhou tombus-like virus 12 (KX883262.1), Siphoviridae sp. (BK048144.1), Siphoviridae sp. (BK029857.1), Siphoviridae sp. (BK030436.1), Myoviridae sp. (BK032287.1), Siphoviridae sp. (BK038535.1), Sugarcane bacilliform virus (JN377533.1), Sugarcane bacilliform virus (MW302331.1), Marine virus AFVG_25M508 (MN693521.1), Vibrio phage 6E35.1a (MW824377.1), Myoviridae sp. (BK057854.1), Podoviridae sp. (BK030164.1), Human immunodeficiency virus 1 (MZ346930.1), Human immunodeficiency virus 1 (MZ346928.1), Human immunodeficiency virus 1 (MZ346920.1), Human immunodeficiency virus 1 (MZ346919.1), Human immuno-deficiency virus 1 (MZ346917.1), Human immunodeficiency virus 1 (MZ346933.1), Human immunodeficiency virus 1 (MZ346932.1), Human immunodeficiency virus 1 (MZ346931.1), Human immunodeficiency virus 1 (MZ346929.1), Human immunodeficiency virus 1 (MZ346927.1), Human immunodeficiency virus 1 (MZ346926.1), Human immunodeficiency virus 1 (MZ346925.1), Human immunodeficiency virus 1 (MZ346924.1), Human immuno-deficiency virus 1 (MZ346923.1), Human immunodeficiency virus 1 (MZ346921.1), Human immunodeficiency virus 1 (MZ346916.1), Human immunodeficiency virus 1 (JX447027.1), Human immunodeficiency virus 1 (JX447026.1), Human immunodeficiency virus 1 (JX447025.1), Human immunodeficiency virus 1 (JX447024.1), Human immunodeficiency virus 1 (JX447023.1), Human immunodeficiency virus 1 (JX447022.1), Human immunode-ficiency virus 1 (JX447021.1), Human immunodeficiency virus 1 (JX447020.1), Human immunodeficiency virus 1 (JX447019.1), Human immunodeficiency virus 1 (JX447018.1), Siphoviridae sp. (BK037671.1), Siphoviridae sp. (BK052943.1), Bacillus phage Tomato (MT584805.1), Fowlpox virus (MH734528.1), Fowlpox virus (MH719203.1), Fowlpox virus (MH709125.1), Fowlpox virus (MH709124.1), Fowlpox virus (MF766432.1), Fowlpox virus (MF766431.1), Fowlpox virus (MF766430.1), Fowlpox virus (OK558609.1), Fowlpox virus (OK558608.1), Fowlpox virus (MW142017.1), Fowlpox virus (KX196452.1), Fowlpox virus isolate HP-438/Munich (AJ581527.1) and Fowlpox virus (AF198100.1).

In one aspect the present invention relates to a nucleic acid molecule comprising or consisting of a promoter active in a plant cell, comprising
(a) a cis-regulatory element, and
(b) a core promoter element,
wherein the cis-regulatory element is located upstream of the core promoter element and the cis-regulatory element, and the core promoter element are located at a distance of 5 to 225 nucleotides from each other, preferably 10 to 160 nucleotides, particularly preferably 15 to 60 nucleotides, and wherein the expression level provided by the promoter is increased synergistically with respect to a promoter comprising only one of the cis-regulatory element and the core promoter element. The two elements being located at a distance of 5 to 225 nucleotides etc. from each other means that there are 5 to 225 nucleotides in between the end of the sequence of one element and the start of the sequence of the other element.

Preferably, the nucleic acid molecule of the present invention is an artificial or synthetic molecule, which does not occur in nature. As demonstrated in the examples, when a cis-regulatory element is present upstream of a core promoter element in a promoter and the two elements are not separated by more than 225 nucleotides, preferably no more than 160 nucleotides, particularly preferably no more than 60 nucleotides a synergistic enhancement of expression of the nucleic acid sequence under the control of the promoter is observed. Synergism in this context is defined as more than the sum of the enhancement achieved by only the cis-regulatory element or only the core promoter element.

Cis-regulatory elements represent binding sites for transcription factors and their presence within a certain range of the promoter can enhance the expression of the nucleic acid sequence expressed under the control of the promoter. Examples of cis-regulatory elements identified by specific sequences or by conserved motifs are given below.

Core promoter elements play an essential role in transcription initiation as the first step of gene expression. Core promoter elements can be identified by certain conserved motifs, which define a core promoter consensus sequence. The actual sequence of the respective motifs in a given promoter is characteristic for the activity of the promoter and thus for the expression level of the expression product under its control. Certain “ideal” core promoter element sequences have an expression enhancing effect, while the expression decreases gradually if the sequence deviates from the ideal sequence. A nucleic acid of the present invention may comprise more than one core promoter element. In particular, it is possible that a native core promoter element is supplemented with another core promoter element at a different position or with an optimized sequence to achieve synergistic enhancement together with the cis-regulatory element. Examples of core promoter elements identified by specific sequences or by conserved motifs are given below.

In a preferred embodiment, the nucleic acid molecule comprises a CPE as defined herein in addition to an endogenous CPE. In another preferred embodiment, the nucleic acid molecule comprises an optimized CPE as defined herein, which was generated by modification of an endogenous CPE.

The application is not limited to certain promoters or nucleic acid sequences to be expressed or combinations of both. In one embodiment the nucleic acid sequence to be expressed is endogenous to the plant cell that it is expressed in. In this case, the promoter may be the promoter that natively controls the expression of the nucleic acid sequence but it is also possible that an endogenous nucleic acid sequence is expressed under the control of a heterologous promoter, which does not natively control its expression. Alternatively, the nucleic acid sequence is exogenous to the plant cell that it is expressed in. In this case, the promoter may also be exogenous to the plant but it may be the promoter that the nucleic acid sequence is controlled by in its native cellular environment. On the other hand, the promoter may also be exogenous to the plant cell and at the same time be heterologous to the nucleic acid sequence.

Advantageously, the enhancement can be applied to the expression of a trait gene, i.e. a gene that provides desirable agronomic traits such as resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, resistance or tolerance to 2,4-D, protoporphyrinogen oxidase (PPO) inhibitors, ALS inhibitors, and Dicamba, a nucleic acid molecule encoding resistance or tolerance to biotic stress, including a viral resistance gene, a fungal resistance gene, a bacterial resistance gene, an insect resistance gene, or a nucleic acid molecule encoding a yield related trait, including lodging resistance, flowering time, shattering resistance, seed color, endosperm composition, or nutritional content.

The trait gene can be an endogenous gene to the plant cell, but it can also be a transgene, which was introduced into the plant cell by biotechnological means, optionally together with the promoter controlling its expression.

In a preferred embodiment of the nucleic acid molecule described above, the promoter is a promoter derived from Zea mays (Zm) or from Beta vulgaris (Bv). Particularly preferred is a promoter selected from the group consisting of ZmCWI3, BvHPPD1, BvHPPD2 and BvFT2.

In one embodiment of the nucleic acid molecule described above, the core promoter element is located at a position −300 to −60 nucleotides relative to the start codon.

The core promoter element is preferably located within a distance of 60 to 300 nucleotides upstream of the start codon of the nucleic acid sequence, the expression of which is controlled by the promoter. Accordingly, the core promoter element as well as the upstream cis-regulatory element may be downstream of the transcription start site, e.g., within the 5′-untranslated region. In the nucleic acid molecule of the invention, the cis-regulatory element is preferably located within a distance of −130 to +160 relative to the transcription start site.

In one embodiment of the nucleic acid molecule according to any of the embodiments described above, at least one of the cis-regulatory elements and the core promoter element is located downstream of the transcription start site. In another embodiment of the nucleic acid molecule described above, both the cis-regulatory element and the core promoter element are located downstream of the transcription start site.

In one embodiment of the nucleic acid molecule according to any of the embodiments described above, the cis-regulatory element is selected from an as1-like element, a G-box element, a double G-box element, a TEF-box promoter motif, a corn CYP promoter fragment and a corn adh1 promoter element.

In one embodiment of the nucleic acid molecule according to any of the embodiments described above, the cis-regulatory element comprises a sequence motif selected from TKACG and CACGTG, wherein K stand for G or T. Preferably, K stands for G.

In another embodiment of the nucleic acid molecule according to any of the embodiments described above, the cis-regulatory element comprises two TKACG or two CACGTG motifs, wherein K stands for G or T. Preferably, K stands for G. In this embodiment, the two TKACG or the two CACGTG are either in tandem or are separated by 1, 1, 2, 3, 4, 5, 6, 7, 8, 9, 10 or up to 15, up to 20, up to 25, up to 30, up to 35, up to 40, up to 45 or up to 50 arbitrary nucleotide(s).

In one embodiment of the nucleic acid molecule according to any of the embodiments described above, the cis-regulatory element comprises a sequence selected from the sequences of SEQ ID NO: 1 and 2, wherein N stands for 0, 1, 2, 3, 4, 5, 6, 7, 8, 9, 10 or up to 15, up to 20, up to 25, up to 30, up to 35, up to 40, up to 45 or up to 50 arbitrary nucleotide(s).

In another embodiment of the nucleic acid molecule according to any of the embodiments above, the cis-regulatory element comprises a sequence selected from any of SEQ ID NOs: 5 to 8 and 198 to 202, or a sequence being 95%, 96%, 98% or 99% identical to any of these sequences.

In yet another embodiment of the nucleic acid molecule according to any of the embodiments described above, the cis-regulatory element comprises a motif selected from AAAAAGG, GCCGCA, TTCTAGAA, GCACGTGB, TAATNATTA, ACACGTGT, AGATTCT, GCGGCCG, TAATAATT, CGGTAAA, VTGACGT, CCGTTA, CCTCGT, AAAGBV, GGSCCCAC, CTTGACYR, CRCCGACA, AGATTTT, TGTCGGTG, GGNCCCAC, NNTGTCGGN, ATAATTAT, NAAAAGBGN, ATGTCGGC, NVGCCGNC, AGATATTT, TCCGGA, GCCGTC, AATNATTA, GAATAWT, TTACGTGT, VAAAAAGTN, CGTTGACY, RCCGACA, TAATNATT, AATTAAAT, AAWTAWTT, TTAATTAA, TCAATCA, CTGCATGCA, AATGATTG, MACCGMCW, ACCTACG, CACCGACA, AGATACG, AACCTTAA, GAAGCTTC, NBGTCGGYN, NNCACGTGN, TGTCGA, NNNVCGCGT, AGATATG, GCCGCC, AACGTG, GTTAACG, RYCGACAT, WGNTGTAG, ATGTCGGY, NAAAGBN, AAAATAAT, VGAATCTN, CGCCGCCS, CCACGTGG, GGATCC, GTTAGTTR, AGTNNACT, GCCGAC, CGTAC, NTAATTAAN, ACACGTGG, NAAAGB, ACACTA, CCACTTGN, AAAAAGTG, GGTWGTTR, NVGCCGCCN, CATGTG, CAGCT, NAAAGB, RCCGACCA, GCCGGC, AAAGCN, TCACCA, TGACGTG, GKTKGTTR, ACCGAC, RGATATCY, ACCGACA, CGTGTAG, CGGTAAT, AAGATACG, TTACGTAA, SCGCCGCC, CCGCCGACA, NNNAAAG, AAATATCT, CACGCG, CCAATTATT, GCACGTGC, GGGCCCAC, BCAATNATN, GCGCCGCC, NCCGACANV, AATATATT, GCCGACAT, GCCGACAAV, CAATWATT, AATWATTG, AAATATTT, VCCGACAN, AGATACGS, TGTCGGAA, TTGCGTGT, TGTCGG, CATCATC, TGCCGACAB, RTTAGGT, GGGACCAC, GAATAT, GKTAGGT, CACGAG, TGACGTCA, AATTAATT, GAATATTC, AAAGRBB, AGTNNNACT, GCAGACATN, CACGTG, CCGCGTNNN, CAATSATT, AGATHCGV, AATNATAA, VGCGCCANN, RCCGWCCW, NVGCCGBVN, CACGTGVBN, AGATCT, TTGCGTG, VGCCGCCV, VTGACGTAN, TGACGTM, NACGTGGV and BCACGTGVN or a sequence selected from any of SEQ ID NOs: 9 to 183.

In one embodiment of the nucleic acid molecule according to any of the embodiments described above, the core promoter element is selected from a TATA box motif, a Y-patch motif, an initiator element and a downstream promoter element.

The TATA box consensus sequences is CTATAWAWA, wherein W stand for A or T. An ideal TATA box motif is represented by CTATAAATA. A nucleic acid molecule of the present invention can contain more than one TATA box motifs. In particular, the promoter sequence may comprise a native TATA box motif and an additional TATA box motif at a suitable position to provide the synergistic enhancement of expression.

In one embodiment of the nucleic acid molecule according to any of the embodiments described above, the core promoter element is a TATA box motif comprising a CTATAWAWA motif, wherein W stand for A or T, preferably a CTATAAATA motif.

In a preferred embodiment of the nucleic acid molecule according to any of the embodiments described above, the cis-regulatory element comprises a sequence motif selected from TKACG and CACGTG, wherein K stand for G or T, preferably K stands for G, and the core promoter element is a TATA box motif comprising a CTATAWAWA motif, wherein W stand for A or T, preferably a CTATAAATA motif.

In another preferred embodiment of the nucleic acid molecule according to any of the embodiments described above, the cis-regulatory element comprises two TKACG or two CACGTG motifs, wherein K stands for G or T, preferably K stands for G, and the core promoter element is a TATA box motif comprising a CTATAWAWA motif, wherein W stand for A or T, preferably a CTATAAATA motif. In this embodiment, the two TKACG or the two CACGTG are either in tandem or are separated by 1, 1, 2, 3, 4, 5, 6, 7, 8, 9, 10 or up to 15, up to 20, up to 25, up to 30, up to 35, up to 40, up to 45 or up to 50 arbitrary nucleotide(s).

In one preferred embodiment of the nucleic acid molecule according to any of the embodiments described above, the cis-regulatory element comprises a sequence selected from the sequences of SEQ ID NO: 1 and 2, wherein N stands for 0, 1, 2, 3, 4, 5, 6, 7, 8, 9, 10 or up to 15, up to 20, up to 25, up to 30, up to 35, up to 40, up to 45 or up to 50 arbitrary nucleotide(s) and the core promoter element is a TATA box motif comprising a CTATAWAWA motif, wherein W stand for A or T, preferably a CTATAAATA motif.

In another preferred embodiment of the nucleic acid molecule according to any of the embodiments described above, the cis-regulatory element comprises a sequence selected from any of SEQ ID NOs: 5 to 8, or a sequence being 95%, 96%, 98% or 99% identical to any of these sequences and the core promoter element is a TATA box motif comprising a CTATAWAWA motif, wherein W stand for A or T, preferably a CTATAAATA motif.

In one embodiment of the nucleic acid molecule according to any of the embodiments described above, the nucleic acid molecule comprises a sequence according to any of SEQ ID NOs: 189, 195, 196, 197, 206, 211, 212, 217, 218, 219 and 220 or a sequence being 85%, 90%, 95%, 96%, 98% or 99% identical to any of these sequences.

In another embodiment of the nucleic acid molecule according to any of the embodiments described above, the core promoter element is a Y-patch motif and has a sequence according to SEQ ID NO: 3, wherein Y stands for C or T, preferably a sequence according to SEQ ID NO: 4.

In another embodiment of the nucleic acid molecule according to any of the embodiments described above, the core promoter element has a sequence selected from the sequences of SEQ ID NOs: 203 and 204.

In one embodiment of the nucleic acid molecule according to any of the embodiments above, the cis-regulatory element has a sequence selected from the sequences of SEQ ID NOs: 5, 6, 7, 8, 198, 199, 200, 201 and 202 or a sequence being 95%, 96%, 97%, 98% or 99% identical to any of these sequences and the core promoter element is a TATA box motif comprising a CTATAWAWA motif, wherein W stand for A or T, preferably a CTATAAATA motif.

In another embodiment of the nucleic acid molecule according to any of the embodiments above, the cis-regulatory element has a sequence selected from the sequences of SEQ ID NOs: 5, 6, 7, 8, 198, 199, 200, 201 and 202, preferably SEQ ID NO: 7 or a sequence being 95%, 96%, 97%, 98% or 99% identical to any of these sequences and the core promoter element has a sequence of SEQ ID NO: 203 or 204. In another aspect, the present invention relates to a method for increasing the expression level of a nucleic acid molecule of interest in a plant cell, the method comprising
(i) introducing a modification in the nucleic acid sequence of the original promoter controlling the expression of the nucleic acid molecule of interest in a first location so that a cis-regulatory element is formed and in a second location so that a core promoter element is formed, or
   introducing a nucleic acid molecule as defined in any of the embodiments described above replacing the original promoter, and
(ii) obtaining at least one plant cell showing an increased expression level of the nucleic acid molecule of interest compared to the expression level of the nucleic acid molecule of interest under the control of the unmodified original promoter, and
(iii) optionally, culturing the at least one plant cell obtained in step (ii) to obtain a plant showing an increased expression level of the nucleic acid molecule of interest compared to the expression level of the nucleic acid molecule of interest under the control of the unmodified original promoter,
   wherein the first location is located upstream of the second location and the first and the second location are located at a distance of 5 to 225 nucleotides from each other, preferably 10 to 160 nucleotides, particularly preferably 15 to 60 nucleotides.

By introducing a promoter sequence comprising a combination of a cis-regulatory element and a core promoter element as specified above, the expression level of a nucleic acid molecule of interest can be significantly, and even synergistically, increased. Using the method specified above, it is also possible to increase the expression level of a nucleic acid molecule of interest by only minimal modification to a promoter. The introduction of the modifications at the first and second location may only require the replacement, addition and/or replacement of a few nucleotides. Such modifications can be achieved by various techniques including genome editing (GE) approaches as explained in further detail below.

The original promoter controlling the expression of the nucleic acid molecule of interest before the modification is introduced in step i) may contain a motif, which differs in one or more positions from a consensus sequence of a cis-regulatory element and/or a core promoter element or an ideal motif as disclosed herein. For an increase in promoter activity, resulting in higher expression levels, the sequence of the motif can be altered in a way that it becomes more similar to the consensus sequence or the ideal motif.

In one embodiment of the method described above, the second location is located at a position −300 to −60 nucleotides relative to the start codon of the nucleic acid molecule of interest.

In a preferred embodiment of the method according to any of the embodiments described above, a second location is identified at a position −300 to −60 nucleotides relative to the start codon of the nucleic acid of interest and the first location is determined at an optimal distance upstream of the second location.

Using this two-step approach, the highest enhancement of expression can be achieved.

The core promoter element is preferably located within a distance of 60 to 300 nucleotides upstream of the start codon of the nucleic acid sequence, the expression of which is controlled by the promoter. Accordingly, the core promoter element as well as the upstream cis-regulatory element may be downstream of the transcription start site, e.g., within the 5′-untranslated region.

In one embodiment of the method according to any of the embodiments described above at least one of the first and the second location is located downstream of the transcription start site. In another embodiment of the nucleic acid described above, both the first and the second location are located downstream of the transcription start site.

Preferably, only a few nucleotides are inserted, deleted or substituted in the original promoter sequence to introduce the modifications at the first and second location. Introducing only such minimal modification may allow for a plant carrying the promoter to avoid regulations or restrictions pertaining to transgenic modifications.

In a preferred embodiment of the method according to any of the embodiments above, in step (i) less than 30 nucleotides are inserted, deleted and/or substituted at the first and/or the second location, preferably less than 25 nucleotides, preferably less than 20 nucleotides, preferably less than 15 nucleotides.

In a preferred embodiment of the method described above, the original promoter is a promoter derived from Zea mays (Zm) or from Beta vulgaris (Bv). Particularly preferred is a promoter selected from the group consisting of ZmCWI3, BvHPPD1, BvHPPD2 and BvFT2.

In one embodiment of the method according to any of the embodiments described above, the cis-regulatory element is selected from an as1-like element, a G-box element, a double G-box element, a TEF-box promoter motif, a corn CYP promoter fragment and a corn adh1 promoter element.

In one embodiment of method according to any of the embodiments described above, the cis-regulatory element comprises a sequence motif selected from TKACG and CACGTG, wherein K stand for G or T. Preferably K stands for G.

In another embodiment of the method according to any of the embodiments described above, the cis-regulatory element comprises two TKACG or two CACGTG motifs, wherein K stands for G or T. Preferably K stands for G. In this embodiment, the two TKACG or the two CACGTG are either in tandem or are separated by 1, 1, 2, 3, 4, 5, 6, 7, 8, 9, 10 or up to 15, up to 20, up to 25, up to 30, up to 35, up to 40, up to 45 or up to 50 arbitrary nucleotide(s).

In one embodiment of the method according to any of the embodiments described above, the cis-regulatory element comprises a sequence selected from the sequences of SEQ ID NO: 1 and 2, wherein N stands for 0, 1, 2, 3, 4, 5, 6, 7, 8, 9, 10 or up to 15, up to 20, up to 25, up to 30, up to 35, up to 40, up to 45 or up to 50 arbitrary nucleotide(s).

In another embodiment of the method according to any of the embodiments described above, the cis-regulatory element comprises a sequence selected from any of SEQ ID NOs: 5 to 8 and 198 to 202, or a sequence being 95%, 96%, 98% or 99% identical to any of these sequences.

In yet another embodiment of the method according to any of the embodiments described above, the cis-regulatory element comprises a motif selected from AAAAAGG, GCCGCA, TTCTAGAA, GCACGTGB, TAATNATTA, ACACGTGT, AGATTCT, GCGGCCG, TAATAATT, CGGTAAA, VTGACGT, CCGTTA, CCTCGT, AAAGBV, GGSCCCAC, CTTGACYR, CRCCGACA, AGATTTT, TGTCGGTG, GGNCCCAC, NNTGTCGGN, ATAATTAT, NAAAAGBGN, ATGTCGGC, NVGCCGNC, AGATATTT, TCCGGA, GCCGTC, AATNATTA, GAATAWT, TTACGTGT, VAAAAAGTN, CGTTGACY, RCCGACA, TAATNATT, AATTAAAT, AAWTAWTT, TTAATTAA, TCAATCA, CTGCATGCA, AATGATTG, MACCGMCW, ACCTACG, CACCGACA, AGATACG, AACCTTAA, GAAGCTTC, NBGTCGGYN, NNCACGTGN, TGTCGA, NNNVCGCGT, AGATATG, GCCGCC, AACGTG, GTTAACG, RYCGACAT, WGNTGTAG, ATGTCGGY, NAAAGBN, AAAATAAT, VGAATCTN, CGCCGCCS, CCACGTGG, GGATCC, GTTAGTTR, AGTNNACT, GCCGAC, CGTAC, NTAATTAAN, ACACGTGG, NAAAGB, ACACTA, CCACTTGN, AAAAAGTG, GGTWGTTR, NVGCCGCCN, CATGTG, CAGCT, NAAAGB, RCCGACCA, GCCGGC, AAAGCN, TCACCA, TGACGTG, GKTKGTTR, ACCGAC, RGATATCY, ACCGACA, CGTGTAG, CGGTAAT, AAGATACG, TTACGTAA, SCGCCGCC, CCGCCGACA, NNNAAAG, AAATATCT, CACGCG, CCAATTATT, GCACGTGC, GGGCCCAC, BCAATNATN, GCGCCGCC, NCCGACANV, AATATATT, GCCGACAT, GCCGACAAV, CAATWATT, AATWATTG, AAATATTT, VCCGACAN, AGATACGS, TGTCGGAA, TTGCGTGT, TGTCGG, CATCATC, TGCCGACAB, RTTAGGT, GGGACCAC, GAATAT, GKTAGGT, CACGAG, TGACGTCA, AATTAATT, GAATATTC, AAAGRBB, AGTNNNACT, GCAGACATN, CACGTG, CCGCGTNNN, CAATSATT, AGATHCGV, AATNATAA, VGCGCCANN, RCCGWCCW, NVGCCGBVN, CACGTGVBN, AGATCT, TTGCGTG, VGCCGCCV, VTGACGTAN, TGACGTM, NACGTGGV and BCACGTGVN or a sequence selected from any of SEQ ID NOs: 9 to 183.

In one embodiment of the method according to any of the embodiments described above, the core promoter element is selected from a TATA box motif, a Y-patch motif, an initiator element and a downstream promoter element.

The TATA box consensus sequences is CTATAWAWA, wherein W stand for A or T. An ideal TATA box motif is represented by CTATAAATA. A promoter obtained by the method of the present invention can contain more than one TATA box motifs. In particular, the promoter sequence may comprise a native TATA box motif and an additional TATA box motif at a suitable position to provide the synergistic enhancement of expression.

In one embodiment of the method according to any of the embodiments described above, the core promoter element is a TATA box motif comprising a CTATAWAWA motif, wherein W stand for A or T, preferably a CTATAAATA motif.

In a preferred embodiment of the method according to any of the embodiments described above, the cis-regulatory element comprises a sequence motif selected from TKACG and CACGTG, wherein K stand for G or T, preferably K stands for G, and the core promoter element is a TATA box motif comprising a CTATAWAWA motif, wherein W stand for A or T, preferably a CTATAAATA motif.

In another preferred embodiment of the method according to any of the embodiments described above, the cis-regulatory element comprises two TKACG or two CACGTG motifs, wherein K stands for G or T, preferably K stands for G, and the core promoter element is a TATA box motif comprising a CTATAWAWA motif, wherein W stand for A or T, preferably a CTATAAATA motif. In this embodiment, the two TKACG or the two CACGTG are either in tandem or are separated by 1, 1, 2, 3, 4, 5, 6, 7, 8, 9, 10 or up to 15, up to 20, up to 25, up to 30, up to 35, up to 40, up to 45 or up to 50 arbitrary nucleotide(s).

In one preferred embodiment of the method according to any of the embodiments described above, the cis-regulatory element comprises a sequence selected from the sequences of SEQ ID NO: 1 and 2, wherein N stands for 0, 1, 2, 3, 4, 5, 6, 7, 8, 9, 10 or up to 15, up to 20, up to 25, up to 30, up to 35, up to 40, up to 45 or up to 50 arbitrary nucleotide(s) and the core promoter element is a TATA box motif comprising a CTATAWAWA motif, wherein W stand for A or T, preferably a CTATAAATA motif.

In another preferred embodiment of the method according to any of the embodiments described above, the cis-regulatory element comprises a sequence selected from any of SEQ ID NOs: 5 to 8, or a sequence being 95%, 96%, 98% or 99% identical to any of these sequences and the core promoter element is a TATA box motif comprising a CTATAWAWA motif, wherein W stand for A or T, preferably a CTATAAATA motif.

In one embodiment of the method according to any of the embodiments above, the nucleic acid molecule replacing the original promoter comprises a sequence according to any of SEQ ID NOs: 189, 195, 196, 197, 206, 211, 212, 217, 218, 219 and 220 or a sequence being 85%, 90%, 95%, 96%, 98% or 99% identical to any of these sequences.

In another embodiment of the method according to any of the embodiments above, the core promoter element is a Y-patch motif and has a sequence according to SEQ ID NO: 3, wherein Y stands for C or T, preferably a sequence according to SEQ ID NO: 4.

In another embodiment of the method according to any of the embodiments described above, the core promoter element has a sequence selected from the sequences of SEQ ID NO: 203 and 204.

In one embodiment of the method according to any of the embodiments above, the cis-regulatory element has a sequence selected from the sequences of SEQ ID NOs: 5, 6, 7, 8, 198, 199, 200, 201 and 202 or a sequence being 95%, 96%, 97%, 98% or 99% identical to any of these sequences and the core promoter element is a TATA box motif comprising a CTATAWAWA motif, wherein W stand for A or T, preferably a CTATAAATA motif.

In another embodiment of the method according to any of the embodiments above, the cis-regulatory element has a sequence selected from the sequences of SEQ ID NOs: 5, 6, 7, 8, 198, 199, 200, 201 and 202, preferably SEQ ID NO: 7 or a sequence being 95%, 96%, 97%, 98% or 99% identical to any of these sequences and the core promoter element has a sequence of SEQ ID NO: 203 or 204. In one embodiment of the method according to any of the embodiments described above, the modification in the first and/or second location is introduced by mutagenesis or by site-specific modification techniques using a site-specific nuclease or an active fragment thereof and/or a base editor and/or a prime editor.

Mutagenesis techniques can be based on chemical induction (e.g., EMS (ethyl methanesulfonate) or ENU (N-ethyl-N-nitrosourea)) or physical induction (e.g., irradiation with UV or gamma rays). In plant development, TILLING is well-known to introduce small modification like SNPs.

Site-specific modification may be achieved by introducing a site-specific nuclease or an active fragment thereof. Site-specific DNA cleaving activities of meganucleases, zinc finger nucleases (ZFNs), transcription activator-like effector nucleases (TALENs), or the clustered regularly interspaced short palindromic repeat (CRISPR), mainly the CRISPR/Cas9 technology have been widely applied in site-directed modifications of animal and plant genomes. The nucleases cause double strand breaks (DSBs) at specific cleaving sites, which are repaired by nonhomologous end-joining (NHEJ) or homologous recombination (HR). More recently discovered CRISPR systems include CRISPR/Cpf1, CRISPR/C2c2, CRISPR/CasX, CRISPR/CasY and CRISPR/Cmr, CRISPR/MAD7 or CRISPR/CasZ. Recombinases and Transposases catalyze the exchange or relocation of specific target sequences and can therefore also be used to create targeted modifications.

Furthermore, a base editing technique can be used to introduce a point mutation. Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalysed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Gaudelli et al., 2017 (Programmable base editing of A·T to G·C in genomic DNA without DNA cleavage. Nature, 551(7681), 464.) described adenine base editors (ABEs) that mediate the conversion of A·T to G·C in genomic DNA.

Prime editor systems are disclosed in Anzalone et al., 2019 (Search-and-replace genome editing without double-strand breaks or donor DNA, Nature, 576, 149-157). Base editing as detailed above, does not cut the double-stranded DNA, but instead uses the CRISPR targeting machinery to shuttle an additional enzyme to a desired sequence, where it converts a single nucleotide into another. Many genetic traits in plants and certain susceptibility to diseases caused by plant pathogens are caused by a single nucleotide change, so base editing offers a powerful alternative for GE. But the method has intrinsic limitations and is said to introduce off-target mutations which are generally not desired for high precision GE. In contrast, Prime Editing (PE) systems steer around the shortcomings of earlier CRISPR based GE techniques by heavily modifying the Cas9 protein and the guide RNA. The altered Cas9 only “nicks” a single strand of the double helix, instead of cutting both. The new guide RNA, called a pegRNA (prime editing extended guide RNA), contains an RNA template for a new DNA sequence, to be added to the genome at the target location. That requires a second protein, attached to Cas9 or a different CRISPR effector nuclease: a reverse transcriptase enzyme, which can make a new DNA strand from the RNA template and insert it at the nicked site. To this end, an additional level of specificity is introduced into the GE system in view of the fact that a further step of target specific nucleic acid::nucleic acid hybridization is required. This may significantly reduce off-target effects. Further, the PE system may significantly increase the targeting range of a respective GE system in view of the fact that BEs cannot cover all intended nucleotide transitions/mutations (C→A, C→G, G→C, G→T, A→C, A→T, T→A, and T→G) due to the very nature of the respective systems, and the transitions as supported by BEs may require DSBs in many cell types and organisms.

In one embodiment of the method according to any of the embodiments described above, step (i) comprises introducing into the cell a site-specific nuclease or an active fragment thereof, or providing the sequence encoding the same, the site-specific nuclease inducing a single- or double-strand break at a predetermined location, preferably wherein the site-specific nuclease or the active fragment thereof comprises a zinc-finger nuclease, a transcription activator-like effector nuclease, a CRISPR/Cas system, including a CRISPR/Cas9 system, a CRISPR/Cpf1 system, a CRISPR/C2C2 system a CRISPR/CasX system, a CRISPR/CasY system, a CRISPR/Cmr system, a CRISPR/MAD7 system, a CRISPR/CasZ system, an engineered homing endonuclease, a recombinase, a transposase and a meganuclease, and/or any combination, variant, or catalytically active fragment thereof; and optionally when the site-specific nuclease or the active fragment thereof is a CRISPR nuclease: providing at least one guide RNA or at least one guide RNA system, or a nucleic acid encoding the same; and optionally providing at least one repair template nucleic acid sequence.

The introduction of the respective tool(s) in step i) may e.g., be achieved by means of transformation, transfection or transduction. Besides transformation methods based on biological approaches, like Agrobacterium transformation or viral vector mediated plant transformation, methods based on physical delivery methods, like particle bombardment or microinjection, have evolved as prominent techniques for importing genetic material into a plant cell or tissue of interest. Helenius et al., 2000 (Gene delivery into intact plants using the Helios™ Gene Gun, Plant Molecular Biology Reporter, 18 (3):287-288) discloses a particle bombardment as physical method for transferring material into a plant cell. Currently, there are a variety of plant transformation methods to introduce genetic material in the form of a genetic construct into a plant cell of interest, comprising biological and physical means known to the skilled person on the field of plant biotechnology and which can be applied. Notably, said delivery methods for transformation and transfection can be applied to introduce the required tools simultaneously. A common biological means is transformation with Agrobacterium spp. which has been used for decades for a variety of different plant materials. Viral vector mediated plant transformation represents a further strategy for introducing genetic material into a cell of interest. Physical means finding application in plant biology are particle bombardment, also named biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alia transfection with calcium phosphate, transfection using liposomes, e.g., cationic liposomes, or transfection with cationic polymers, including DEAD-dextran or polyethylenimine, or combinations thereof. Every delivery method has to be specifically fine-tuned and optimized so that a construct of interest can be introduced into a specific compartment of a target cell of interest in a fully functional and active way. The above delivery techniques, alone or in combination, can be used to introduce the necessary constructs, expression cassettes or vectors carrying the required tools i.e. a site-specific effector complex or at least one subcomponent thereof, i.e., at least one site-specific nuclease, at least one guide RNA, at least one repair template, or at least one base editor, or the sequences encoding the aforementioned subcomponents, according to the present invention into a target cell, in vivo or in vitro.

After its import, e.g., by transformation or transfection by biological or physical means, the nucleic acid construct or the expression cassette can either persist extra-chromosomally, i.e., non-integrated into the genome of the target cell, for example in the form of a double-stranded or single-stranded DNA, a double-stranded or single-stranded RNA. Alternatively, the construct, or parts thereof, according to the present disclosure can be stably integrated into the genome of a target cell, including the nuclear genome or further genetic elements of a target cell, including the genome of plastids like mitochondria or chloroplasts. A nucleic acid construct or an expression cassette may also be integrated into a vector for delivery into the target cell or organism.

In the method of the present invention, the tools used for introducing the modifications or replacing the original promoter are preferably only transiently present/expressed in the cell and are not integrated into the genome.

In one embodiment of the method according to any of the embodiments described above, the expression level of the nucleic acid molecule of interest is increased synergistically with respect to a modification introduced only at the first or the second location.

Advantageously, the method of the present invention allows to synergistically increase the expression of a nucleic acid molecule of interest. The enhancement can be applied to the expression of a trait gene, i.e. a gene that provides desirable agronomic traits such as resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, resistance or tolerance to 2,4-D, protoporphyrinogen oxidase (PPO) inhibitors, ALS inhibitors, and Dicamba, a nucleic acid molecule encoding resistance or tolerance to biotic stress, including a viral resistance gene, a fungal resistance gene, a bacterial resistance gene, an insect resistance gene, or a nucleic acid molecule encoding a yield related trait, including lodging resistance, flowering time, shattering resistance, seed color, endosperm composition, or nutritional content.

The trait gene can be an endogenous gene to the plant cell, but it can also be a transgene, which was introduced into the plant cell by biotechnological means, optionally together with the promoter controlling its expression.

The present invention also relates to a plant cell, or a plant obtained or obtainable by a method according to any of the embodiments described above. Preferably, the plant cell or plant according to the invention is not a product of an essentially biological process.

In one embodiment, the plant cell is derived from, or the plant is a plant of a genus selected from the group consisting of Beta, Zea, Triticum, Secale, Sorghum, Hordeum, Saccharum, Oryza, Solanum, Brassica, Glycine, Gossipium and Helianthus.

In a preferred embodiment, the plant cell is derived from Zea mays (Zm) or Beta vulgaris (Bv).

Finally, the present invention also relates to the use of a nucleic acid molecule according to any of the embodiments described above for increasing the expression level of a nucleic acid molecule of interest in a plant cell, preferably in a method according to any of the embodiments described above. Preferably, the expression level of the nucleic acid molecule of interest is synergistically increased.

### EXAMPLES

In the examples, activation of one corn (Zm) and two sugar beet (Bv) promoters is demonstrated upon introduction of a combination of a cis-regulatory element (CRE) and a core promoter element (CPE).

The respective promoters were cloned and placed in front of a luciferase (NLuc) reporter gene. Modified versions of the promoters were created by using oligo ligation and site directed mutagenesis to introduce the CRE and CPE. Bombardment of corn or sugar beet leaf explants was followed by luciferase measurement to assess the impact of the modifications on promoter activity.

### Example 1: Combinations of CRE and CPE in the ZmCW13 Promoter

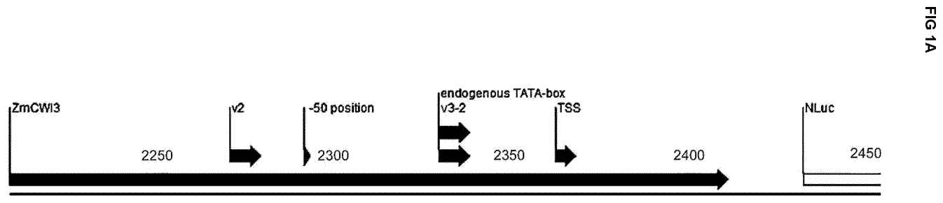

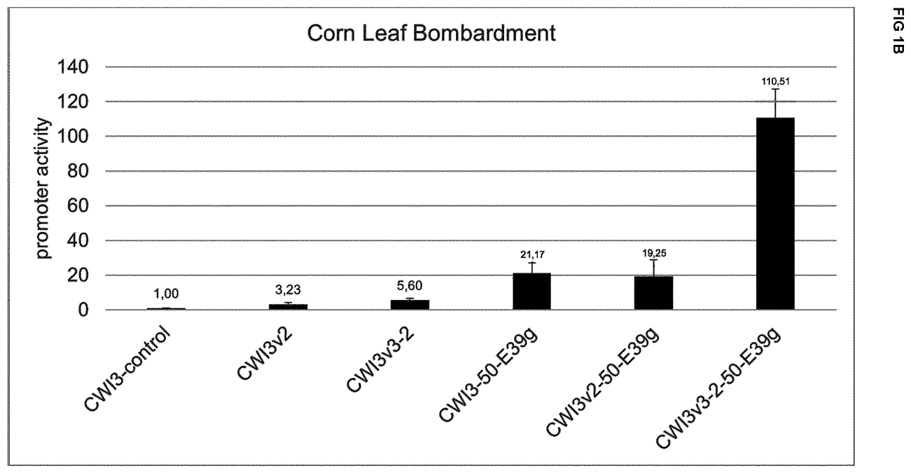

The sequence of ZmCWI3 is given in SEQ ID NO: 184. The insertion of a CRE (E039g, SEQ ID NO: 5) in combination with an optimized TATA box (CTATAAATA) in the ZmCWI3 promoter led to a 110-fold increase in expression (SEQ ID NO: 189), while the two modifications alone only achieved a 5,6- or 21,2-fold increase (SEQ ID NOs: 186 and 187). The CRE must be placed upstream of the TATA-box. If the CRE was placed downstream of the TATA-box, this resulted in a promoter activation not differing from the effect of the CRE alone (SEQ ID NO: 188) (see FIG. 1).

### Example 2: Combination of CRE and CPE in the BvHPPD1 Promoter

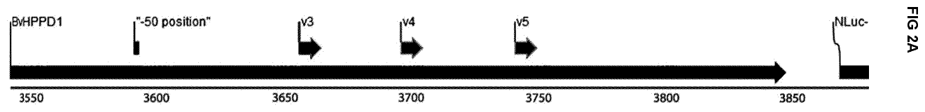

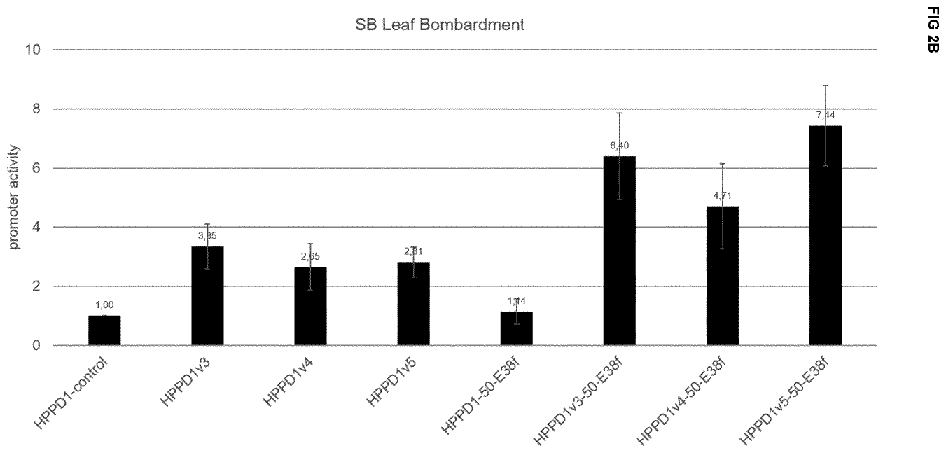

The sequence of the BvHPPD1 promoter is given in SEQ ID NO: 190. Addition of a CRE (E038f, SEQ ID NO: 6) alone had no significant effect in the sugar beet HPPD1 promoter (SEQ ID NO: 194). However, when it was combined with a TATA box (CTATAAATA), it had a significant effect and resulted in an approximate doubling of the promoter activation (SEQ ID NOs: 195, 196 and 197) compared to what was achieved by the TATA box insertion alone (SEQ ID NOs: 191, 192 and 193). This increased activation works within a window of ˜100 bp with respect to the TATA box, keeping a high level of flexibility concerning the positioning of modifications (see FIG. 2).

### Example 3: Combination of CRE and CPE in the Bv-prom3 Promoter

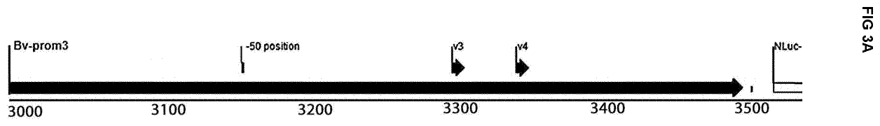

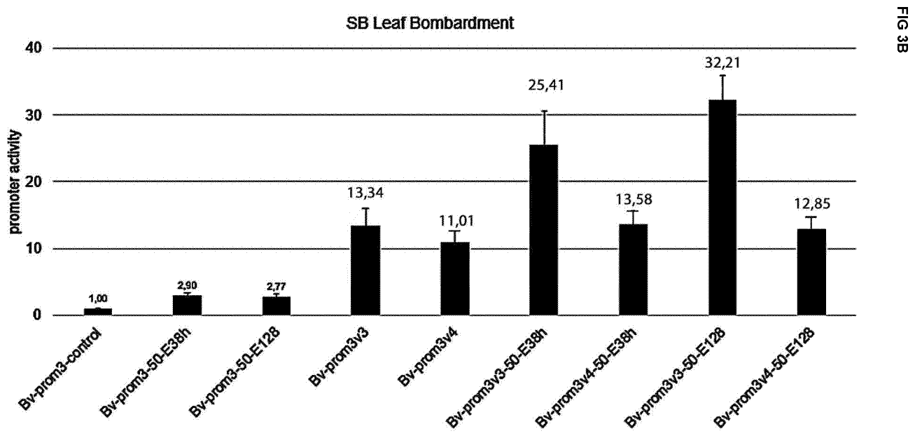

The Bv-prom3 promoter has a rather broad TSS around 290 bp upstream of the start codon and a weak endogenous TATA box at −320 bp upstream of the start codon. The Bv-prom3 promoter responded better to activation by TATA box insertion (11 to 13-fold) than to activation by CRE insertion (2,8 to 2,9-fold). TATA box insertion was performed by adding an additional TATA-box (CTATAAATA) at a position −197 bp upstream of the start codon by exchange of 4 bases and at a position −153 bp upstream of the start codon by exchange of 5 bases. Two different CREs (E038h, SEQ ID NO: 7 and E128, SEQ ID NO: 8) were inserted via element ligation at the −50 position relative to TSS which is positioned at −362 upstream of the start codon. The new approach using specific CPE-CRE combinations resulted in a much stronger activation (25 to 32-fold). These results also show that this approach is not restricted to one type of CRE as two very different types have been used here, an as1-like (E038h, SEQ ID NO: 7) and a double G-box (E128, SEQ ID NO: 8) element. In terms of the position the result for the Bv-prom3 promoter indicates that the distance between CRE and TATA box in this case should not exceed ˜160 bp (see FIG. 3).

### Example 4: Combination of Different CRE and CPE (TATA-Box) in the BvHPPD1 Promoter

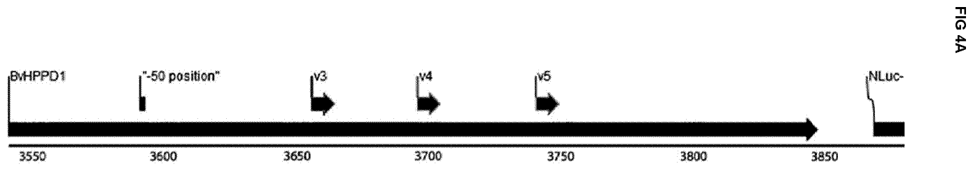

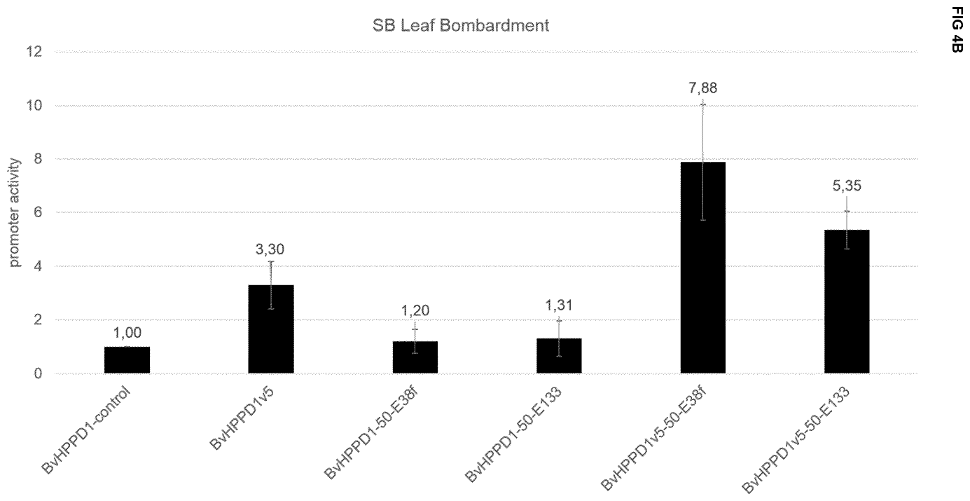

The sequence of the BvHPPD1 promoter is given in SEQ ID NO: 190. Addition of a CRE (E038f, SEQ ID NO: 6 or E133, SEQ ID NO: 199) alone had no significant effect in the sugar beet HPPD1 promoter (SEQ ID NO: 194 and SEQ ID NO: 205). However, when the CREs are combined with a TATA box (CTATAAATA), it had a significant effect and resulted in an approximate doubling of the promoter activation (SEQ ID NOs: 197 and 206) compared to what was achieved by the TATA box insertion alone (SEQ ID NOs: 193). This example shows that there is flexibility in the type of CRE used for synergistic activation. Besides an as1-like element E038f, another variant of a double G-box element E133 is functional in such approaches as well (see FIG. 4).

### Example 5: Combination of Different CRE and CPE (TATA-Box) in the BvHPPD2 Promoter

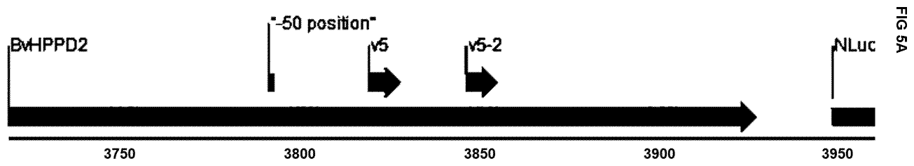

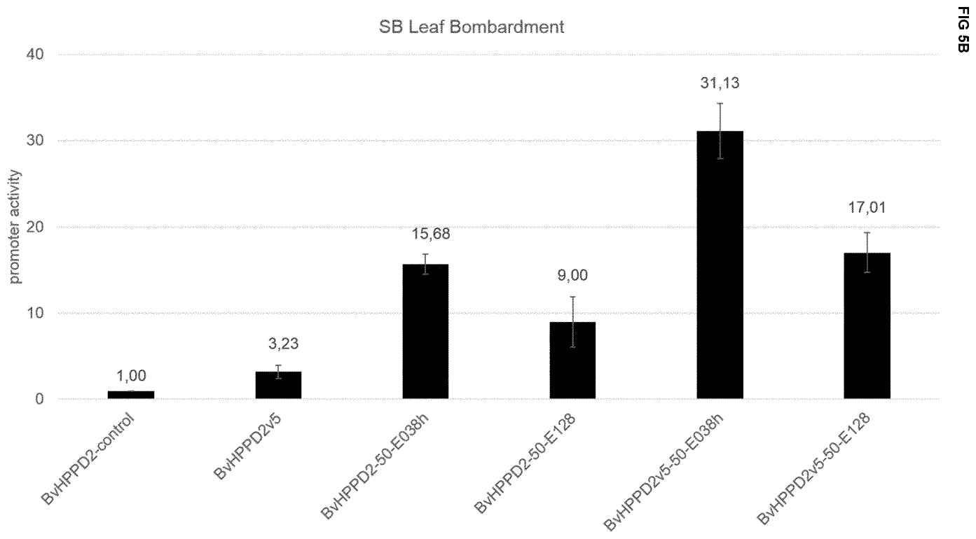

The sequence of the BvHPPD2 promoter is given in SEQ ID NO: 207. The BvHPPD2 responds better to activation by CRE insertion (9- to 16-fold) than to activation by TATA-box insertion at position v5 (3,2-fold). TATA box insertion was performed by adding an additional TATA-box (CTATAAATA) at a position −106 bp upstream of the start codon by exchange of 5 bases (SEQ ID NO: 208). Two different CREs (E038h, SEQ ID NO: 7 and E128, SEQ ID NO: 8) were inserted via element ligation at the −50 position relative to TSS which is positioned at −133 bp upstream of the start codon (SEQ ID NOs: 209 and SEQ ID NO: 210). The new approach using specific CPE-CRE combinations (SEQ ID NOs: 211 and 212) resulted in a synergistic activation (17 to 31-fold) of the BvHPPD2 promoter. In this case the distance between CRE and CPE is 27 bp. This example also shows that this approach is not restricted to one type of CRE as two very different types have been used here, an as1-like (E038h, SEQ ID NO: 7) and a double G-box (E128, SEQ ID NO: 8) element (see FIG. 5).

### Example 6: Combination of Different CRE and CPE (TATA-Box) in the Zm-prom6 Promoter

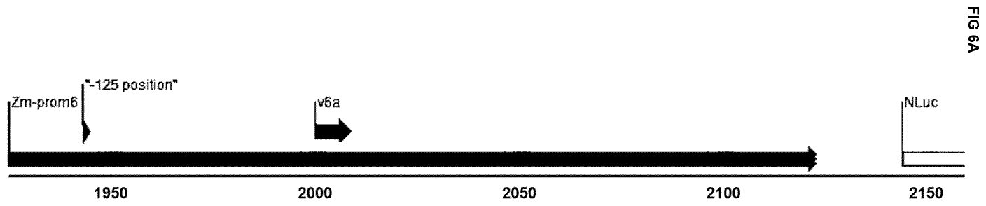

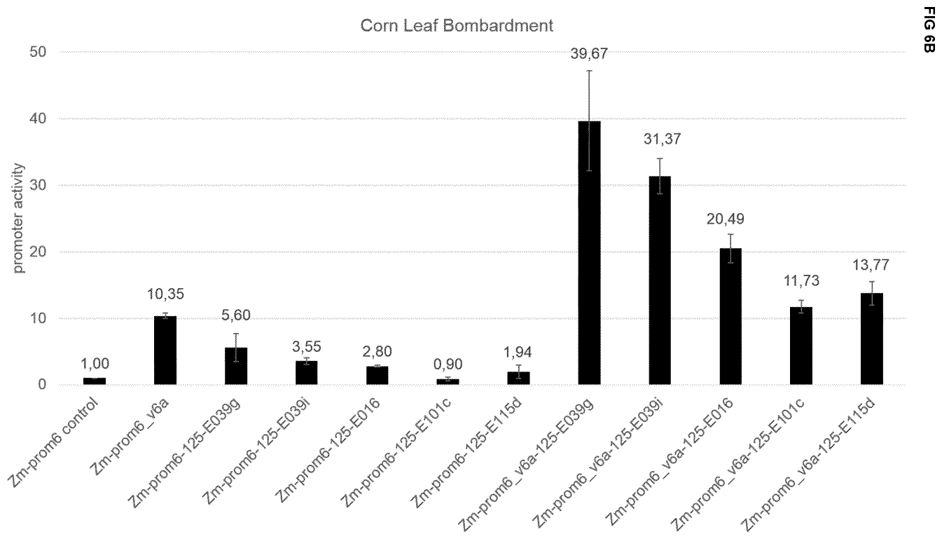

The Zm-prom6 promoter has got a TSS around 50 bp upstream of the start codon and an endogenous TATA box 83 bp upstream of the start codon. The Zm-prom6 promoter moderately responds to activation by TATA-box insertion (3 to 10-fold) and to activation by CRE insertion (up to 5,6-fold). An additional TATA-box (CTATAAATA) is generated at a position v6a, −121 bp upstream of the start codon by exchange of 7 bases. Different CREs like the as1-like elements E039g (SEQ ID NO: 5) and E039i (SEQ ID NO: 198), the TEF-box promoter motif E016 (SEQ ID NO: 200), a corn CYP promoter fragment E101c (SEQ ID NO: 201) and the corn adh1 promoter element E115d (SEQ ID NO: 202) are inserted via element ligation at the −125 position relative to the TSS which is positioned at −177 bp upstream of the start codon. The new approach using specific CPE-CRE combinations resulted in a much stronger activation (12 to 40-fold) compared to TATA-box or CRE insertion alone. This example again shows that this approach is not restricted to one type of CRE (see FIG. 6).

### Example 7: Combination of CRE and CPE (Y-Patch) in the BvFT2 Promoter

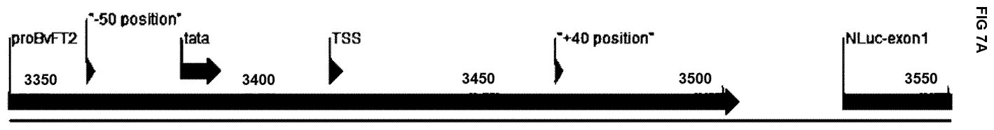

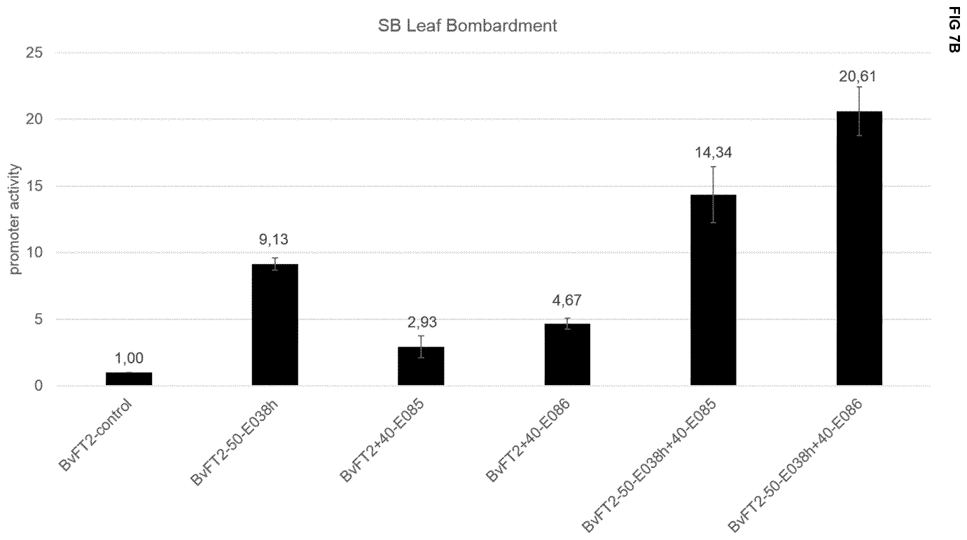

The activity of the BvFT2 promoter can be increased 9-fold by insertion of the CRE E038h (SEQ ID NO: 7) in the −50 position (SEQ ID NO: 214). Insertion of a Y-patch E085 (SEQ ID NO: 203) or E086 (SEQ ID NO: 204) in position +40 (SEQ ID NOs: 215 and 216) leads to an increase of 2,9-fold or 4,7-fold, respectively. The magnitude of effect correlates with a longer Y-patch sequence. The combined addition of a CRE and a Y-patch CPE to the BvFT2 promoter results in synergistic promoter activation of 14-fold and 21-fold (SEQ ID NO: 217 and SEQ ID NO: 218). In this case the distance between CRE and CPE is 104 bp. The longer Y-patch element E086 leads to a more pronounced effect of 21-fold increase in promoter activity (see FIG. 7). This example shows that synergistic activation effects cannot only be achieved by combining CRE with TATA-box CPE but also by combining with Y-patch CPE. The positioning would also allow for the combination of CRE+TATA-box CPE+Y-patch CRE, giving even more flexibility and options to apply synergistic promoter activation.

### Example 8: Combination of CRE and CPE in the Zm-prom2 Promoter (Distance Between CRE and CPE)

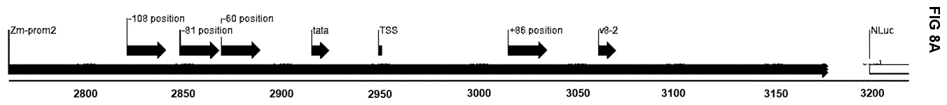

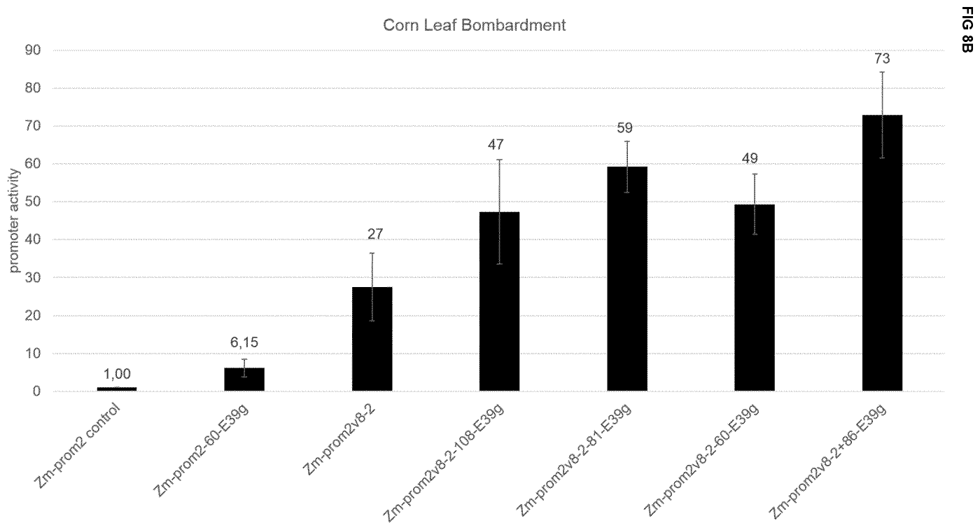

The Zm-prom2 promoter has got a TSS around 225 bp upstream of the start codon and an endogenous TATA-box 261 bp upstream of the start codon. The Zm-prom2 promoter moderately responds to activation by CRE insertion (6-fold, exemplary) and well to TATA box insertion (27-fold). The additional TATA-box (CTATAAATA) is generated at a position v8-2, 115 bp upstream of the start codon by exchange of 3 bases. The as1-like element E039g (SEQ ID NO: 5) is inserted via site-directed mutagenesis at different positions upstream of the generated TATA-box in position v8-2. The promoter modifications are covering the following distances between CRE and CPE: 27 bp distance with CRE in position +86 (161 bp upstream of the start codon), 172 bp distance with CRE in position −60, 193 bp distance with CRE in position −81 and 220 bp distance with CRE in position −108. From 27 bp to 220 bp distance between CRE and CPE synergistic enhancement of expression is observed, emphasizing the flexibility of our new approach with respect to the distance between CRE and CPE (see FIG. 8).

### Example 9: Combination of CRE and CPE in the ZmCW13 Promoter (Distance Between CRE and CPE)

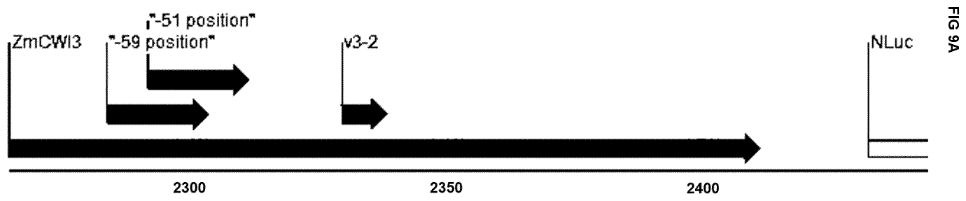

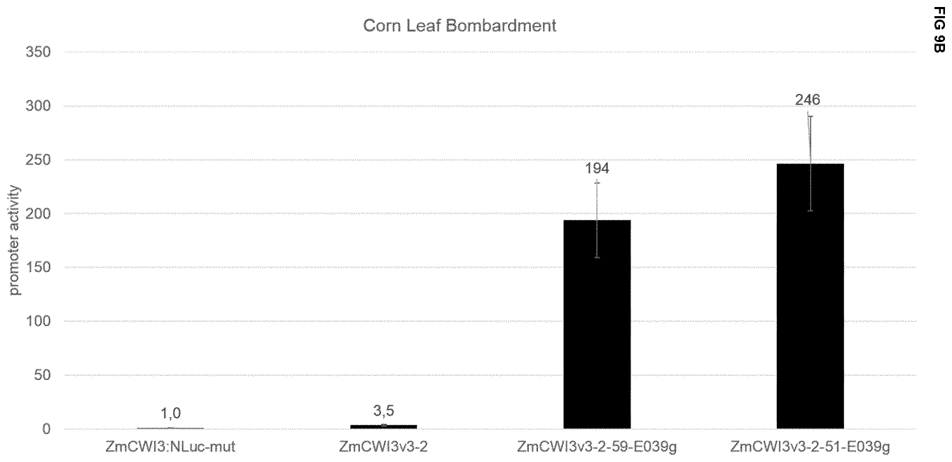

The sequence of ZmCWI3 is given in SEQ ID NO: 184. In CWI3v3-2, the endogenous TATA box (CTACAAATA) was optimized by one point mutation to CTATAAATA (SEQ ID NO: 186). In CWI3v3-2-59-E039g, an as1-like CRE (E039g, SEQ ID NO: 5) is generated via site-directed mutagenesis at the −59 position, which is at a 26 bp distance to position v3-2 (SEQ ID NO: 220). In CWI3v3-2-51-E039g, an as1-like CRE (E039g, SEQ ID NO: 5) is generated via site-directed mutagenesis at the −51 position, which is at an 18 bp distance to position v3-2 (SEQ ID NO: 219). The new approach of combining CRE and CPE leads to synergistic promoter activation of 194-fold and 246-fold. The 18 bp distance between CRE and CPE works optimal to achieve maximal effects with our synergistic promoter activation approach (see FIG. 9).

### Example 10: Combination of CRE and CPE in the Zm-prom7 Promoter (Distance Between CRE and CPE)

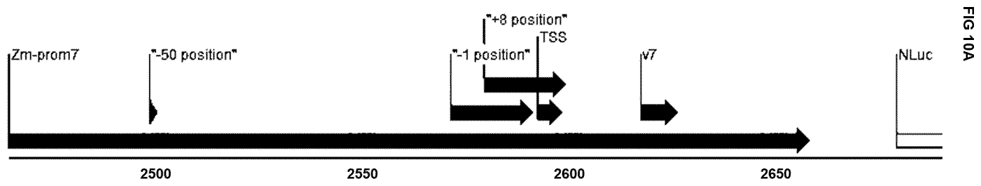

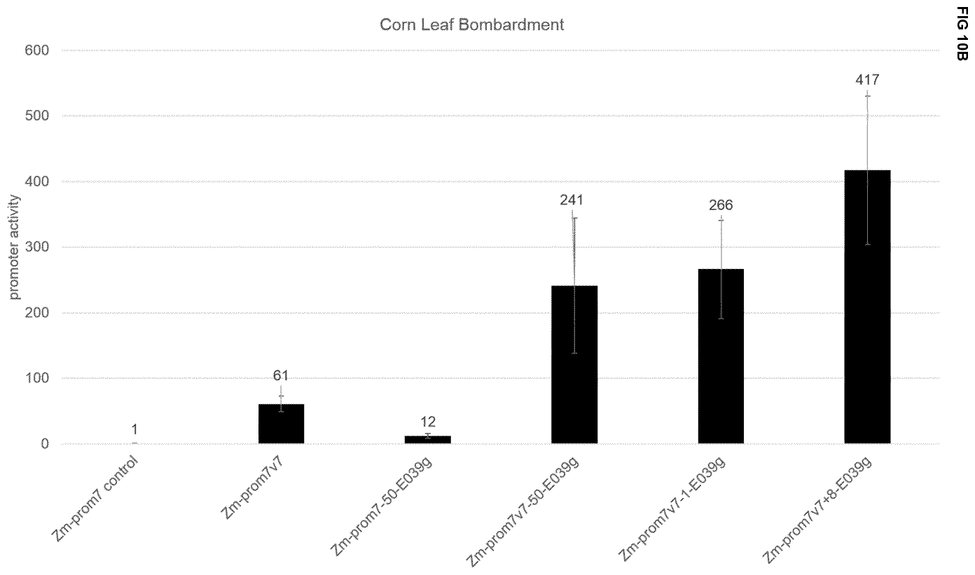

The Zm-prom7 promoter strongly responds to TATA box insertion in position v7 (61-fold) and to activation by CRE insertion in position −50 (12-fold). The additional TATA-box (CTATAAATA) is generated at a position v7, 39 bp upstream of the start codon by exchange of 7 bases. The as1-like element E039g (SEQ ID NO: 5) is inserted via site-directed mutagenesis or oligo ligation at different positions upstream of the generated TATA-box in position v7 (Zm-prom7v7-50-E039g, Zm-prom7v7-1-E039g and Zm-prom7v7+8-E039g). These promoter modifications are covering the following distances between CRE and CPE: 118 bp distance with CRE in position −50 (157 bp upstream of the start codon), 26 bp distance with CRE in position −1 and 18 bp distance with CRE in position +8. The new approach of combining CRE and CPE leads to synergistic promoter activation in the range of 241-fold to 417-fold. The 18 bp distance between CRE and CPE works optimal to achieve maximal effects with our synergistic promoter activation approach (see FIG. 10).

### Example 11: Combination of CRE and CPE in the Zm-prom8 Promoter (Strategy for Maximal Effects)

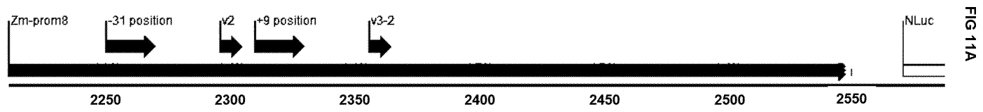

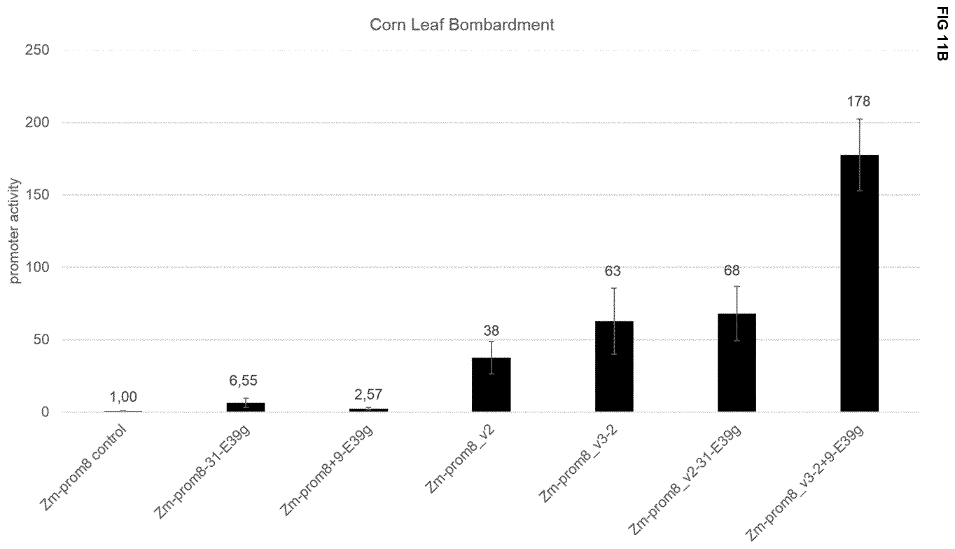

The Zm-prom8 promoter strongly responds to TATA box insertion in position v2 (38-fold) and even stronger to TATA box insertion in position v3-2 (63-fold). The two positions are located 252 bp (v2) or 192 bp (v3-2) upstream of the start codon. The additional TATA-box (CTATAAATA) is generated at position v2 by exchange of 5 bases and at position v3-2 by exchange of 6 bases. Insertion of the as1-like element E039g (SEQ ID NO: 5) in position −31 of the Zm-prom8 via site-directed mutagenesis results in 6,6-fold activation while the insertion in position +9 leads to 2,6-fold activation. The position −31 is located 298 bp upstream of the start codon, the position +9 is located 238 bp upstream of the start codon. The new approach of combining CRE and CPE by generating the promoter variants Zmprom8_v2-31-E39g or Zm-prom8_v3-2+9-E39g leads to synergistic promoter activation of 68-fold and 178-fold, respectively. The distance between CRE and CPE is 26 bp in both cases indicated that the optimal position for the generated TATA-box is more important than the position of the CRE if the aim is the achievement of maximal promoter activating effects (see FIG. 11).

This finding leads to a step-wise approach in identifying the promoter modification with the largest activating effect. Step1: Find the optimal position to generate an activating CPE. Step2: Place the CRE in optimal distance upstream of the CPE.

1. A method for increasing the expression level of a nucleic acid molecule of interest in a plant cell, the method comprising
(i) introducing a modification in the nucleic acid sequence of an endogenous promoter controlling the expression of the nucleic acid molecule of interest in a first location so that a cis-regulatory element selected from an as1-like element, a G-box element, a double G-box element, a TEF-box promoter motif, a corn CYP promoter fragment and a corn adh1 promoter element is formed and in a second location so that a core promoter element selected from a TATA box motif, a Y-patch motif, an initiator element and a downstream promoter element is formed, and
(ii) obtaining at least one plant cell showing an increased expression level of the nucleic acid molecule of interest compared to the expression level of the nucleic acid molecule of interest under the control of the unmodified endogenous promoter,
(iii) optionally, culturing the at least one plant cell obtained in step (ii) to obtain a plant showing an increased expression level of the nucleic acid molecule of interest compared to the expression level of the nucleic acid molecule of interest under the control of the unmodified endogenous promoter,
wherein the first location is located upstream of the second location and the first and the second location are located at a distance of 5 to 225 nucleotides from each other, preferably at a distance of 10 to 160 nucleotides.

2. The method of claim 1, wherein a second location is identified at a position −300 to −60 nucleotides relative to the start codon of the nucleic acid molecule of interest.

3. The method of claim 1, wherein at least one of the first and the second location is located downstream of the transcription start site.

4. The method of claim 1, wherein in step (i) less than 30 nucleotides are inserted, deleted and/or substituted at the first and/or the second location, preferably less than 25 nucleotides, preferably less than 20 nucleotides, preferably less than 15 nucleotides.

5. The method according to claim 1, wherein the modification in the first and/or second location is introduced by mutagenesis or by site-specific modification techniques using a site-specific nuclease or an active fragment thereof and/or a base editor and/or a prime editor.

6. The method of claim 1, wherein step (i) comprises introducing into the cell a site-specific nuclease or an active fragment thereof, or providing the sequence encoding the same, the site-specific nuclease inducing a single- or double-strand break at a predetermined location, preferably wherein the site-specific nuclease or the active fragment thereof comprises a zinc-finger nuclease, a transcription activator-like effector nuclease, a CRISPR/Cas system, including a CRISPR/Cas9 system, a CRISPR/Cpf1 system, a CRISPR/C2C2 system a CRISPR/CasX system, a CRISPR/CasY system, a CRISPR/Cmr system, a CRISPR/MAD7 system, a CRISPR/CasZ system, an engineered homing endonuclease, a recombinase, a transposase and a meganuclease, and/or any combination, variant, or catalytically active fragment thereof; and optionally when the site-specific nuclease or the active fragment thereof is a CRISPR nuclease: providing at least one guide RNA or at least one guide RNA system, or a nucleic acid encoding the same; and optionally providing at least one repair template nucleic acid sequence.

7. The method according to claim 1, wherein the core promoter element is a TATA box motif having the sequence of CTATAAATA.

8. The method according to claim 1, wherein the cis-regulatory element is selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or has a sequence being 95%, 96%, 97%, 98% or 99% identical to any of the sequences of SEQ ID NOs: 5 to 8 or 198 to 202.

9. The method according to claim 1, wherein the first and the second location are located at a distance of 15 to 60 nucleotides from each other.

10. The method of claim 9, wherein the expression level of the nucleic acid of interest controlled by the modified endogenous promoter is increased at least 20-fold, increased at least 50-fold, increased at least 100-fold, increased at least 150-fold, increased at least 200-fold, increased at least 250-fold, increased at least 300-fold, increased at least 350-fold, increased at least 400-fold in comparison to the expression level of the nucleic acid molecule of interest under the control of the unmodified endogenous promoter.

11. A promoter, which is endogenous to a plant cell and which has been modified to provide an increased expression level of a nucleic acid molecule of interest in a plant cell, wherein the promoter has been modified to comprise,
(a) a cis-regulatory element, which is heterologous to the promoter, selected from an as1-like element, a G-box element, a double G-box element, a TEF-box promoter motif, a corn CYP promoter fragment and a corn adh1 promoter element, and
(b) a TATA box motif having the sequence of CTATAAATA and being heterologous to the promoter,
wherein the cis-regulatory element is located upstream of the TATA box motif and the cis-regulatory element and the TATA box motif are positioned at a distance of 5 to 225 nucleotides from each other, preferably positioned at a distance of 10 to 160 nucleotides from each other, and wherein the expression level provided by the endogenous modified promoter is increased synergistically with respect to the endogenous promoter comprising only said cis-regulatory element or said TATA box motif sequence.

12. The modified promoter of claim 11, wherein at least one of the cis-regulatory element and the TATA box motif are located downstream of the transcription start site.

13. The modified promoter according to claim 11, wherein the modified promoter provides an increased expression level of a nucleic acid molecule of interest compared to the expression level of a nucleic acid molecule of interest under the control of the unmodified endogenous promoter.

14. The modified promoter according to claim 11, wherein the cis-regulatory element and the TATA box motif are located at a distance of 15 to 60 nucleotides from each other.

15. The modified promoter of claim 14, wherein the expression level of a nucleic acid of interest controlled by the modified endogenous promoter is increased at least 20-fold, increased at least 50-fold, increased at least 100-fold, increased at least 150-fold, increased at least 200-fold, increased at least 250-fold, increased at least 300-fold, increased at least 350-fold, increased at least 400-fold in comparison to the expression level of the nucleic acid molecule of interest under the control of the unmodified endogenous promoter.

16. The modified promoter according to claim 11, wherein the cis-regulatory element is selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or has a sequence being 95%, 96%, 97%, 98% or 99% identical to any of the sequences of SEQ ID NOs: 5 to 8 or 198 to 202.

17. A nucleic acid molecule comprising a promoter sequence, which is endogenous to a plant cell and which has been modified to comprise;
(a) a cis-regulatory element selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or having a sequence being 95%, 96%, 97%, 98% or 99% identical to any of the sequences of SEQ ID NOs: 5 to 8 or 198 to 202, and
(b) a TATA box motif having the sequence of CTATAAATA, located at a position −300 to −60 nucleotides relative to the start codon,
wherein (a) and (b) are located at a distance of 15 to 60 nucleotides to each other,
and wherein the expression level provided by the modified endogenous promoter is increased at least 20-fold with respect to a promoter comprising no modification and wherein the expression level provided by the promoter is increased synergistically with respect to an endogenous promoter comprising only said cis-regulatory element or said TATA box motif.

18. The nucleic acid molecule of claim 17, wherein at least one of the cis-regulatory element and the core promoter element are located downstream of the transcription start site.

19. A plant cell or a plant obtained or obtainable by a method according to claim 1.

20. A method of using a nucleic acid molecule for increasing the expression level of a nucleic acid molecule of interest in a plant cell,
wherein the nucleic acid molecule comprises a promoter sequence, which is endogenous to a plant cell and which has been modified to comprise:
(a) a cis-regulatory element selected from the group consisting of E039g (SEQ ID NO: 5), E038f (SEQ ID NO: 6), E038h (SEQ ID NO: 7), E128 (SEQ ID NO: 8), E133 (SEQ ID NO: 199), E039i (SEQ ID NO: 198), E016 (SEQ ID NO: 200), E101c (SEQ ID NO: 201) and E115d (SEQ ID NO: 202) or having a sequence being 95%, 96%, 97%, 98% or 99% identical to any of the sequences of SEQ ID NOs: 5 to 8 or 198 to 202, and
(b) a TATA box motif having the sequence of CTATAAATA, located at a position −300 to −60 nucleotides relative to the start codon,
wherein (a) and (b) are located at a distance of 15 to 60 nucleotides to each other,
preferably in a method according to claim 1.
